(19) United States
(12) Patent Application Publication
(10) Pub. No.: US 2023/0081756 A1
(43) Pub. Date: Mar. 16, 2023

(54) COMPOSITIONS COMPRISING BACTERIAL SPECIES AND METHODS RELATED THERETO

(71) Applicant: XBIOME INC., New York, NY (US)

(72) Inventors: Jackie PAPKOFF, South San Francisco, CA (US); Jun MA, South San Francisco, CA (US); Jason HUDAK, South San Francisco, CA (US); Linh NGUYEN, South San Francisco, CA (US)

(21) Appl. No.: 17/790,378

(22) PCT Filed: Dec. 31, 2020
(86) PCT No.: PCT/US20/67685

§ 371 Date: Jul. 1, 2022

Related U.S. Application Data
(60) Provisional application No. 62/955,951, filed on Dec. 31, 2019.
(60) Provisional application No. 62/959,558, filed on Jan. 10, 2020.
(60) Provisional application No. 62/975,827, filed on Feb. 13, 2020.
(60) Provisional application No. 63/035,128, filed on Jun. 5, 2020.

(51) Int. Cl. A61K 35/74; C12N 1/20; A61K 47/26; A61K 35/747; A61K 39/395; A61P 35/00; A23L 33/135; A23L 33/21

(52) U.S. Cl. CPC A61K 35/74; C12N 1/205; A61K 47/26; A61K 35/747; A61K 39/3955; A61P 35/00; A23L 33/135; A23L 33/21; C12R 2001/01

(57) ABSTRACT

The disclosure relates generally to bacterial strains of the genus Butyricimonas, e.g., Butyricimonas faecihominis bacterial strains, and compositions, e.g., pharmaceutical compositions, comprising such strains. The disclosure further relates to methods of using such strains and compositions for preventing or treating a disorder, e.g., a cancer, when administered to a subject in need thereof.

### CROSS REFERENCE TO RELATED APPLICATIONS

This application claims the benefit of, and priority to, U.S. provisional patent application Ser. No. 62/955,951, filed Dec. 31, 2019, U.S. provisional patent application Ser. No. 62/959,558, filed Jan. 10, 2020, U.S. provisional patent application Ser. No. 62/975,827, filed Feb. 13, 2020, and U.S. provisional patent application Ser. No. 63/035,128, filed Jun. 5, 2020, each of which are hereby incorporated by reference herein in their entirety.

### SEQUENCE LISTING

The instant application contains a Sequence Listing which has been filed electronically in ASCII format and is hereby incorporated by reference in its entirety. Said ASCII copy, created on Dec. 30, 2020, is named ASP-061WO_SL.txt and is 6,415,350 bytes in size.

### BACKGROUND

The gastrointestinal tract (GI), as well as other organ systems, is a complex biological system that includes a community of many different organisms, including diverse strains of bacteria. Hundreds of different species may form a commensal community in the gastrointestinal tract and other organs in a healthy person. Moreover, microorganisms present in the gut not only play a crucial role in digestive health, but also influence the immune system. A disturbance or imbalance in a biological system, e.g., the gastrointestinal tract, may include changes in the types and numbers of bacteria in the gut which may lead to the development of, or may be an indicator of, an unhealthy state and/or disease.

### SUMMARY

The disclosure relates generally to bacterial strains, bacterial strain mixtures, and compositions comprising bacterial strains of the genus Butyricimonas. In some embodiments, the disclosed bacterial strains, bacterial strain mixtures or compositions are useful for preventing and/or treating cancer.

In one aspect, provided herein is a composition, for example, a pharmaceutical composition, comprising a bacterial strain of the genus Butyricimonas. In some embodiments, the composition further comprises an excipient, diluent and/or carrier. In some embodiments, the bacterial strain in the composition is lyophilized, freeze dried or spray dried.

In some embodiments, the composition or the bacterial strain in the composition is capable of increasing production of at least one pro-inflammatory gene product, e.g., interferon gamma (IFN-γ), interleukin 1 beta (IL-113), interleukin 6 (IL-6), interleukin 12 (IL-12, e.g., IL-12p40, IL-12p70), interleukin 23 (IL-23), interleukin 27 (IL-27), tumor necrosis factor (TNF), and/or TNF-related apoptosis inducing ligand (TRAIL), in a human cell, e.g., a macrophage (e.g., a THP-1 macrophage), a monocyte, a peripheral blood mononuclear cell (PBMC), or a monocyte-derived dendritic cell. For example, in some embodiments, the composition increases production of at least one pro-inflammatory gene product, e.g., IFN-γ, IL-1β, IL-6, IL-12p40, IL-12p70, IL-23, IL-27, TNF, and/or TRAIL, in a human cell, e.g., a macrophage (e.g., a THP-1 macrophage), a monocyte, a PBMC, or a monocyte-derived dendritic cell, when the human cell is contacted with the composition. In some embodiments, the composition or the bacterial strain in the composition is capable of increasing infiltration of T cells into a tumor.

In some embodiments, the composition comprises a bacterial strain of Butyricimonas that comprises a 16s rRNA gene sequence with at least about 98% sequence identity to the polynucleotide sequence of SEQ ID NO: 846. In some embodiments, the Butyricimonas bacterial strain comprises a 16s rRNA gene sequence with at least about 98.5%, 99% or 99.5% sequence identity to the polynucleotide sequence of SEQ ID NO: 846. In some embodiments, the Butyricimonas bacterial strain comprises a 16s rRNA gene sequence of SEQ ID NO: 846. In some embodiments, the Butyricimonas bacterial strain shares at least 70% DNA-DNA hybridization with strain Butyricimonas faecihominis P40-F2a, having the deposit accession number DSM 33411. In some embodiments, the Butyricimonas bacterial strain comprises a nucleotide sequence having at least about 70% identity to any one of SEQ ID NOs: 1-845. In some embodiments, the Butyricimonas bacterial strain comprises a genome having at least 95% average nucleotide identity (ANI) with the genome of Butyricimonas faecihominis strain P40-F2a, having the deposit accession number DSM 33411. In some embodiments, the Butyricimonas bacterial strain comprises a genome having at least 96.5% average nucleotide identity (ANI) and at least 60% alignment fraction (AF) with the genome of Butyricimonas faecihominis strain P40-F2a, having the deposit accession number DSM 33411. In some embodiments, the Butyricimonas bacterial strain is Butyricimonas faecihominis P40-F2a, having the deposit accession number DSM 33411.

In some embodiments, the Butyricimonas bacterial strain of the composition is viable. In some embodiments, the bacterial strain is capable of at least partially colonizing an intestine of a human subject. In some embodiments, the composition is suitable for oral delivery to a subject. In some embodiments, the composition is formulated as an enteric formulation. In some embodiments, the enteric formulation is formulated as a capsule, tablet, caplet, pill, troche, lozenge, powder, or granule. In some embodiments, the composition is formulated as a suppository, suspension, emulsion, or gel. In some embodiments, the composition comprises at least 1×10³ CFU of the bacterial strain.

In some embodiments, the composition comprises a therapeutically effective amount of the bacterial strain sufficient to prevent or treat a disorder when administered to a subject in need thereof. In some embodiments, the disorder is a cancer. Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma). In certain embodiments, the cancer is colorectal cancer (CRC).

In some embodiments, the composition comprises an excipient selected from the group consisting of a filler, a binder, a disintegrant, and any combination(s) thereof. In some embodiments, the excipient is selected from the group consisting of cellulose, polyvinyl pyrrolidone, silicon dioxide, stearyl fumarate or a pharmaceutically acceptable salt thereof, and any combination(s) thereof. In some embodiments, the composition further comprises a cryoprotectant. In some embodiments, the cryoprotectant is selected from the group consisting of a fructoligosaccharide, trehalose and a combination thereof. In some embodiments, the fructoligosaccharide is Raftilose® (fructooligosaccharide derived from inulin). In some embodiments, the composition is suitable for bolus administration or bolus release. In some embodiments, the composition comprises the Butyricimonas bacterial strain and at least one more additional bacterial strain(s).

In another aspect, provided herein is a bacterial strain, e.g., an isolated bacterial strain, of the genus Butyricimonas, wherein the bacterial strain comprises a 16s rRNA gene sequence with at least about 98% sequence identity to the polynucleotide sequence of SEQ ID NO: 846. In some embodiments, the Butyricimonas bacterial strain is capable of increasing production of at least one pro-inflammatory gene product, e.g., IFN-γ, IL-1β, IL-6, IL-12p40, IL-12p70, IL-23, IL-27, TNF, and/or TRAIL, in a human cell, e.g., a macrophage (e.g., a THP-1 macrophage), a monocyte, a PBMC, or a monocyte-derived dendritic cell. For example, in some embodiments, the Butyricimonas bacterial strain increases production of at least one pro-inflammatory gene product, e.g., IFN-γ, IL-1β, IL-6, IL-12p40, IL-12p70, IL-23, IL-27, TNF, and/or TRAIL, in a human cell, e.g., a macrophage (e.g., a THP-1 macrophage), a monocyte, a PBMC, or a monocyte-derived dendritic cell, when the human cell is contacted with the Butyricimonas bacterial strain.

In some embodiments, the Butyricimonas bacterial strain comprises a 16s rRNA gene sequence with at least about 98.5%, 99%, or 99.5% sequence identity to the polynucleotide sequence of SEQ ID NO: 846. In some embodiments, the Butyricimonas bacterial strain comprises a 16s rRNA gene sequence of SEQ ID NO: 846. In some embodiments, the Butyricimonas bacterial strain shares at least 70% DNA-DNA hybridization with strain Butyricimonas faecihominis P40-F2a, having the deposit accession number DSM 33411. In some embodiments, the Butyricimonas bacterial strain comprises a nucleotide sequence having at least about 70% identity to any one of SEQ ID NOs: 1-845. In some embodiments, the Butyricimonas bacterial strain comprises a genome having at least 95% average nucleotide identity (ANI) with the genome of Butyricimonas faecihominis P40-F2a, having the deposit accession number DSM 33411. In some embodiments, the Butyricimonas bacterial strain comprises a genome having at least 96.5% average nucleotide identity (ANI) and at least 60% alignment fraction (AF) with the genome of Butyricimonas faecihominis P40-F2a, having the deposit accession number DSM 33411. In some embodiments, the Butyricimonas bacterial strain is Butyricimonas faecihominis P40-F2a, having the deposit accession number DSM 33411. In some embodiments, the bacterial strain is capable of at least partially colonizing an intestine of a human subject.

In another aspect, provided herein is a food product comprising a Butyricimonas bacterial strain described herein, or a composition comprising a Butyricimonas bacterial strain described herein.

In another aspect, provided herein is a method of preventing or treating a cancer in a subject in need thereof. A contemplated method comprises administering to the subject an effective amount of a disclosed pharmaceutical composition, pharmaceutical unit, bacterial strain or bacterial strain mixture.

Also provided herein is a method of treating a dysbiosis in a subject in need thereof, the method comprising administering a Butyricimonas bacterial strain described herein or a composition comprising a Butyricimonas bacterial strain described herein (e.g., a therapeutically effective amount of a Butyricimonas bacterial strain described herein or a composition comprising a Butyricimonas bacterial strain described herein) to the subject. Also provided herein is a method of modifying a gut microbiome in a subject, the method comprising administering a Butyricimonas bacterial strain described herein or a composition comprising a Butyricimonas bacterial strain described herein (e.g., a therapeutically effective amount of a Butyricimonas bacterial strain described herein or a composition comprising a Butyricimonas bacterial strain described herein) to the subject. In some embodiments of the methods provided herein, the method further comprises administering a prebiotic to the subject. In some embodiments, the subject is selected from the group consisting of a human, a companion animal, or a livestock animal.

### DESCRIPTION OF THE DRAWINGS

The disclosure can be more completely understood with reference to the following drawings.

FIG. 1 depicts the effects of Butyricimonas faecihominis P40-F2a on (A) IL-27; (B) TRAIL; (C) IL-6; and (D) IFN-β production in human monocyte-derived dendritic cells (moDCs). moDCs were co-incubated with PBS only, the Butyricimonas faecihominis P40-F2a strain (2 doses), or an anti-inflammatory strain control (2 doses), and supernatants were collected and assayed for production of the indicated cytokine. Each test article was evaluated in 4 replicates and results are representative of at least two independent experiments. p value ≤0.05 (one-way ANOVA).

FIG. 2 depicts the effects of Butyricimonas faecihominis P40-F2a on (A) IL-12p′70; (B) IL-1β; and (C) IL-23 production in human moDCs. MoDCs were co-incubated with PBS only, the Butyricimonas faecihominis P40-F2a strain (2 doses), or an anti-inflammatory strain control (2 doses), and supernatants were collected and assayed for production of the indicated cytokine. Each test article was evaluated in 4 replicates and results are representative of at least two independent experiments. p value ≤0.05 (one-way ANOVA).

FIG. 3 depicts the effects of Butyricimonas faecihominis P40-F2a on (A) IFN-γ; (B) IL-1β; (C) IL-6; (D) TRAIL; and (E) TNF production in human peripheral blood mononuclear cells (PBMCs). PBMCs were co-incubated with PBS only, the Butyricimonas faecihominis P40-F2a strain (2 doses), or an anti-inflammatory strain control (2 doses), and supernatants were collected and assayed for production of the indicated cytokine. Each test article was evaluated in 4 replicates and results are representative of at least two independent experiments. p value ≤0.05 (one-way ANOVA).

FIG. 4 depicts the effects of Butyricimonas faecihominis P40-F2a on (A) IL-1β; (B) IL-12p40; (C) TNF; (D) CCL-18; and (E) IL-10 production in human THP-1 M2 macrophages. THP-1 M2 macrophages were co-incubated with PBS only, the Butyricimonas faecihominis P40-F2a strain (3 doses), or an anti-inflammatory strain control (3 doses), and supernatants were collected and assayed for production of the indicated cytokine. Each test article was evaluated in 4 replicates and results are representative of at least two independent experiments. p value ≤0.05 (one-way ANOVA).

FIG. 5 depicts the effects of administration of (A) Butyricimonas faecihominis P40-F2a and (B) an anti-PD-L1 antibody, respectively, on tumor volume (fold-change) compared to vehicle in a murine B16F10 melanoma model. (A) Tumor volume ratio relative to day 12-post cell implantation. Mean+SEM; n=10 (Butyricimonas faecihominis), n=15 (Vehicle). (B) Tumor volume ratio relative to day 12-post cell implantation. Mean+SEM; n=10 (Anti-PD-L1), n=15 (Vehicle).

FIG. 6 depicts the effects of administration of (A) Butyricimonas faecihominis P40-F2a and (B) an anti-PD-L1 antibody, respectively, on tumor volume (mm³) compared to vehicle in a murine B16F10 melanoma tumor model. (C) Effect of administration of Butyricimonas faecihominis P40-F2a, vehicle, and an anti-PD-L1 antibody, respectively, on tumor weight (g) in a murine B16F10 melanoma model. (A) Absolute tumor volume (mm³). Mean+SEM; n=10 (Butyricimonas faecihominis), n=15 (Vehicle). (B) Absolute tumor volume (mm³). Mean+SEM; n=10 (Anti-PD-L1), n=15 (Vehicle). (C) Tumor weight on day 23 of study (mean+SEM); n=10 (Butyricimonas faecihominis, Anti-PD-L1), n=15 (Vehicle).

FIG. 7 depicts the effects of administration of (A) Butyricimonas faecihominis P40-F2a and (B) an anti-PD-L1 antibody, respectively, on body weight (g) compared to vehicle in a murine B16F10 melanoma model.

FIG. 8 depicts the effects of administration of (A) Butyricimonas faecihominis P40-F2a and (B) an anti-PD-1 antibody, respectively, on tumor volume (fold-change) compared to vehicle in a murine CT26 colon tumor model. (A) Tumor volume ratio relative to day 9-post cell implantation. Mean+SEM; n=10 (Butyricimonas faecihominis), n=15 (Vehicle). (B) Tumor volume ratio relative to day 9-post cell implantation. *P<0.05, 2-way ANOVA, Mean+SEM; n=10 (Anti-PD-1), n=15 (Vehicle).

FIG. 9 depicts the effects of administration of (A) Butyricimonas faecihominis P40-F2a and (B) an anti-PD-1 antibody, respectively, on tumor volume (mm³) compared to vehicle in a murine CT26 colon tumor model. (C) Effect of administration of Butyricimonas faecihominis P40-F2a, vehicle, and an anti-PD-1 antibody, respectively, on tumor weight (g) in a murine CT26 colon tumor model. (A) Absolute tumor volume (mm³). Mean+SEM; n=10 (Butyricimonas faecihominis), n=15 (Vehicle). (B) Absolute tumor volume (mm³). Mean+SEM; n=10 (Anti-PD-1), n=15 (Vehicle) (C) Tumor weight on day 24 of study (mean+SEM); n=10 (Butyricimonas faecihominis, Anti-PD-1), n=15 (Vehicle).

FIG. 10 depicts the effects of administration of (A) Butyricimonas faecihominis P40-F2a and (B) an anti-PD-1 antibody, respectively, on body weight (g) compared to vehicle in a murine CT26 colon tumor model.

FIG. 11 depicts the effects of administration of (A) Collinsella ASMB P121-D5a (C. P121-D5a); (B) Butyricimonas faecihominis P40-F2a; and (C) combination of Collinsella ASMB P121-D5a and Butyricimonas faecihominis P40-F2a strains, respectively, on tumor volume (left: mm³; right: fold-change) compared to vehicle in a murine CT26 colon tumor model. (A) Tumor volume and tumor volume change relative to 1^{st} day of dosing (day 10). Mean±SEM; n=15 (Vehicle), n=10 (Collinsella P121-D5a). (B) Tumor volume and tumor volume change relative to 1^{st} day of dosing (day 10). Mean±SEM; n=15 (Vehicle), n=10 (B. faecihominis). (C) Tumor volume and tumor volume change relative to 1^{st} day of dosing (day 10). Mean±SEM; n=15 (Vehicle), n=10 (Collinsella P121-D5a+B. faecihominis P40-F2a). ***P<0.001, significant difference compared to Vehicle on respective day by 2-way ANOVA with Dunnett post hoc test.

FIG. 12 depicts the effects of administration of (A) Collinsella ASMB P121-D5a; (B) Butyricimonas faecihominis P40-F2a; (C) an anti-PD-1 antibody; (D) combination of Collinsella ASMB P121-D5a and Butyricimonas faecihominis P40-F2a strains; and (E) combination of Collinsella ASMB P121-D5a and Butyricimonas faecihominis P40-F2a strains plus an anti-PD-1 antibody, respectively, on tumor volume (left: mm³; right: fold-change) compared to vehicle in a murine CT26 colon tumor model. Bacterial test articles were mixed with equal volumes of Vehicle prior to administration. (A) Collinsella ASMB P121-D5a induced a decrease in tumor volume (left), also depicted as tumor volume change (right). n=10 (C. P121-D5a+Vehicle), n=15 (Vehicle); mean±SEM. Significant difference compared to Vehicle on Day 19 **, Day 21 ****, Day 24 ****, Day 25 ****; **P<0.01, ****P<0.0001 by 2-way ANOVA with Dunnett post hoc test. (B) Butyricimonas faecihominis P40-F2a induced a decrease in tumor volume (left), also depicted as tumor volume change (right). n=10 (B. faecihominis+Vehicle), n=15 (Vehicle); mean±SEM. Significant difference compared to Vehicle on Day 24 **, Day 25 ***; **P<0.01, ***P<0.001 by 2-way ANOVA with Dunnett post hoc test. (C) α-PD-1 induced a decrease in tumor volume (left), also depicted as tumor volume change (right). n=10 (α-PD-1+Vehicle), n=15 (Vehicle); mean±SEM. Significant difference compared to Vehicle on Day 21 *, Day 24 **, Day 25 ***; **P<0.01, ***P<0.001 by 2-way ANOVA with Dunnett post hoc test. (D) The combination of Collinsella ASMB P121-D5a+Butyricimonas faecihominis P40-F2a induced a decrease in tumor volume (left), also depicted as tumor volume change (right). n=10 (Collinsella ASMB P121-D5a+Butyricimonas faecihominis P40-F2a), n=15 (Vehicle); mean±SEM. Significant difference compared to Vehicle on Day 21 *, Day 24 ***, Day 25 ****; *P<0.05, ***P<0.001, ****P<0.0001 by 2-way ANOVA with Dunnett post hoc test. (E) Collinsella ASMB P121-D5a+Butyricimonas faecihominis P40-F2a+α-PD-1 combination decreased tumor volume (left) and tumor volume change (right). n=10 (Collinsella ASMB P121-D5a+Butyricimonas faecihominis P40-F2a+α-PD-1), n=15 (Vehicle); mean±SEM. Significant difference compared to Vehicle on Day 19 **, Day 21 ****, Day 24 ****, Day 25 ****; **P<0.01, ****P<0.0001 by 2-way ANOVA with Dunnett post hoc test.

FIG. 13 depicts the effects of administration of (A) Alistipes senegalensis P150-D12a; (B) a Butyricimonas faecihominis strain; (C) an anti-PD-1 antibody; (D) the combination of Alistipes senegalensis P150-D12a and Butyricimonas faecihominis strains; and (E) the combination of Alistipes senegalensis P150-D12a and Butyricimonas faecihominis strains plus an anti-PD-1 antibody, respectively, on tumor volume (left: mm³; right: fold-change) compared to vehicle in a murine CT26 colon tumor model. Bacterial test articles were mixed with equal volumes of Vehicle prior to administration. (A) A. senegalensis induced a decrease in tumor volume (left), also depicted as tumor volume change (right). n=10 (A. senegalensis+Vehicle), n=15 (Vehicle); mean±SEM. Significant difference compared to Vehicle on Day 19 *, Day 21 **, Day 24 ****, Day 25 ****; *P<0.05, **P<0.01, ****P<0.0001 by 2-way ANOVA with Dunnett post hoc test. (B) B. faecihominis induced a decrease in tumor volume (left), also depicted as tumor volume change (right). n=10 (B. faecihominis+Vehicle), n=15 (Vehicle); mean±SEM. Significant difference compared to Vehicle on Day 24 **, Day 25 ***; **P<0.01, ***P<0.001 by 2-way ANOVA with Dunnett post hoc test. (C) α-PD-1 induced a decrease in tumor volume (left), also depicted as tumor volume change (right). n=10 (α-PD-1+Vehicle), n=15 (Vehicle); mean±SEM. Significant difference compared to Vehicle on Day 21 *, Day 24 **, Day 25 ***; *P<0.05, **P<0.01, ***P<0.001 by 2-way ANOVA with Dunnett post hoc test. (D) The combination of A. senegalensis+B. faecihominis induced a decrease tumor volume (left); also depicted as tumor volume change (right). n=10 (A. senegalensis+B. faecihominis), n=15 (Vehicle); mean±SEM. Significant difference compared to Vehicle on Day 21 **, Day 24 **, Day 25 ***; **P<0.01, ***P<0.001 by 2-way ANOVA with Dunnett post hoc test. (E) The combination of A. senegalensis+B. faecihominis+α-PD-1 induced a decrease in tumor volume (left), also depicted as tumor volume change (right). n=10 (A. senegalensis+B. faecihominis+α-PD-1), n=15 (Vehicle); mean±SEM. Significant difference compared to Vehicle on Day 19 **, Day 21 ****, Day 24 ****, Day 25 ****; **P<0.01, ****P<0.0001 by 2-way ANOVA with Dunnett post hoc test.

FIG. 14 depicts the effects of administration of a combination of Collinsella ASMB P121-D5a and Butyricimonas faecihominis P40-F2a strains plus an anti-PD-1 antibody on tumor-infiltrating CD8⁺ T cells in a murine CT26 colon tumor model. Shown are representative immunohistochemistry stainings for CD8 in formalin-fixed paraffin-embedded (FFPE) tumor sections following treatment with (A) Collinsella ASMB P121-D5a+Butyricimonas faecihominis P40-F2a (Ca+Bf); (B) Collinsella ASMB P121-D5a+Butyricimonas faecihominis P40-F2a+α-PD-1 (Ca+Bf+α-PD-1); (C) Vehicle; or (D) α-PD-1. (E) CD8-positive cells were quantified as a percentage of non-necrotic cells in whole tissue sections in blinded fashion. Ca, Collinsella ASMBP121-D5a; Bf, Butyricimonas faecihominis P40-F2a. *P<0.05, 1-way ANOVA with Dunnett's post hoc test; mean+SEM; n=10 per group, n=15 Vehicle.

FIG. 15 depicts the effects of administration of a combination of Alistipes senegalensis P150-D12a and Butyricimonas faecihominis P40-F2a strains plus an anti-PD-1 antibody on tumor-infiltrating CD8⁺ T cells in a murine CT26 colon tumor model. Shown are representative immunohistochemistry stainings for CD8 in FFPE tumor sections following treatment with (A) Alistipes senegalensis P150-D12a+Butyricimonas faecihominis P40-F2a (As+Bf); (B) Alistipes senegalensis P150-D12a+Butyricimonas faecihominis P40-F2a+an anti-PD-1 antibody (As+Bf+α-PD-1); (C) Vehicle; or (D) α-PD-1. (E) CD8-positive cells were quantified as a percentage of non-necrotic cells of whole tissue sections in blinded fashion. As, Alistipes senegalensis P150-D12a; Bf, Butyricimonas faecihominis P40-F2a. **P<0.01, 1-way ANOVA with Dunnett's post hoc test; mean+SEM; n=10 per group, n=15 Vehicle.

FIG. 16 depicts the effects of administration of a combination of Collinsella ASMB P121-D5a and Butyricimonas faecihominis P40-F2a strains with or without an anti-PD-1 antibody on tumor-infiltrating T cells in a murine CT26 colon tumor model, as determined by Nanostring RNA-based cell type profiling. RNA was extracted and purified from tumor samples and analyzed using the Mouse PanCancer TO 360 Panel. T cell designation was based on expression of Cd3d, Cd3e, Cd3g, Cd6, Sh2d1a and Trat1. Ca, Collinsella ASMB P121-D5a; Bf, Butyricimonas faecihominis P40-F2a. n=14 (Vehicle), n=9 (α-PD-1), n=10 (Ca+Bf) and n=9 (Ca+Bf+α-PD-1).

FIG. 17 depicts the effects of administration of a combination of Alistipes senegalensis P150-D12a and Butyricimonas faecihominis P40-F2a strains plus an anti-PD-1 antibody on tumor-infiltrating T cells in a murine CT26 colon tumor model, as determined by Nanostring RNA-based cell type profiling. RNA was extracted and purified from tumor samples and analyzed using the Mouse PanCancer TO 360 Panel. T cell designation was based on expression of Cd3d, Cd3e, Cd3g, Cd6, Sh2d1a and Trat1. As, Alistipes senegalensis P150-D12a; Bf, Butyricimonas faecihominis P40-F2a. n=14 (Vehicle), n=9 (α-PD-1), n=4 (As+Bf+α-PD-1).

FIG. 18 depicts the effects of administration of a combination of Alistipes senegalensis P150-D12a and Butyricimonas faecihominis P40-F2a strains plus an anti-PD-1 antibody on tumor-infiltrating CD8⁺ T cells in a murine CT26 colon tumor model, as determined by flow cytometry analysis of fresh tumor tissue. CD8⁺ T cells were identified from gating CD8⁺ CD4⁻ CD3⁺ CD45⁺ lymphocytes and quantified as percentage of live cells. As, Alistipes senegalensis P150-D12a; Bf, Butyricimonas faecihominis P40-F2a. ****P<0.0001, 1-way ANOVA with Dunnett's post hoc test; mean+SEM. n=8 (As+Bf+α-PD-1), n=15 (Vehicle), n=9 (α-PD-1).

FIG. 19 depicts the effects of administration of a combination of Collinsella ASMB P121-D5a and Butyricimonas faecihominis P40-F2a strains plus an anti-PD-1 antibody on IFN-γ in tumors in a murine CT26 colon tumor model. IFN-γ concentration was determined by MSD analysis of tumor tissue homogenate and normalized to homogenate protein concentration. Ca, Collinsella ASMB P121-D5a; Bf, Butyricimonas faecihominis P40-F2a. *P<0.05, **P<0.01, 1-way ANOVA with Dunnett's post hoc test; mean+SEM; n=10 (Ca+Bf), n=9 (Ca+Bf+α-PD-1), n=9 (α-PD-1) and n=15 (Vehicle).

FIG. 20 depicts the effects of administration of a combination of Alistipes senegalensis P150-D12a and Butyricimonas faecihominis P40-F2a strains plus an anti-PD-1 antibody on IFN-γ in tumors in a murine CT26 colon tumor model. IFN-γ concentration was determined by MSD analysis of tumor tissue homogenate and normalized to homogenate protein concentration. As, Alistipes senegalensis P150-D12a; Bf, Butyricimonas faecihominis P40-F2a. **P<0.01, 1-way ANOVA with Dunnett's post hoc test; mean+SEM; n=10 (As+Bf), n=5 (As+Bf+α-PD-1), n=15 (Vehicle) and n=9 (α-PD-1).

FIG. 21 depicts the effects of administration of Lactobacillus ruminis P167-B1a (Lr) and an anti-PD-L1 antibody, respectively, in a B16F10 tumor model. (A) Individual tumor volume (left panel) and mean tumor volume fold change (right panel). 2-way ANOVA with Dunnett's post hoc test compared to Vehicle; mean+SEM; ns=not significant. (B) Immunohistochemistry (IHC) analysis of CD3⁺ cells in fixed tumor sections, as a percentage of non-necrotic cells. Mean+SEM; ns=not significant.

FIG. 22 depicts the effects of administration of (A) Lactobacillus ruminis P167-B1a (Lr); (B) a combination of Lr and an anti-PD-1 antibody; (C) a combination of Butyricimonas faecihominis P40-F2a (Bf) and an anti-PD-1 antibody; and (D) a combination of Lr and Bf strains plus an anti-PD-1 antibody; each in comparison to administration of an anti-PD-1 antibody alone and vehicle alone, respectively, in a murine CT26 colon tumor model. (A) Lr induced a decrease in tumor volume (left), also depicted as tumor volume fold-change (right). n=10 (Lr; anti-PD-1), n=10 (Vehicle); mean±SEM. Significant difference compared to Vehicle on Day 25* by 2-way ANOVA with Dunnett post hoc test. Administration of Anti-PD-1 antibody also induced a decrease in tumor volume (significant difference compared to Vehicle on Day 21*, Day 24*** and Day 25**** by 2-way ANOVA with Dunnett post hoc test). (B) The combination of Lr and an anti-PD-1 antibody did not significantly increase tumor reduction in comparison to anti-PD-1 antibody alone. n=10 (Lr+anti-PD-1), n=10 (Vehicle); mean±SEM. Significant difference compared to Vehicle on Day 24** and Day 25*** by 2-way ANOVA with Dunnett post hoc test. (C) The combination of Bf and an anti-PD-1 antibody did not significantly increase tumor reduction in comparison to anti-PD-1 antibody alone. n=10 (Bf+anti-PD-1), n=10 (Vehicle); mean±SEM. Significant difference compared to Vehicle on Day 24*** and Day 25**** by 2-way ANOVA with Dunnett post hoc test. (D) The combination of Lr+Bf+anti-PD-1 antibody showed higher anti-tumor activity in comparison to anti-PD-1 antibody alone, and also compared to each of Lr+anti-PD-1 and Bf+anti-PD-1. n=10 (Lr+Bf+anti-PD-1), n=10 (Vehicle); mean±SEM. Significant difference compared to Vehicle on Day 21**, Day 24*** and Day 25*** by 2-way ANOVA with Dunnett post hoc test.

FIG. 23 depicts the effects of administration of a combination of Lactobacillus ruminis P167-B1a (Lr)+Butyricimonas faecihominis P40-F2a (Bf)+anti-PD-1 antibody on tumor infiltrating cells, compared to administration of anti-PD-1 antibody alone, as shown by immunohistochemistry (IHC) and flow cytometry (FC). (A, B) CD4⁺ T cells; (C, D) natural killer (NK) cells; (E) dendritic cells (DC); and (F) CD45⁺ leukocytes. *P<0.05, **P<0.01, ****P<0.0001 by 1-way ANOVA compared to Vehicle or α-PD-1 with Dunnett's post hoc test; mean+SEM.

FIG. 24 depicts the effects of administration of an anti-PD-1 antibody alone compared to: (A) a combination of Lactobacillus ruminis P167-B1a (Lr), Alistipes senegalensis P150-D12a (As) and Butyricimonas faecihominis P40-F2a (Bf) strains; (B) a combination of Lr+As+Bf; and a combination of Lr+As+Bf plus anti-PD-1 antibody, respectively; (C) a combination of Collinsella ASMB P121-D5a (Ca)+Lr+Bf strains; (D) a combination of Ca+Lr+Bf; and a combination of Ca+Lr+Bf plus anti-PD-1 antibody, respectively; and (E) Lr+Bf plus anti-PD-1 antibody, in a murine CT26 colon tumor model. (A) Lr+As+Bf induced a decrease in tumor volume (left), also depicted as tumor volume fold-change (right). n=10 (Lr+As+Bf; anti-PD-1), n=10 (Vehicle); mean±SEM. Significant difference compared to Vehicle on Day 21*, Day 24* and Day 25* by 2-way ANOVA with Dunnett post hoc test. Administration of Anti-PD-1 antibody also induced a decrease in tumor volume (significant difference compared to Vehicle on Day 24* and Day 25** by 2-way ANOVA with Dunnett post hoc test). (B) The combination of Lr+As+Bf+anti-PD-1 antibody significantly increased tumor reduction in comparison to anti-PD-1 antibody alone and Lr+As+Bf, respectively. n=10 (Lr+As+Bf; Lr+As+Bf+anti-PD-1; anti-PD-1), n=10 (Vehicle); mean±SEM. Significant difference compared to Vehicle, Lr+As+Bf, and anti-PD-1 alone, respectively, on Day 24** and Day 25** by 2-way ANOVA with Dunnett post hoc test. (C) Ca+Lr+Bf induced a decrease in tumor volume (left), also depicted as tumor volume fold-change (right). n=10 (Ca+Lr+Bf; anti-PD-1), n=10 (Vehicle); mean±SEM. Significant difference compared to Vehicle on Day 21*, Day 24** and Day 25*** by 2-way ANOVA with Dunnett post hoc test. (D) The combination of Ca+Lr+Bf+anti-PD-1 antibody significantly increased tumor reduction in comparison to anti-PD-1 antibody alone and Ca+Lr+Bf, respectively. n=10 (Ca+Lr+Bf; Ca+Lr+Bf+anti-PD-1; anti-PD-1), n=10 (Vehicle); mean±SEM. Significant difference compared to Vehicle, Ca+Lr+Bf, and anti-PD-1 alone, respectively, on Day 24** and Day 25** by 2-way ANOVA with Dunnett post hoc test. (E) The combination of Lr+Bf+anti-PD-1 antibody significantly increased tumor reduction in comparison to anti-PD-1 antibody alone. n=10 (Lr+Bf+anti-PD-1; anti-PD-1), n=10 (Vehicle); mean±SEM. Significant difference compared to Vehicle, Lr+As+Bf, and anti-PD-1 alone, respectively, on Day 24* and Day 25** by 2-way ANOVA with Dunnett post hoc test.

FIG. 25 depicts the effects of administration of a combination of Lactobacillus ruminis P167-B1a (Lr), Alistipes senegalensis P150-D12a (As) and Butyricimonas faecihominis P40-F2a (Bf) strains plus anti-PD-1 antibody on tumor-infiltrating immune cell populations in a murine CT26 colon tumor model. Trends for increased infiltration of (A) CD3⁺ T cells; (B) CD4⁺ T cells; (C) CD8⁺ T cells; and (D) Nkp46⁺ NK cells compared to administration of anti-PD-1 antibody only, as a frequency of non-necrotic cells in fixed tumor sections by immunohistochemistry (IHC). (E)-(J): Tumor-infiltrating immune cells were scored by targeted gene expression using Nanostring platform from RNA extracted from frozen tumor pieces. (E) T cell score based on expression of the following genes: Cd3d, Cd3e, Cd3g, Cd6, Sh2d1a and Trat1. (F) Th1 cell score based on expression of Tbx21. (G) Exhausted CD8 T cell score based on expression of the following genes: Cd244, Eomes, Lag3 and Ptger4. (H) NK CD56dim cell score based on expression of: Il21r, Kir3d11 and Kir3d12. (I) Cytotoxic T cell score based on expression of: Ctsw, Gzma, Gzmb, Klrb1, Klrd1, Klrk1, Nkg7 and Prf1. (J) CD45 score based on expression of Ptprc. *P<0.05, **P<0.01, ***P<0.001 and ****P<0.0001 by 1-way ANOVA with Dunnett's post hoc test compared to Vehicle or α-PD-1; mean+SEM.

FIG. 26 depicts the effects of administration of a combination of Collinsella ASMB P121-D5a (Ca), Lactobacillus ruminis P167-B1a (Lr) and Butyricimonas faecihominis P40-F2a (Bf) strains, with and without the addition of anti-PD-1 antibody, on tumor-infiltrating immune cell populations in a murine CT26 colon tumor model. Shown are trends for increased infiltration of: (A) CD3⁺ T cells; (B) CD4⁺ T cells; (C) CD8⁺ T cells; and (D) Nkp46⁺ NK cells, when compared to anti-PD-1 and significant increases compared to vehicle as a frequency of non-necrotic cells in fixed tumor sections by immunohistochemistry (IHC). (E)-(L): Tumor-infiltrating immune cells were scored by targeted gene expression using Nanostring platform from RNA extracted from frozen tumor pieces. (E) T cell score based on expression of the following genes: Cd3d, Cd3e, Cd3g, Cd6, Sh2d1a and Trat1. (F) Th1 cell score based on expression of Tbx21. (G) Exhausted CD8 T cell score based on expression of: Cd244, Eomes, Lag3 and Ptger4. (H) NK CD56dim cell score based on expression of: Il21r, Kir3d11, and Kir3d12. (I) CD45 score based on expression of Ptprc. (J) Neutrophil score based on expression of: Ceacam3, Csf3r, Fcgr4 and Fpr1. (K) Cytotoxic T cell score based on expression of: Ctsw, Gzma, Gzmb, Klrb1, Klrd1, Klrk1, Nkg7 and Prf1. (L) Dendritic cell score based on expression of: Ccl2, Cd209e and Hsd11b1. *P<0.05, **P<0.01, ***P<0.001 and ****P<0.0001 by 1-way ANOVA with Dunnett's post hoc test compared to Vehicle or α-PD-1; mean+SEM.

FIG. 27 depicts the effects of administration of a combination of Lactobacillus ruminis P167-B1a (Lr) and Butyricimonas faecihominis P40-F2a (Bf) strains plus anti-PD-1 antibody on tumor-infiltrating immune cell populations in a murine CT26 colon tumor model. Shown are trends for increased infiltration of: (A) CD3⁺ T cells; (B) CD4⁺ T cells; (C) CD8⁺ T cells; and (D) Nkp46⁺ NK cells compared to administration of anti-PD-1 alone, and significant increases compared to vehicle as a frequency of non-necrotic cells in fixed tumor sections by immunohistochemistry (IHC). (E)-(L): Tumor-infiltrating immune cells were scored by targeted gene expression using Nanostring platform from RNA extracted from frozen tumor pieces. (E) T cell score based on expression of: Cd3d, Cd3e, Cd3g, Cd6, Sh2d1a and Trat1. (F) Th1 cell score based on expression of Tbx21. (G) Exhausted CD8 T cell score based on expression of: Cd244, Eomes, Lag3, and Ptger4. (H) NK CD56dim cell score based on expression of: Il21r, Kir3d11, and Kir3d12. (I) CD45 score based on expression of Ptprc. (J) Neutrophil score based on expression of: Ceacam3, Csf3r, Fcgr4 and Fpr1. (K) Cytotoxic T cell score based on expression of: Ctsw, Gzma, Gzmb, Klrb1, Klrd1, Klrk1, Nkg7, and Prf1. (L) CD8 score based on expression of Cd8a and Cd8b1. *P<0.05, **P<0.01, ***P<0.001 and ****P<0.0001 by 1-way ANOVA with Dunnett's post hoc test compared to Vehicle or α-PD-1; mean+SEM.

FIG. 28 depicts the effects of administration of anti-PD-1 antibody with the combination of Lactobacillus ruminis P167-B1a (Lr) and Butyricimonas faecihominis P40-F2a (Bf) strains reconstituted from lyophilized stocks in a murine CT26 colon tumor model, in comparison to administration of anti-PD-1 antibody with the combination of Lr and Bf strains reconstituted from frozen stocks. n=10 (Lr+Bf+anti-PD-1; anti-PD-1), n=10 (Excipient); *P<0.05, **P<0.01, ***P<0.001, ****P<0.0001 2-way ANOVA with Fisher post hoc; mean+SEM.

FIG. 29 depicts the effects of administration of an anti-PD-1 antibody alone to: (A) a combination of Alistipes indistinctus and Butyricimonas faecihominis P40-F2a (Bf) strains and an anti-PD-1 antibody; (B) a combination of Bacteroides thetaiotaomicron and Bf strains and an anti-PD-1 antibody; and (C) a combination of Intestinimonas butyriciproducens and Bf strains and an anti-PD-1 antibody, in a murine CT26 colon tumor model. None of the 2-strain combinations plus anti-PD-1 antibody significantly increased tumor reduction compared to anti-PD-1 alone. n=10 (2-strain combo+anti-PD-1; anti-PD-1), n=10 (Vehicle). *P<0.05, **P<0.01, ***P<0.001, ****P<0.0001 by 2-way ANOVA with Fisher's LSD post hoc test; mean±SEM.

### DETAILED DESCRIPTION

### I. Bacterial Strains

A contemplated bacterial strain, for example, for use in a bacterial strain mixture, pharmaceutical composition or unit, or method provided herein, includes a Butyricimonas species strain. Exemplary Butyricimonas species include Butyricimonas faecihominis. Those of skill in the art will recognize that the genus Butyricimonas may undergo taxonomical reorganization. Thus, it is intended that contemplated Butyricimonas species include Butyricimonas species that have been renamed and/or reclassified, as well as those that may be later renamed and/or reclassified.

As used herein, the term “species” refers to a taxonomic entity as conventionally defined by genomic sequence and phenotypic characteristics. A “strain” is a particular instance of a species that has been isolated and purified according to conventional microbiological techniques. Bacterial species and/or strains described herein include those that are live and/or viable, as well as those that are killed, inactivated or attenuated. Additionally, bacterial species and/or strains described herein include vegetative forms and non-spore forming forms of bacteria.

In some embodiments, a bacterial strain of Butyricimonas comprises a 16S rRNA gene sequence having a certain % identity to a reference sequence. rRNA, 16S rDNA, 16S rRNA, 16S, 18S, 18S rRNA, and 18S rDNA refer to nucleic acids that are components of, or encode for, components of the ribosome. There are two subunits in the ribosome termed the small subunit (SSU) and large subunit (LSU). Ribosomal RNA genes (rDNA) and their complementary RNA sequences are widely used for determination of the evolutionary relationships among organisms as they are variable, yet sufficiently conserved to allow cross-organism molecular comparisons. 16S rDNA sequence of the 30S SSU can be used, in embodiments, for molecular-based taxonomic assignments of prokaryotes. For example, 16S sequences may be used for phylogenetic reconstruction, as they are general highly conserved but contain specific hypervariable regions that harbor sufficient nucleotide diversity to differentiate genera and species of most bacteria. Although 16S rDNA sequence data has been used to provide taxonomic classification, closely related bacterial strains that are classified within the same genus and species may exhibit distinct biological phenotypes.

Accordingly, a bacterial strain of Butyricimonas provided herein includes strains comprising a 16s rRNA gene sequence having a certain % identity to SEQ ID NO: 846. In some embodiments, the bacterial strain is a strain of the genus Butyricimonas comprising a 16s rRNA gene sequence with at least 94% sequence identity to the polynucleotide sequence of SEQ ID NO: 846. In some embodiments, the bacterial strain comprises a 16s rRNA gene sequence with at least about 94%, about 94.25%, about 94.5%, about 94.75%, about 95%, about 95.25%, about 95.5%, about 95.75%, about 96%, about 96.25%, about 96.5%, about 96.75%, about 97%, about 97.05%, about 97.1%, about 97.15%, about 97.2%, about 97.25%, about 97.3%, about 97.35%, about 97.4%, about 97.45%, about 97.5%, about 97.55%, about 97.6%, about 97.65%, about 97.7%, about 97.75%, about 97.8%, about 97.85%, about 97.9%, about 97.95%, about 98%, about 98.05%, about 98.1%, about 98.15%, about 98.2%, about 98.25%, about 98.3%, about 98.35%, about 98.4%, about 98.45%, about 98.5%, about 98.55%, about 98.6%, about 98.65%, about 98.7%, about 98.75%, about 98.8%, about 98.85%, about 98.9%, about 98.95%, about 99%, about 99.1%, about 99.2%, about 99.3%, about 99.4%, about 99.5%, about 99.6%, about 99.7%, about 99.8%, or about 99.9% identity to the polynucleotide sequence of SEQ ID NO: 846. In a particular embodiment, the bacterial strain comprises a 16s rRNA gene sequence identical to SEQ ID NO: 846. In some embodiments, the sequence identity referred to above is across at least about 70% of SEQ ID NO: 846. In other embodiments, the sequence identity referred to above is across at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% of SEQ ID NO: 846.

In some embodiments, the bacterial strain of Butyricimonas provided herein is a bacterial strain of Butyricimonas faecihominis. In some embodiments, the bacterial strain of Butyricimonas faecihominis comprises a genomic sequence (e.g., a whole genome sequence, or fragments or contigs thereof) having a certain % identity to one or more of SEQ ID NOs: 1-845. In some embodiments, a Butyricimonas faecihominis strain comprises a polynucleotide sequence selected from any one of SEQ ID NOs: 1-845, or a nucleotide sequence having at least about 65%, about 70%, about 75%, about 80%, about 85%, about 90%, about 91%, about 92%, about 93%, about 94%, about 95%, about 96%, about 97%, about 98%, or about 99% identity to a polynucleotide sequence selected from any one of SEQ ID NOs: 1-845. In some embodiments, the sequence identity referred to above is across at least about 70% of the bacterial genome. In other embodiments, the sequence identity referred to above is across at least about 75%, 80%, 85%, 90%, 95% or greater than 95% of the bacterial genome. In some embodiments, a Butyricimonas faecihominis strain genome may comprise the polynucleotide sequence of each of SEQ ID NOs: 1-845, or each of a polynucleotide sequence having at least about 65%, about 70%, about 75%, about 80%, about 85%, about 90%, about 91%, about 92%, about 93%, about 94%, about 95%, about 96%, about 97%, about 98%, or about 99% identity to the polynucleotide sequence of each of SEQ ID NOs: 1-845.

In some embodiments, a bacterial strain of Butyricimonas faecihominis comprises a whole genomic sequence having at least about 70% identity across at least 70% of its genome to the sum of all genomic contigs represented by SEQ ID NOs: 1-845. In some embodiments, the whole genomic sequence has at least about 75%, 80%, 85%, 90%, 95% or greater than 95% identity to the sum of all genomic contigs represented by SEQ ID NOs: 1-845. In some embodiments, the sequence identity referred to above is across at least 75%, 80%, 85%, 90%, 95% or greater than 95% of the whole genomic sequence of the bacterial strain. In some embodiments, a bacterial strain of Butyricimonas faecihominis comprises a whole genomic sequence comprising coding regions having at least about 70% identity across at least 70% of the total coding regions in its genome to the coding regions within the sum of all genomic contigs represented by SEQ ID NOs: 1-845. In some embodiments, the coding regions within the whole genomic sequence have at least about 75%, 80%, 85%, 90%, 95% or greater than 95% identity to the coding regions within the sum of all genomic contigs represented by SEQ ID NOs: 1-845. In some embodiments, the sequence identity referred to above is across at least 75%, 80%, 85%, 90%, 95% or greater than 95% of the coding regions within the whole genomic sequence of the bacterial strain.

The identity of a bacterial strain of Butyricimonas may be determined by sequence analysis, for example, of the 16s rRNA gene sequence or a genomic sequence (e.g., a whole genome sequence, or fragments or contigs thereof) of the bacterial strain, using any sequencing methods known in the art, including, for example, Sanger sequencing. An example of a sequencing technology useful for identifying strains of Butyricimonas is the Illumina platform. The Illumina platform is based on amplification of DNA on a solid surface (e.g., flow cell) using fold-back PCR and anchored primers (e.g., capture oligonucleotides). For sequencing with the Illumina platform, bacterial DNA is fragmented, and adapters are added to terminal ends of the fragments. DNA fragments are attached to the surface of flow cell channels by capturing oligonucleotides which are capable of hybridizing to the adapter ends of the fragments. The DNA fragments are then extended and bridge amplified. After multiple cycles of solid-phase amplification followed by denaturation, an array of millions of spatially immobilized nucleic acid clusters or colonies of single-stranded nucleic acids are generated. Each cluster may include approximately hundreds to a thousand copies of single-stranded DNA molecules of the same template. The Illumina platform uses a sequencing-by-synthesis method where sequencing nucleotides comprising detectable labels (e.g., fluorophores) are added successively to a free 3′ hydroxyl group. After nucleotide incorporation, a laser light of a wavelength specific for the labeled nucleotides can be used to excite the labels. An image is captured and the identity of the nucleotide base is recorded. These steps can be repeated to sequence the rest of the bases. Sequencing according to this technology is described in, for example, U.S. Patent Publication Application Nos. 2011/0009278, 2007/0014362, 2006/0024681, 2006/0292611, and U.S. Pat. Nos. 7,960,120, 7,835,871, 7,232,656, and 7,115,200. Another example of a sequencing technology useful for identifying strains of Butyricimonas is SOLiD technology by Applied Biosystems from Life Technologies Corporation (Carlsbad, Calif.). In SOLiD sequencing, bacterial DNA may be sheared into fragments, and adapters may be attached to the terminal ends of the fragments to generate a library. Clonal bead populations may be prepared in microreactors containing template, PCR reaction components, beads, and primers. After PCR, the templates can be denatured, and bead enrichment can be performed to separate beads with extended primers. Templates on the selected beads undergo a 3′ modification to allow covalent attachment to the slide. The sequence can be determined by sequential hybridization and ligation with several primers. A set of four fluorescently labeled di-base probes compete for ligation to the sequencing primer. Multiple cycles of ligation, detection, and cleavage are performed with the number of cycles determining the eventual read length. Another example of a sequencing technology useful for identifying strains of Butyricimonas is Ion Torrent sequencing. In this technology, bacterial DNA is sheared into fragments, and oligonucleotide adapters are then ligated to the terminal ends of the fragments. The fragments are then attached to a surface, and each base in the fragments is resolvable by measuring the H⁺ ions released during base incorporation. This technology is described in, for example, U.S. Patent Publication Application Nos. 2009/0026082, 2009/0127589, 2010/0035252, 2010/0137143, and 2010/0188073.

Upon obtaining a polynucleotide sequence of a bacterial strain (e.g., 16s rRNA gene sequence or genomic sequence), sequence identity with a polynucleotide sequence of a Butyricimonas strain may be determined in various ways that are within the skill in the art, e.g., using publicly available computer software such as BLAST, BLAST-2, BLAT (BLAST-like alignment tool), ALIGN or Megalign (DNASTAR) software. BLAST (Basic Local Alignment Search Tool) analysis using the algorithm employed by the programs blastp, blastn, blastx, tblastn and tblastx (Karlin et al., PROC. NATL. ACAD. SCI. USA 87:2264-2268 (1990); Altschul, J. MOL. EVOL. 36, 290-300 (1993); Altschul et al., NUCLEIC ACIDS RES. 25:3389-3402 (1997)) are tailored for sequence similarity searching. For a discussion of basic issues in searching sequence databases see Altschul et al., NATURE GENETICS 6:119-129 (1994). Those skilled in the art can determine appropriate parameters for measuring alignment, including any algorithms needed to achieve maximal alignment over the full length of the sequences being compared. The search parameters for histogram, descriptions, alignments, expect value (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919). Four blastn parameters may be adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; —W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and —Z, final X dropoff value for gapped alignment (in bits): 50 for blastn, 25 for others). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In a particular embodiment, a bacterial strain of Butyricimonas useful for the compositions and methods provided herein is Butyricimonas faecihominis strain P40-F2a. A deposit of Butyricimonas faecihominis strain P40-F2a was made to DSMZ (Deutsche Sammlung von Mikroorganismen and Zellkulturen GmbH, Inhoffenstraβe 7B, 38124 Brunswick, Germany) under the Budapest Treaty on the International Recognition of the Deposit of Microorganisms for the Purposes of Patent Procedure on Jan. 20, 2020. This deposit was accorded accession number DSM 33411. The 16s rRNA gene sequence of Butyricimonas faecihominis strain P40-F2a is provided herein as SEQ ID NO: 846, and genomic sequences of Butyricimonas faecihominis strain P40-F2a are provided herein as SEQ ID NOs: 1-845. In another particular embodiment, a bacterial strain of Butyricimonas useful for the compositions and methods provided herein is Butyricimonas faecihominis strain 180-3^{T} (=JCM 18676^{T}=CCUG65562^{T}). See Sakamoto et al., International Journal of Systematic and Evolutionary Microbiology 64, 2992-2997 (2014). In another particular embodiment, a bacterial strain of Butyricimonas useful for the compositions and methods provided herein is Butyricimonas faecihominis strain 30A1 (GenBank assembly accession: GCA_003851945.2; RefSeq assembly accession: GCF_003851945.2).

Additional bacterial strains of Butyricimonas provided herein include Butyricimonas strains having a DNA-DNA hybridization (DDH)) value of equal to or greater than about 70% with Butyricimonas faecihominis strain P40-F2a. In particular embodiments, the Butyricimonas faecihominis strain is one having greater than about 70%, about 75%, about 80%, about 85%, about 90%, about 95%, about 96%, about 97%, about 98%, or about 99% DNA-DNA hybridization with Butyricimonas faecihominis strain P40-F2a, or any range between any of the above values. Any method for determining DNA-DNA hybridization values known in the art may be used to assess the degree of DNA-DNA hybridization, including but not limited to the spectrophotometric method for determining renaturation rates described by De Ley et al. (J Biochem 12 133-142 (1970)), slightly modified in hybridization temperature (Gavini et al., Ecology in Health and Disease 12 40-45 (2001)); and those described by Grimont et al., Curr Microbiol 4, 325-330 (1980) and Rossello-Mora, Molecular Identification, Systematics and Population Structure of Prokaryotes pp. 23-50 (2006). In some embodiments, the degree of DNA-DNA hybridization is determined by digital DNA-DNA hybridization (dDDH) analysis, for example, using the Genome-to-Genome Distance Calculator online tool (see Meier-Kolthoff et al., BMC Bioinformatics 14:60 (2013)). In particular embodiments, the Butyricimonas strain is one having a DDH or dDDH value of equal to or greater than about 70% with Butyricimonas faecihominis strain P40-F2a. In some embodiments, the DDH or dDDH value is greater than about 75%, about 80%, about 85%, about 90%, about 95%, about 96%, about 97%, about 98%, or about 99% with Butyricimonas faecihominis strain P40-F2a, or any range between any of the above values.

Additional bacterial strains of Butyricimonas provided herein include Butyricimonas strains having equal to or greater than 95% average nucleotide identity (ANI) with Butyricimonas faecihominis strain P40-F2a. In some embodiments, the ANI is equal to or greater than about 95%, about 95.5%, about 96%, about 96.5%, about 97%, about 97.5%, about 98%, about 98.5%, about 99%, about 99.5% or 100% with Butyricimonas faecihominis strain P40-F2a, or any range between any of the above values. The average nucleotide identity (ANI) of the shared genes between two strains is known to be a robust means to compare genetic relatedness among strains, and that ANI values of ˜95% correspond to the 70% DNA-DNA hybridization standard for defining a species. See, e.g., Konstantinidis and Tiedje, Proc Natl Acad Sci USA, 102(7):2567-72 (2005); Goris et al., Int Syst Evol Microbiol. 57(Pt 1):81-91 (2007); and Jain et al., Nat Commun. 9(1):5114 (2018). In some embodiments, the ANI between two bacterial genomes is calculated from pair-wise comparisons of all sequences shared between any two strains and can be determined, for example, using any of a number of publicly available ANI tools, including but not limited to OrthoANI with usearch (Yoon et al. Antonie van Leeuwenhoek 110:1281-1286 (2017)); ANI Calculator, JSpecies (Richter and Rossello-Mora, Proc Natl Acad Sci USA 106:19126-19131 (2009)); and JSpeciesWS (Richter et al., Bioinformatics 32:929-931 (2016)). Other methods for determining the ANI of two genomes are known in the art. See, e.g., Konstantinidis, K. T. and Tiedje, J. M., Proc. Natl. Acad. Sci. U.S.A., 102: 2567-2572 (2005); Varghese et al., Nucleic Acids Research, 43(14):6761-6771 (2015); and Jain et al., Nat Commun. 9(1):5114 (2018). In a particular embodiment, the ANI between two bacterial genomes can be determined using an alignment-based method, for example, by averaging the nucleotide identity of orthologous genes identified as bidirectional best hits (BBHs). Protein-coding genes of a first genome (Genome A) and second genome (Genome B) are compared at the nucleotide level using a similarity search tool, for example, NSimScan (Novichkov et al., Bioinformatics 32(15): 2380-23811 (2016). The results are then filtered to retain only the BBHs that display at least 70% sequence identity over at least 70% of the length of the shorter sequence in each BBH pair. The ANI of Genome A to Genome B is defined as the sum of the percent identity times the alignment length for all BBHs, divided by the sum of the lengths of the BBH genes. In another particular embodiment, the ANI between two bacterial genomes can be determined using an alignment-free method, for example, FastANI, which uses alignment-free approximate sequence mapping to assess genomic relatedness. See Jain et al., Nat Commun. 9(1):5114 (2018). FastANI has been demonstrated to reveal clear genetic discontinuity between species, with 99.8% of the total 8 billion genome pairs analyzed conforming to >95% intra-species and <83% inter-species ANI values. Accordingly, in some embodiments, a bacterial strain having a genome with equal to or greater than 95% average nucleotide identity (ANI) with the genome of Butyricimonas faecihominis strain P40-F2a is identified as a bacterial strain of the species Butyricimonas faecihominis.

Additional bacterial strains of Butyricimonas provided herein include Butyricimonas strains having equal to or greater than 60% alignment fraction (AF) with Butyricimonas faecihominis strain P40-F2a. In some embodiments, the AF is equal to or greater than about 65%, about 70%, about 75%, about 80%, about 85%, about 90%, about 95%, or 100% with Butyricimonas faecihominis strain P40-F2a, or any range between any of the above values. In some embodiments, the AF is computed by dividing the sum of the lengths of all BBH genes by the sum of the length of all the genes in Genome A. This computation is performed separately in both directions: from Genome A to genome B and from Genome B to Genome A.

In a particular embodiment, a Butyricimonas strain comprises a genome having equal to or greater than about 95% ANI and equal to or greater than 60% AF with the genome of Butyricimonas faecihominis strain P40-F2a. In another particular embodiment, a Butyricimonas strain comprises a genome having equal to or greater than about 96.5% ANI and equal to or greater than 60% AF with the genome of Butyricimonas faecihominis strain P40-F2a.

Additional bacterial strains of Butyricimonas provided herein include Butyricimonas strains that having the same or approximately the same genome characteristics as Butyricimonas faecihominis strain P40-F2a. Such genome characteristics can include, for example, genome size, G+C content, number of coding sequences, and number of tRNAs. In some embodiments, the Butyricimonas strain comprises a genome of about 4.7 to about 4.9 Mb in size. In some embodiments, the Butyricimonas strain comprises a genome of about 4.71, 4.72, 4.73, 4.74, 4.75, 4.76, 4.77, 4.78, 4.79, 4.80, 4.81, 4.82, 4.83, 4.84, 4.85, 4.86, 4.87, 4.88, 4.89 or about 4.9 Mb in size. In a particular embodiment, the Butyricimonas strain comprises a genome of about 4.85 Mb in size. In some embodiments, the Butyricimonas strain comprises a genome that comprises about 3600 to 4000 coding sequences. In some embodiments, the Butyricimonas strain comprises a genome that comprises about 3650 to 3950 coding sequences. In some embodiments, the Butyricimonas strain comprises a genome that comprises about 3660, 3670, 3680, 3690, 3700, 3710, 3720, 3730, 3740, 3750, 3760, 3770, 3780, 3790, 3800, 3810, 3820, 3830, 3840, 3850, 3860, 3870, 3880, 3890, 3900, 3910, 3920, 3930, or 3940 coding sequences. In a particular embodiment, the Butyricimonas strain comprises a genome that comprises about 3904 coding sequences. In some embodiments, the Butyricimonas strain comprises a genome that comprises about 40 to 60 tRNA sequences. In some embodiments, the Butyricimonas strain comprises a genome that comprises about 41, 42, 43, 44, 45, 46, 47, 48, 49, 50, 51, 52, 53, 54, 55, 56, 57, 58, 59 or 60 tRNAs. In a particular embodiment, the Butyricimonas strain comprises a genome that comprises about 49 tRNAs. In some embodiments, the Butyricimonas strain comprises a genome that has a G+C content of about 35% to about 55%. In some embodiments, the Butyricimonas strain comprises a genome that has a G+C content of about 40% to about 50%. In some embodiments, the Butyricimonas strain comprises a genome that has a G+C content of about 40.0%, 40.5%, 41.0%, 41.5%, 42.0%, 42.5%, 43.0%, 43.5%, 44.0%, 44.5%, 45.0%, 45.5%, 46.0%, 46.5%, 47.0%, 47.5%, 48.0%, 48.5%, 49.0%, 49.5%, or about 50%. In a particular embodiment, the Butyricimonas strain comprises a genome that has a G+C content of about 43.47%.

Additional bacterial strains of Butyricimonas provided herein include Butyricimonas strains that provide the same or approximately the same pattern as Butyricimonas faecihominis strain P40-F2a when analyzed, for example, by DNA fingerprinting techniques. Any DNA fingerprinting technique known in the art may be used to identify strains of Butyricimonas, including but not limited to, Pulsed Field Gel Electrophoresis (PFGE), ribotyping, Randomly Amplified Polymorphic DNA (RAPD), Amplified Fragment Length Polymorphism (AFLP), Amplified Ribosomal DNA Restriction Analysis (ARDRA), rep-PCR (repetitive element primed PCR, directed to naturally occurring, highly conserved, repetitive DNA sequences, present in multiple copies in the genomes) including Repetitive Extragenic Palindromic PCR (REP-PCR), Enterobacterial Repetitive Intergenic Consensus Sequences-PCR (ERIC-PCR), BOX-PCR (derived from the boxA element), (GTG)₅-PCR, Triplicate Arbitrary Primed PCR (TAP-PCR), Multi-Locus Sequence Analysis (MLSA), Multi-Locus Sequence Typing (MLST), Multiple Locus Variable-number Tandem Repeat Analysis (MLVA) and DNA microarray-based genotyping techniques.

Additional bacterial strains of Butyricimonas provided herein include Butyricimonas strains showing phenotypic similarity to Butyricimonas faecihominis strain P40-F2a. Phenotypic similarity can be based on, for example, cell shape and size, colony morphology (e.g. size, color and odor of plate colonies), Gram staining, biochemical tests, pH and temperature optima, sugar fermentation, metabolic capabilities (e.g. catalase and/or oxidase negative), chemotaxonomic analysis (e.g. polar lipid and lipoquinone composition; see Tindall et al., Int J Syst Evol Microbiol 58, 1737-1745 (2008)) and/or fatty acid methyl ester (FAME) analysis.

Additional bacterial strains of Butyricimonas provided herein include Butyricimonas strains showing carbon utilization similar to Butyricimonas faecihominis strain P40-F2a. In some embodiments, the Butyricimonas bacterial strain is capable of utilizing one or more carbon sources selected from the group consisting of α-D-Glucose, D-Mannose, α-D-Lactose, Glycerol, D-Galactose, N-Acetyl-D-glucosamine, D,L-α-Glycerol-Phosphate, D-Glucosamine, N-Acetyl-D-Galactosamine, N-Acetyl-Neuraminic Acid, α-Keto-Valeric Acid, and Melibionic Acid.

A contemplated bacterial strain, bacterial strain mixture, or composition may be characterized as having an effect on gene product production, e.g., IFN-γ, IL-1β, IL-6, IL-12p40, IL-12p70, IL-23, IL-27, TNF, and/or TRAIL production, in a cell, tissue, or subject, e.g., an immune cell, e.g., a macrophage (e.g., a THP-1 macrophage) or PBMC (including lymphocytes (T cells, B cells, NK cells) and monocytes). In vivo, major sources of cytokines include T helper cells, monocytes, macrophages and dendritic cells, however myriad immune effector cell types are capable of producing cytokines in certain contexts including B cells, cytotoxic T cells, NK cells, mast cells, and granulocytes like neutrophils and eosinophils. Gene product production, e.g., IFN-γ, IL-1β, IL-6, IL-12p40, IL-12p70, IL-23, IL-27, TNF, and/or TRAIL production, in a macrophage may, for example, be assayed as follows. THP-1 human macrophages are made by culturing the THP-1 human monocyte cell line with phorbol 12-myristate 13-acetate (PMA) for 24 hours, optionally followed by IL-4 and IL-13 as described previously (Genin et al., BMC Cancer 15:577 (2015)). A bacterial strain, bacterial strain mixture, or composition is incubated with THP-1 macrophages in the presence of lipopolysaccharide (LPS) for 24 hours. Gene product production is assessed by measuring the concentration of the gene product, e.g., IFN-γ, IL-1β, IL-6, IL-12p40, IL-12p′70, IL-23, IL-27, TNF, and/or TRAIL, in the cell culture supernatant by ELISA. Gene product production may also be assayed as described in Sudhakaran et al., Genes Nutr., 8(6): 637-48 (2013). Gene product production, e.g., IFN-γ, IL-1β, IL-6, IL-12p40, IL-12p70, IL-23, IL-27, TNF, and/or TRAIL production, in a PBMC may, for example, be assayed as follows. Primary PBMCs are isolated from blood samples of donors using a percoll gradient (Sim et al., J. Vis. Exp. (112), e54128 (2016)). A bacterial strain, bacterial strain mixture, or composition is incubated with PBMCs for 24 hours. Gene product production is assessed by measuring the concentration of the gene product, e.g., IFN-γ, IL-1β, IL-6, IL-12p40, IL-12p′70, IL-23, IL-27, TNF, and/or TRAIL, in the cell culture supernatant by ELISA.

In some embodiments, a Butyricimonas (e.g., Butyricimonas faecihominis) bacterial strain (or a bacterial strain mixture or composition comprising the Butyricimonas bacterial strain) increases, or is capable of increasing, production of at least one pro-inflammatory gene product, e.g., a pro-inflammatory cytokine or chemokine, in a cell, tissue, or subject. Exemplary pro-inflammatory gene products include IFN-γ, IL-1β, IL-6, IL-12p40, IL-12p70, IL-23, IL-27, TNF, and TRAIL. For example, in some embodiments, a Butyricimonas (e.g., Butyricimonas faecihominis) bacterial strain provided herein (or a composition or bacterial strain mixture comprising the Butyricimonas bacterial strain) increases, or is capable of increasing, expression of one or more of IFN-γ, IL-1β, IL-6, IL-12p40, IL-12p70, IL-23, IL-27, TNF, and TRAIL in a cell, tissue, or subject. In some embodiments, the increased production of a pro-inflammatory gene product, e.g., IFN-γ, IL-1β, IL-6, IL-12p40, IL-12p′70, IL-23, IL-27, TNF, and/or TRAIL, occurs in a human cell, e.g., a THP-1 macrophage, monocyte, PBMC or moDC. For example, contacting a human cell, e.g., a THP-1 macrophage, PBMC or moDC with the Butyricimonas (e.g., Butyricimonas faecihominis) bacterial strain (or the composition or bacterial strain mixture comprising the Butyricimonas bacterial strain), e.g., by culturing the human cell with the Butyricimonas bacterial strain (or the composition or bacterial strain mixture comprising the Butyricimonas bacterial strain) increases production of IFN-γ, IL-1β, IL-6, IL-12p40, IL-12p70, IL-23, IL-27, TNF, and/or TRAIL in the cell by at least about 10%, at least about 20%, at least about 30%, at least about 40%, at least about 50%, at least about 75%, at least about 100%, at least about 200%, at least about 300%, at least about 400%, at least about 500%, at least about 750%, at least about 1000%, from about 10% to about 20%, from about 10% to about 50%, from about 10% to about 100%, from about 10% to about 200%, from about 10% to about 500%, from about 10% to about 1000%, from about 20% to about 50%, from about 20% to about 100%, from about 20% to about 200%, from about 20% to about 500%, from about 20% to about 1000%, from about 50% to about 100%, from about 50% to about 200%, from about 50% to about 500%, from about 50% to about 1000%, from about 100% to about 200%, from about 100% to about 500%, from about 100% to about 1000%, from about 200% to about 500%, from about 200% to about 1000%, or from about 500% to about 1000%, relative to a cell (e.g., of the same cell type) that was not contacted, e.g., cultured, with the Butyricimonas bacterial strain (or the composition or bacterial strain mixture comprising the Butyricimonas bacterial strain). In some embodiments, the contacting of the human cell with the Butyricimonas (e.g., Butyricimonas faecihominis) strain (or the composition or bacterial strain mixture comprising the Butyricimonas bacterial strain) occurs in vitro. In other embodiments, the contacting of the human cell with the Butyricimonas (e.g., Butyricimonas faecihominis) strain (or the composition or bacterial strain mixture comprising the Butyricimonas bacterial strain) occurs in vivo.

In some embodiments, a Butyricimonas (e.g., Butyricimonas faecihominis) bacterial strain (or a bacterial strain mixture or composition comprising the Butyricimonas bacterial strain) increases, or is capable of increasing, infiltration of T cells into a tumor (e.g., a tumor in a subject). In some embodiments, the T cells are CD3+ T cells. In some embodiments, the T cells are CD8+ T cells. In some embodiments, contacting a tumor with the Butyricimonas (e.g., Butyricimonas faecihominis) bacterial strain (or the composition or bacterial strain mixture comprising the Butyricimonas bacterial strain), e.g., by administering the Butyricimonas bacterial strain (or the composition or bacterial strain mixture comprising the Butyricimonas bacterial strain) to a subject with the tumor, increases infiltration of T cells into the tumor by at least about 10%, at least about 20%, at least about 30%, at least about 40%, at least about 50%, at least about 75%, at least about 100%, at least about 200%, at least about 300%, at least about 400%, at least about 500%, at least about 750%, at least about 1000%, from about 10% to about 20%, from about 10% to about 50%, from about 10% to about 100%, from about 10% to about 200%, from about 10% to about 500%, from about 10% to about 1000%, from about 20% to about 50%, from about 20% to about 100%, from about 20% to about 200%, from about 20% to about 500%, from about 20% to about 1000%, from about 50% to about 100%, from about 50% to about 200%, from about 50% to about 500%, from about 50% to about 1000%, from about 100% to about 200%, from about 100% to about 500%, from about 100% to about 1000%, from about 200% to about 500%, from about 200% to about 1000%, or from about 500% to about 1000%, relative to a tumor (e.g., of the same tumor type) that was not contacted with the Butyricimonas bacterial strain (or the composition or bacterial strain mixture comprising the Butyricimonas bacterial strain). In some embodiments, said contacting further comprises contacting the tumor with a checkpoint inhibitor, for example, a checkpoint inhibitor antibody.

Also provided herein are strains of the species Lactobacillus ruminis, for example, a strain referred to herein as Lactobacillus ruminis strain P167-B1a, and compositions, for example, pharmaceutical compositions, comprising such strains.

A bacterial strain of the species Lactobacillus ruminis provided herein includes strains comprising a 16s rRNA gene sequence having a certain % identity to SEQ ID NO: 847. In some embodiments, the bacterial strain comprises a 16s rRNA gene sequence with at least about 98.00%, about 98.05%, about 98.1%, about 98.15%, about 98.2%, about 98.25%, about 98.3%, about 98.35%, about 98.4%, about 98.45%, about 98.5%, about 98.55%, about 98.6%, about 98.65%, about 98.7%, about 98.75%, about 98.80%, about 98.85%, about 99%, about 99.1%, about 99.2%, about 99.3%, about 99.4%, about 99.5%, about 99.6%, about 99.7%, about 99.8%, or about 99.9% identity to the polynucleotide sequence of SEQ ID NO: 847. In a particular embodiment, the bacterial strain comprises a 16s rRNA gene sequence identical to SEQ ID NO: 847. In some embodiments, the sequence identity referred to above is across at least about 70% of SEQ ID NO: 847. In other embodiments, the sequence identity referred to above is across at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of SEQ ID NO: 847.

In a particular embodiment, a bacterial strain of Lactobacillus ruminis provided herein is Lactobacillus ruminis strain P167-B1a. A deposit of Lactobacillus ruminis strain P167-B1a was made to DSMZ (Deutsche Sammlung von Mikroorganismen and Zellkulturen GmbH, Inhoffenstraβe 7B, 38124 Brunswick, Germany) under the Budapest Treaty on the International Recognition of the Deposit of Microorganisms for the Purposes of Patent Procedure on Mar. 6, 2020. This deposit was accorded accession number DSM 33536. The 16S rDNA sequence of Lactobacillus ruminis strain P167-B1a is provided as SEQ ID NO: 847.

Additional bacterial strains of the species Lactobacillus ruminis provided herein include Lactobacillus ruminis strains having a DNA-DNA hybridization (DDH) value of equal to or greater than about 70% with Lactobacillus ruminis strain P167-B1a. In particular embodiments, the Lactobacillus ruminis strain is one having greater than about 70%, about 75%, about 80%, about 85%, about 90%, about 95%, about 98%, or about 99% DNA-DNA hybridization with Lactobacillus ruminis strain P167-B1a, or any range between any of the above values. In particular embodiments, the Lactobacillus ruminis strain is one having a DDH or dDDH value of equal to or greater than about 70% with Lactobacillus ruminis strain P167-B1a. In some embodiments, the DDH or dDDH value is greater than about 75%, about 80%, about 85%, about 90%, about 95%, about 98%, or about 99% with Lactobacillus ruminis strain P167-B1a, or any range between any of the above values.

Additional bacterial strains of the species Lactobacillus ruminis provided herein include Lactobacillus ruminis strains having equal to or greater than 95% average nucleotide identity (ANI) with Lactobacillus ruminis strain P127-A10a. In some embodiments, the ANI is equal to or greater than about 95%, about 95.5%, about 96%, about 96.5%, about 97%, about 97.5%, about 98%, about 98.5%, about 99%, about 99.5%, or 100% with Lactobacillus ruminis strain P167-B1a, or any range between any of the above values.

Additional bacterial strains of the species Lactobacillus ruminis provided herein include Lactobacillus ruminis strains having equal to or greater than 60% alignment fraction (AF) with Lactobacillus ruminis strain P167-B1a. In some embodiments, the AF is equal to or greater than about 65%, about 70%, about 75%, about 80%, about 85%, about 90%, about 95%, or 100% with Lactobacillus ruminis strain P167-B1a, or any range between any of the above values. In some embodiments, the AF is computed by dividing the sum of the lengths of all BBH genes by the sum of the length of all the genes in Genome A. This computation is performed separately in both directions: from Genome A to genome B and from Genome B to Genome A.

In a particular embodiment, an Lactobacillus ruminis strain comprises a genome having equal to or greater than about 95% ANI and equal to or greater than 60% AF with the genome of Lactobacillus ruminis strain P167-B1a. In another particular embodiment, an Lactobacillus ruminis strain comprises a genome having equal to or greater than about 96.5% ANI and equal to or greater than 60% AF with the genome of Lactobacillus ruminis strain P167-B1a.

Those of skill in the art will recognize that the genus Lactobacillus may undergo taxonomical reorganization. Thus, it is intended that a contemplated Lactobacillus species include Lactobacillus species that have been renamed and/or reclassified, as well as those that may be later renamed and/or reclassified. For example, contemplated strains of Lactobacillus ruminis includes those reclassified as Ligilactobacillus ruminis (see e.g., Zheng et al., Int. J. Syst. Evol. Microbiol. 2020; 70:2782-2858).

In some embodiments, a bacterial strain mixture or composition comprising the Butyricimonas bacterial strain (e.g., a bacterial strain mixture or composition that increases, or is capable of increasing, infiltration of T cells into a tumor) may comprise Butyricimonas faecihominis and one or more non-Butyricimonas bacterial species. In some embodiments, the one or more non-Butyricimonas bacterial species includes a member of the genus Collinsella, for example, Collinsella ASMB P121-D5a. Collinsella ASMB P121-D5a is described in U.S. Provisional Application No. 62/955,957 filed Dec. 31, 2019 and U.S. Provisional Application No. 63/035,132 filed Jun. 5, 2020. A deposit of Collinsella ASMB strain P121-D5a was made to DSMZ (Deutsche Sammlung von Mikroorganismen and Zellkulturen GmbH, Inhoffenstraβe 7B, 38124 Brunswick, Germany) under the Budapest Treaty on the International Recognition of the Deposit of Microorganisms for the Purposes of Patent Procedure on Sep. 20, 2019. This deposit was accorded accession number DSM 33276.

In some embodiments, the one or more non-Butyricimonas bacterial species includes a member of the genus Alistipes, for example, Alistipes senegalensis. In a particular embodiment, the Alistipes senegalensis is Alistipes senegalensis strain P150-D12a. Alistipes senegalensis P150-D12a is described in U.S. Provisional Application No. 62/959,280 filed Jan. 10, 2020 and U.S. Provisional Application No. 63/035,135 filed Jun. 5, 2020. A deposit of Alistipes senegalensis strain P150-D12a was made to DSMZ (Deutsche Sammlung von Mikroorganismen and Zellkulturen GmbH, Inhoffenstraβe 7B, 38124 Brunswick, Germany) under the Budapest Treaty on the International Recognition of the Deposit of Microorganisms for the Purposes of Patent Procedure on Dec. 13, 2019. This deposit was accorded accession number DSM 33382.

In some embodiments, the one or more non-Butyricimonas bacterial species includes a member of the genus Lactobacillus, for example, Lactobacillus ruminis. In a particular embodiment, the Lactobacillus ruminis is Lactobacillus ruminis strain P167-B1a. A deposit of Lactobacillus ruminis strain P167-B1a was made to DSMZ (Deutsche Sammlung von Mikroorganismen and Zellkulturen GmbH, Inhoffenstraβe 7B, 38124 Brunswick, Germany) under the Budapest Treaty on the International Recognition of the Deposit of Microorganisms for the Purposes of Patent Procedure on Mar. 6, 2020. This deposit was accorded accession number DSM 33536.

In some embodiments, the one or more non-Butyricimonas bacterial species includes (i) a member of the genus Collinsella, for example, Collinsella ASMB P121-D5a; and/or (ii) a member of the genus Alistipes, for example, Alistipes senegalensis, for example, Alistipes senegalensis strain P150-D12a; and/or a member of the genus Lactobacillus, for example, Lactobacillus ruminis, for example, Lactobacillus ruminis strain P167-B1a. In some embodiments a bacterial strain mixture or composition comprising a Butyricimonas (e.g., Butyricimonas faecihominis) bacterial strain and optionally, one or more non-Butyricimonas bacterial species (e.g., a Collinsella, Alistipes or Lactobacillus bacterial strain) also comprises an immune checkpoint inhibitor. As used herein, an “immune checkpoint inhibitor” refers to a molecule, such as e.g., a small molecule, a soluble receptor, or an antibody, which targets an immune checkpoint and blocks the function of said immune checkpoint. More specifically, an “immune checkpoint inhibitor” as used herein is a molecule, such as e.g., a small molecule, a soluble receptor, or an antibody, that is capable of inhibiting or otherwise decreasing one or more of the biological activities of an immune checkpoint. In some embodiments, an inhibitor of an immune checkpoint protein (e.g., an antagonistic antibody) can, for example, act by inhibiting or otherwise decreasing the activation and/or cell signaling pathways of the cell expressing said immune checkpoint protein (e.g., a T cell), thereby inhibiting a biological activity of the cell relative to the biological activity in the absence of the antagonist. Example of immune checkpoint inhibitors include small molecule drugs, soluble receptors, and antibodies. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist. In certain embodiments, the checkpoint inhibitor is a PD-1 or PD-L1 inhibitor. Exemplary PD-1/PD-L1 based immune checkpoint inhibitors include antibody-based therapeutics.

The present disclosure encompasses derivatives of the disclosed bacterial strains. The term “derivative” includes daughter strains (progeny) or stains cultured (sub-cloned) from the original but modified in some way (including at the genetic level), without negatively altering a biological activity of the strain.

### II. Compositions Comprising a Butyricimonas Strain

In another aspect, provided herein are compositions, for example pharmaceutical compositions, comprising a bacterial strain of Butyricimonas (e.g., Butyricimonas faecihominis). In some embodiments, the compositions comprise one or more bacterial strains, including one or more bacterial strains of Butyricimonas. In some embodiments, a composition provided herein comprises a bacterial strain of Butyricimonas faecihominis and does not comprise any other strains or species of bacteria. In other embodiments, the composition comprises a bacterial strain of Butyricimonas faecihominis and at least one or more additional strains or species of bacteria. In some embodiments, the at least one additional strain or species of bacteria in the composition is a bacterial strain of the genus Butyricimonas. For example, the composition may comprise an additional strain of Butyricimonas faecihominis and/or one or more strains of a Butyricimonas species that is not Butyricimonas faecihominis. Exemplary additional Butyricimonas species include Butyricimonas synergistica, Butyricimonas faecalis, Butyricimonas virosa and Butyricimonas paravirosa. In other embodiments, the composition may comprise Butyricimonas faecihominis and one or more non-Butyricimonas bacterial species. In some embodiments, the one or more non-Butyricimonas bacterial species includes Collinsella ASMB P121-D5a. In some embodiments, the one or more non-Butyricimonas bacterial species includes an Alistipes species, for example, Alistipes senegalensis. In a particular embodiment, the Alistipes senegalensis is Alistipes senegalensis strain P150-D12a. In some embodiments, the one or more non-Butyricimonas bacterial species includes (i) a member of the genus Collinsella, for example, Collinsella ASMB P121-D5a, and (ii) a member of the genus Alistipes, for example, Alistipes senegalensis, for example, Alistipes senegalensis strain P150-D12a. In yet other embodiments, the composition may comprise a Butyricimonas strain selected from Butyricimonas synergistica, Butyricimonas faecalis, Butyricimonas virosa and Butyricimonas paravirosa, and one or more non-Butyricimonas bacterial species.

A contemplated composition or bacterial strain mixture may, e.g., comprise or consist essentially of 2, 3, 4, 5, 6, 7, 8, 9, 10, or more than 10 bacterial strains. In some embodiments, one or more strains of the composition or bacterial strain mixture are vegetative. In some embodiments, all the strains of the composition or bacterial strain mixture are vegetative. For example, in certain embodiments, a disclosed composition or bacterial strain mixture comprises or consists essentially of 2 to 10, 2 to 9, 2 to 8, 2 to 7, 2 to 6, 2 to 5, 2 to 4, or 2 to 3 bacterial strains; or, for example, may comprise or consist essentially of 3 to 10, 3 to 9, 3 to 8, 3 to 7, 3 to 6, 3 to 5 or 3 to 4 bacterial strains; or, for example, may comprise or consist essentially of 4 to 10, 4 to 9, 4 to 8, 4 to 7, 4 to 6 or 4 to 5 bacterial strains; or, for example, may comprise or consist essentially of 5 to 10, 5 to 9, 5 to 8, 5 to 7, 5 to 6, 6 to 10, 6 to 9, 6 to 8, 6 to 7, 7 to 10, 7 to 9, or 7 to 8 bacterial strains; or, for example, may comprise or consist essentially of 8 to 10 or 8 to 9 bacterial strains. In some embodiments, a disclosed composition or bacterial strain mixture comprises or consists essentially of 2 or 3 bacterial strains.

A composition, e.g., a pharmaceutical unit provided herein, may include each bacterial strain at any appropriate ratio, measured either by total mass or by colony forming units of the bacteria. For example, a disclosed pharmaceutical composition or unit may include two strains at a ratio of 0.1:1, 0.2:1, 0.25:1, 0.5:1, 0.75:1, 1:1, 2:1, 3:1, 4:1, 5:1, or 10:1, either by total mass or by colony forming units of the bacteria. For example, a disclosed pharmaceutical composition or unit may include three strains at a ratio of 1:1:1, 1:1:2, 1:1:4, 1:2:1, 1:2:2, 1:2:4, 1:4:1, 1:4:2, 1:4:4, 2:1:1, 2:1:2, 2:1:4, 2:2:1, 2:4:1, 4:1:1, 4:1:2, 4:1:4, 4:2:1, 4:4:1, either by total mass or by colony forming units of the bacteria.

In certain embodiments, the composition comprises a bacterial strain of Butyricimonas faecihominis, and optionally, one or more additional strains or species of bacteria, wherein the composition: (i) increases production of one or more pro-inflammatory gene products, for example, IFN-γ, IL-1β, IL-6, IL-12p40, IL-12p70, IL-23, IL-27, TNF, and/or TRAIL, in a human cell, e.g., a THP-1 macrophage or monocyte or a PBMC.

Excipients

A bacterial strain of Butyricimonas disclosed herein may be combined with pharmaceutically acceptable excipients to form a pharmaceutical composition, which can be administered to a patient by any means known in the art. As used herein, the term “pharmaceutically acceptable excipient” is understood to mean one or more of a buffer, carrier, or excipient suitable for administration to a subject, for example, a human subject, without excessive toxicity, irritation, allergic response, or other problem or complication, commensurate with a reasonable benefit/risk ratio. The excipient (s) should be “acceptable” in the sense of being compatible with the other ingredients of the formulations and not deleterious to the recipient.

Pharmaceutically acceptable excipients include buffers, solvents, dispersion media, coatings, isotonic and absorption delaying agents, and the like, that are compatible with pharmaceutical administration. Pharmaceutically acceptable excipients also include fillers, binders, disintegrants, glidants, lubricants, and any combination(s) thereof. For example, a contemplated composition may comprise a pharmaceutical excipient selected from the group consisting of cellulose, polyvinyl pyrrolidone, silicon dioxide, stearyl fumarate or a pharmaceutically acceptable salt thereof, lactose, starch, glucose, methyl cellulose, ethyl cellulose, hydroxypropyl methyl cellulose, magnesium stearate, mannitol, sorbitol, and any combination(s) thereof. For further examples of excipients, carriers, stabilizers and adjuvants, see, e.g., Handbook of Pharmaceutical Excipients, 8^{th} Ed., Edited by P. J. Sheskey, W. G. Cook, and C. G. Cable, Pharmaceutical Press, London, UK [2017]. The use of such media and agents for pharmaceutically active substances is known in the art.

Stabilized Bacterial Compositions

In certain embodiments, bacterial strains of Butyricimonas described herein may be used in any composition in stabilized form, including, for example, in a lyophilized state (with optionally one or more appropriate cryoprotectants), frozen (e.g., in a standard or super-cooled freezer), spray dried, and/or freeze dried. Stabilized bacteria (e.g. via lyophilization, freezing, spray drying or freeze drying), and in particular, stabilized anaerobic bacteria may, in certain embodiments, possess advantageous properties over bacteria in culture with respect to administration, e.g., administration of a pharmaceutical composition provided herein. For example, lyophilizing bacterial strains involves a freeze-drying process that removes water from the bacterial cells. The resulting lyophilized bacterial strains may, in certain embodiments, have enhanced stability as compared to bacterial cultures, and thus may be stored for longer periods of time (i.e. extending shelf-life). In addition, in certain embodiments, in stabilized form, dehydrated bacterial cells do not grow or reproduce, but remain viable and may grow and reproduce when rehydrated. In certain embodiments, viability of stabilized anaerobic Butyricimonas bacteria is maintained even when exposed to oxygen, thus facilitating their formulation (for example, into oral dosage forms) and use as a live biotherapeutic product that retains biological activity. Thus, in particular embodiments, the bacterial strains of Butyricimonas described herein are stabilized (e.g. via lyophilization, freezing, freeze-drying or spray-drying), live and viable, and retain some, most, or all of its chemical stability, and/or biological activity upon storage. Stability can be measured at a selected temperature and humidity conditions for a selected time period. Trend analysis can be used to estimate an expected shelf life before a material has actually been in storage for that time period. For live bacteria, for example, stability may be measured as the time it takes to lose 1 log of cfu/g dry formulation under predefined conditions of temperature and/or humidity. Alternatively, stability may be measured as the time required to measure a decrease in a particular biological function per unit of dry formulation.

In certain embodiments, a pharmaceutical composition or pharmaceutical unit comprising Butyricimonas faecihominis loses at most 0.5 log cfus, 1 log cfus, 1.5 log cfus, 2 log cfus, 2.5 log cfus, 3 log cfus, 3.5 log cfus, 4 log cfus, 4.5 log cfus, 5 log cfus, 5.5 log cfus, 6 log cfus, 6.5 log cfus, 7 log cfus, 7.5 log cfus, 8 log cfus, 8.5 log cfus, 9 log cfus, 9,5 log cfus, or 10 log cfus (total, or per gram of dry formulation) of each bacterial strain present in the pharmaceutical composition or unit upon storage for 1 month, 2 months, 3 months, 4 months, 5 months, 6 months, 7 months, 8 months, 9 months, 10 months 11, months, 12 months, 1 year, 1.5 years, 2 years, 2.5 years, 3 years, 3.5 years, 4 years, 4.5 years, or 5 years at 4° C. or −20° C. For example, the pharmaceutical composition or pharmaceutical unit may lose at most 3 log cfus of each bacterial strain present in the pharmaceutical composition or unit upon storage for 6 months, 1 year, or 2 years at 4° C.

A Butyricimonas bacteria disclosed herein may be combined with one or more cryoprotectants. Exemplary cryoprotectants include fructoligosaccharides (e.g., Raftilose®, fructooligosaccharide derived from inulin), trehalose, maltodextrin, sodium alginate, proline, glutamic acid, glycine (e.g., glycine betaine), mono-, di-, or polysaccharides (such as glucose, sucrose, maltose, lactose), polyols (such as mannitol, sorbitol, or glycerol), dextran, DMSO, methylcellulose, propylene glycol, polyvinylpyrrolidone, non-ionic surfactants such as Tween 80, and/or any combinations thereof.

In certain embodiments, the cryoprotectant comprises Raftilose® (fructooligosaccharide derived from inulin), maltodextrin, alginate, trehalose, and sucrose, or any combinations thereof. In some embodiments, a pharmaceutical composition comprising a bacterial strain of Butyricimonas further comprises sucrose as a cryoprotectant. In some embodiments, a pharmaceutical composition comprising a bacterial strain of Butyricimonas further comprises Raftilose® (fructooligosaccharide derived from inulin), maltodextrin, alginate, trehalose, and sucrose as cryoprotectants. In some embodiments, a pharmaceutical composition comprising a bacterial strain of Butyricimonas further comprises Raftilose® (fructooligosaccharide derived from inulin), maltodextrin, alginate, and trehalose as cryoprotectants.

In some embodiments, a lyophilized powder form of a bacterial strain, as contemplated herein, includes about 10% to about 80% (by weight) of one or more bacterial strains (e.g., one bacterial strain) and about 20% to about 90% (by weight) of one or more cryoprotectants and/or excipients, such as one or more cryoprotectants and/or excipients selected from the group consisting of Raftilose® (fructooligosaccharide derived from inulin), maltodextrin, sodium alginate, trehalose, sucrose, water, and/or combinations thereof. For example, 5 mg of contemplated lyophilized powder form of a bacterial strain may include about 0.5 mg to about 1.5 mg of the bacterial strain, about 1.5 mg to about 2.5 mg of the bacterial strain, about 2.5 to about 3.5 mg of the bacterial strain, or about 3.5 mg to about 4.5 mg of the bacterial strain. It can be appreciated that each lyophilized powder form of bacterial strain that may form a component of a disclosed composition may each have different excipients and/or amounts of excipients, as well as a discrete bacterial strain.

A pharmaceutical composition should be formulated to be compatible with its intended route of administration. The bacterial compositions disclosed herein can be prepared by any suitable method and can be formulated into a variety of forms and administered by a number of different means. The compositions can be administered orally, rectally, or enterally, in formulations containing conventionally acceptable carriers, adjuvants, and vehicles as desired. As used herein, “rectal administration” is understood to include administration by enema, suppository, or colonoscopy. A disclosed pharmaceutical composition may, e.g., be suitable for bolus administration or bolus release. In an exemplary embodiment, a disclosed bacterial composition is administered orally.

Solid dosage forms for oral administration include capsules, tablets, caplets, pills, troches, lozenges, powders, and granules. A capsule typically comprises a core material comprising a bacterial composition and a shell wall that encapsulates the core material. In some embodiments the core material comprises at least one of a solid, a liquid, and an emulsion. In some embodiments the shell wall material comprises at least one of a soft gelatin, a hard gelatin, and a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit®”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer functions as a taste-masking agent.

Tablets, pills, and the like can be compressed, multiply compressed, multiply layered, and/or coated. A contemplated coating can be single or multiple. In one embodiment, a contemplated coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments a contemplated coating material comprises a protein. In some embodiments a contemplated coating material comprises at least one of a fat and an oil. In some embodiments the at least one of a fat and an oil is high temperature melting. In some embodiments the at least one of a fat and an oil is hydrogenated or partially hydrogenated. In some embodiments the at least one of a fat and an oil is derived from a plant. In some embodiments the at least one of a fat and an oil comprises at least one of glycerides, free fatty acids, and fatty acid esters. In some embodiments a contemplated coating material comprises at least one edible wax. A contemplated edible wax can be derived from animals, insects, or plants. Non-limiting examples include beeswax, lanolin, bayberry wax, carnauba wax, and rice bran wax. Tablets and pills can additionally be prepared with enteric or reverse-enteric coatings.

Alternatively, powders or granules embodying a bacterial composition disclosed herein can be incorporated into a food product. In some embodiments a contemplated food product is a drink for oral administration. Non-limiting examples of a suitable drink include water, fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In certain embodiments, a pharmaceutical composition provided herein includes: (a) a Butyricimonas strain; and (b) a filler (e.g., microcrystalline cellulose, lactose, sucrose, mannitol, or dicalcium phosphate dihydrate), a disintegrant (e.g., polyvinyl pyrrolidone, sodium starch glycolate, starch, or carboxymethyl-cellulose), a flow-aid/glidant (e.g., talc or silica derivatives (e.g., colloidal silica such as Cab-O-Sil or Aerosil)), and a lubricant (e.g., sodium stearyl fumarate, magnesium stearate, calcium stearate, stearic acid, stearic acid salt, talc, liquid paraffin, propylene glycol (PG), PEG 6000, or magnesium/sodium lauryl sulfate).

In certain embodiments, a contemplated pharmaceutical composition includes: (a) a Butyricimonas strain; and (b) a filler (microcrystalline cellulose), a disintegrant (polyvinyl pyrrolidone), a flow-aid/glidant (silicon dioxide), and a lubricant (sodium stearyl fumarate).

In certain embodiments, a contemplated pharmaceutical composition is formulated as a capsule. In certain embodiments, the capsule is a hydroxypropyl methylcellulose (HPMC) capsule. In certain embodiments, the capsule includes a banding polymer (e.g., hydroxypropyl methylcellulose (HPMC)), and a banding solvent (e.g., water or ethanol). In certain embodiments, the capsule includes two banding solvents, water and ethanol. In certain embodiments the capsule is coated with a reverse enteric coating polymer (e.g., amino methacrylate copolymer), and comprises a surfactant (e.g., sodium lauryl sulfate), a flow-aid/glidant (e.g., silicon dioxide), a lubricant (e.g., stearic acid), an anti-tacking agent (e.g., talc), and a coating solvent (e.g., water). In certain embodiments the capsule is coated with an enteric coating polymer (e.g., poly (methacrylic acid-co-methyl methacrylate)), and further includes a plasticizer (e.g., triethyl citrate), an anti-tacking agent (e.g., talc), a pH adjuster (e.g., ammonia solution), and a coating solvent (e.g., purified water and isopropyl alcohol).

In certain embodiments, a contemplated capsule is a capsule-in-capsule dosage form, which includes an inner capsule and an outer capsule. In certain embodiments, the inner capsule includes one or more lyophilized bacterial strains, a filler (e.g., microcrystalline cellulose, lactose, sucrose, mannitol, dicalcium phosphate dihydrate, or starch), a disintegrant (e.g., polyvinyl pyrrolidone, sodium starch glycolate, or carboxymethyl-cellulose), a flow-aid/glidant (e.g., silicon dioxide, talc, or colloidal silica), and a lubricant (e.g., sodium stearyl fumarate, magnesium stearate, calcium stearate, stearic acid, stearic acid salt, talc, liquid paraffin, propylene glycol (PG), PEG 6000, or magnesium/sodium lauryl sulfate). In certain embodiments, the outer capsule includes one or more lyophilized bacterial strains, a filler (e.g., microcrystalline cellulose, lactose, sucrose, mannitol, dicalcium phosphate dihydrate, or starch). a disintegrant (e.g., polyvinyl pyrrolidone, sodium starch glycolate, or carboxymethyl-cellulose), a flow-aid/glidant (e.g., silicon dioxide, talc, or colloidal silica), and a lubricant (e.g., sodium stearyl fumarate, magnesium stearate, calcium stearate, stearic acid, stearic acid salt, talc liquid paraffin, propylene glycol (PG), PEG 6000, or magnesium/sodium lauryl sulfate).

In certain embodiments, a contemplated capsule is a capsule-in-capsule dosage form, which includes an inner capsule and an outer capsule. In certain embodiments, the inner capsule includes one or more lyophilized bacterial strains, a filler (microcrystalline cellulose), a disintegrant (polyvinyl pyrrolidone), a flow-aid/glidant (silicon dioxide), and a lubricant (sodium stearyl fumarate). In certain embodiments, the outer capsule includes one or more lyophilized bacterial strains, a filler (microcrystalline cellulose), a disintegrant (polyvinyl pyrrolidone), a flow-aid/glidant (silicon dioxide), and a lubricant (sodium stearyl fumarate).

In certain embodiments, a disclosed pharmaceutical unit comprises a dual component capsule. For example, a dual component capsule may comprise an inner capsule, wherein the inner capsule has a reverse enteric polymeric coating, and an outer capsule encapsulating the inner capsule, wherein the outer capsule has an enteric polymeric coating. A contemplated inner and/or outer capsule may comprise a bacterial strain or a bacterial strain mixture. For example, a dual component capsule may comprise an inner capsule having an inner composition comprising a bacterial strain or bacterial strain mixture and one or more pharmaceutical excipients, wherein the inner capsule has a reverse enteric polymeric coating, and an outer capsule encapsulating the inner capsule and an outer composition comprising a bacterial strain or bacterial strain mixture and one or more pharmaceutical excipients, wherein the outer capsule has an enteric polymeric coating. A contemplated inner and/or outer composition may, e.g., comprise a Butyricimonas strain, and optionally one or more additional strains. The inner composition and the outer composition may be the same or different.

A contemplated dual component capsule may include a total of about 5 mg to about 60 mg of the inner and outer composition, e.g., a total of about 5 mg to about 50 mg of the inner and outer composition, a total of about 5 mg to about 15 mg of the inner and outer composition, a total of about 5 mg to about 25 mg of the inner and outer composition, or a total of about 25 mg to about 50 mg of the inner and outer composition. A contemplated dual component capsule may include a total of about 50 mg to about 120 mg of the inner and outer composition, e.g., a total of about 50 mg to about 75 mg of the inner and outer composition, a total of about 60 mg to about 85 mg of the inner and outer composition, a total of about 50 mg to about 95 mg of the inner and outer composition, or a total of about 25 mg to about 110 mg of the inner and outer composition.

In certain embodiments, a disclosed dual component capsule includes an inner capsule with a reverse enteric polymeric coating, and an outer capsule with an enteric polymeric coating. Each respective coating, for example, allows for biphasic release of the capsule's contents (including bacterial strains) at distinct sites along the gastrointestinal tract. For example, it has been determined that the GI tract has several regions sharply demarcated by local pH ranging from 1 to 8.2. The normal pH profile of the GI tract rises and falls between the stomach and the colon with pH ranges of 1-4 in the stomach, 5.5-6.4 in the duodenum, 6.8-8.2 in the ileum, and 5.5-6.5 in the colon. For example, while the distal ileum contains a region where the usual pH is between 6.8 and 8.2, the pH drops sharply from 8.2 to 5.5 after passage through the ileocecal valve into the cecum and ascending colon. The pH gradually rises once again to 8.0 in the progression from proximal to distal colon. Accordingly, in certain embodiments, the enteric polymeric coating of the outer capsule solubilizes in a pH of about 7 to 8, allowing for release in the ileum, and the reverse enteric polymeric coating of the inner capsule solubilizes in a pH of about 6.2 to 6.5, allowing for subsequent release in the colon. In certain embodiments, the outer capsule maintains integrity (e.g., absence of splits, cracks, or rupture of capsule shell) for about 2 hours at pH 1.2 and 37° C. In certain embodiments, the outer capsule maintains integrity (e.g., absence of splits, cracks, or rupture of capsule shell) for about 2 hours at pH 5.5 and 37° C. In certain embodiments, the outer capsule disintegrates within about 1 hour at pH 7.4 and 37° C. In certain embodiments, the inner capsule maintains integrity (e.g., absence of splits, cracks, or rupture of capsule shell) for up to 1 hour at pH 7.4 and 37° C. In certain embodiments, the inner capsule disintegrates within 2 hours at pH 6.5 and 37° C.

In certain embodiments, the inner and/or outer capsule coating is comprised of poly(dl-lactide-co-glycolide, chitosan (Chi) stabilized with PVA (poly-vinylic alcohol), a lipid, an alginate, carboxymethylethylcellulose (CMEC), cellulose acetate trimellitiate (CAT), hydroxypropylmethyl cellulose phthalate (HPMCP), hydroxypropylmethyl cellulose, ethyl cellulose, food glaze, mixtures of hydroxypropylmethyl cellulose and ethyl cellulose, polyvinyl acetate phthalate (PVAP), cellulose acetate phthalate (CAP), shellac, copolymers of methacrylic acid and ethyl acrylate, or copolymers of methacrylic acid and ethyl acrylate to which a monomer of methylacrylate has been added during polymerization. Methylmethacrylates or copolymers of methacrylic acid and methylmethacrylate are available as Eudragit® polymers (Evonik Industries, Darmstadt, Germany). For example, Eudragit® L100 and Eudragit® S100 (anionic copolymers based on methacrylic acid and methyl methacrylate) can be used, either alone or in combination. Eudragit® L100 dissolves at about pH 6 and upwards and comprises between 46.0% and 50.6% methacrylic acid units per g dry substance; Eudragit® S100 dissolves at about pH 7 and upwards and comprises between 27.6% and 30.7% methacrylic acid units per g dry substance. Another exemplary group of encapsulating polymers are the polyacrylic acids Eudragit® L and Eudragit® S which optionally may be combined with Eudragit® RL or RS (copolymers of ethyl acrylate, methyl methacrylate and a low content of methacrylic acid ester with quaternary ammonium groups). These modified acrylic acids are useful since they can be made soluble at a pH of 6 to 7.5, depending on the particular Eudragit chosen, and on the proportion of Eudragit® S to Eudragit® L, RS, and RL used in the formulation. In certain embodiments, a contemplated coating of the inner capsule is comprised of Eudragit EPO® ReadyMix. In certain embodiments, a contemplated coating of the outer capsule is comprised of Eudragit® L100 (methylacrylic acid-methyl methacrylate co-polymer (1:1)) and Eudragit® 5100 (methylacrylic acid-methyl methacrylate co-polymer (1:2)). In certain embodiments, a contemplated capsule is suitable for extended or timed release. In certain embodiments, a contemplated inner and/or outer capsule coating further comprises a band/seal, e.g., hypromellose, an opacifier, e.g., titanium dioxide, a plasticizer, e.g. triethyl citrate (TEC) or an anti-tacking agent, e.g. talc.

Further exemplary capsule-in-capsule formulations are described in U.S. Pat. No. 9,907,755.

Unit Dosage Forms

Pharmaceutical compositions comprising a Butyricimonas strain disclosed herein can be presented in a unit dosage form, i.e., a pharmaceutical unit. A composition, e.g., a pharmaceutical unit provided herein, may include any appropriate amount of one or more bacterial strains, measured either by total mass or by colony forming units of the bacteria.

For example, a disclosed pharmaceutical composition or unit may include from about 10³ cfus to about 10¹² cfus, about 10⁶ cfus to about 10¹² cfus, about 10⁷ cfus to about 10¹² cfus, about 10⁸ cfus to about 10¹² cfus, about 10⁹ cfus to about 10¹² cfus, about 10¹⁰ cfus to about 10¹² cfus, about 10¹¹ cfus to about 10¹² cfus, about 10³ cfus to about 10¹¹ cfus, about 10⁶ cfus to about 10¹¹ cfus, about 10⁷ cfus to about 10¹¹ cfus, about 10⁸ cfus to about 10¹¹ cfus, about 10⁹ cfus to about 10¹¹ cfus, about 10¹⁰ cfus to about 10¹¹ cfus, about 10³ cfus to about 10¹⁰ cfus, about 10⁶ cfus to about 10¹⁰ cfus, about 10⁷ cfus to about 10¹⁰ cfus, about 10⁸ cfus to about 10¹⁰ cfus, about 10⁹ cfus to about 10¹⁰ cfus, about 10³ cfus to about 10⁹ cfus, about 10⁶ cfus to about 10⁹ cfus, about 10⁷ cfus to about 10⁹ cfus, about 10⁸ cfus to about 10⁹ cfus, about 10³ cfus to about 10⁸ cfus, about 10⁶ cfus to about 10⁸ cfus, about 10⁷ cfus to about 10⁸ cfus, about 10³ cfus to about 10⁷ cfus, about 10⁶ cfus to about 10⁷ cfus, or about 10³ cfus to about 10⁶ cfus of each bacterial strain, or may include about 10³ cfus, about 10⁶ cfus, about 10⁷ cfus, about 10⁸ cfus, about 10⁹ cfus, about 10¹⁰ cfus, about 10¹¹ cfus, or about 10¹² cfus of a bacterial strain or of each bacterial strain in the composition.

For example, a disclosed pharmaceutical composition or unit may include from about 10³ cfus to about 10¹² cfus, about 10⁶ cfus to about 10¹² cfus, about 10⁷ cfus to about 10¹² cfus, about 10⁸ cfus to about 10¹² cfus, about 10⁹ cfus to about 10¹² cfus, about 10¹⁰ cfus to about 10¹² cfus, about 10¹¹ cfus to about 10¹² cfus, about 10³ cfus to about 10¹¹ cfus, about 10⁶ cfus to about 10¹¹ cfus, about 10⁷ cfus to about 10¹¹ cfus, about 10⁸ cfus to about 10¹¹ cfus, about 10⁹ cfus to about 10¹¹ cfus, about 10¹⁰ cfus to about 10¹¹ cfus, about 10³ cfus to about 10¹⁰ cfus, about 10⁶ cfus to about 10¹⁰ cfus, about 10⁷ cfus to about 10¹⁰ cfus, about 10⁸ cfus to about 10¹⁰ cfus, about 10⁹ cfus to about 10¹⁰ cfus, about 10³ cfus to about 10⁹ cfus, about 10⁶ cfus to about 10⁹ cfus, about 10⁷ cfus to about 10⁹ cfus, about 10⁸ cfus to about 10⁹ cfus, about 10³ cfus to about 10⁸ cfus, about 10⁶ cfus to about 10⁸ cfus, about 10⁷ cfus to about 10⁸ cfus, about 10³ cfus to about 10⁷ cfus, about 10⁶ cfus to about 10⁷ cfus, or about 10³ cfus to about 10⁶ cfus of each bacterial strain, or may include about 10³ cfus, about 10⁶ cfus, about 10⁷ cfus, about 10⁸ cfus, about 10⁹ cfus, about 10¹⁰ cfus, about 10¹¹ cfus, or about 10¹² cfus of a bacterial strain in the composition.

In certain embodiments, a provided pharmaceutical unit comprises at least 1×10³ colony forming units of each bacterial strain (e.g., vegetative bacterial strain), or, at least 1×10⁴ colony forming units of bacterial strain (e.g., vegetative bacterial strain), or, at least 1×10⁵ colony forming units of bacterial strain (e.g., vegetative bacterial strain), or, at least 1×10⁶ colony forming units of each bacterial strain (e.g., vegetative bacterial strain), or, at least 1×10⁷ colony forming units of each bacterial strain (e.g., vegetative bacterial strain), or, at least 1×10⁸ colony forming units of each bacterial strain (e.g., vegetative bacterial strain), or, at least 1×10⁹ colony forming units of each bacterial strain (e.g., vegetative bacterial strain).

For example, disclosed compositions (e.g., a pharmaceutical unit such as e.g., a capsule) can include about 1 mg to about 5 mg (e.g., 2 mg to about 4 mg) of a bacterial strain, which can each be present in the unit, e.g., within about 5 mg to about 50 mg of a lyophilized powder form of the bacterial strain. For example, a pharmaceutical unit may comprise a total of about 30 mg to about 70 mg, about 30 mg to about 60 mg, about 30 mg to about 50 mg, about 30 mg to about 40 mg, about 40 mg to about 70 mg, about 40 mg to about 60 mg, about 40 mg to about 50 mg, about 50 mg to about 70 mg, about 50 mg to about 60 mg, about 80 mg to about 100 mg, about 90 mg to about 110 mg, about 100 mg to about 120 mg, or about 110 mg to about 150 mg of lyophilized powder forms of the bacterial strain. In certain embodiments, the pharmaceutical unit comprises a total of about 30 mg, 40 mg, 50 mg, 60 mg, 70 mg, 100 mg, 120 mg, 130 mg, 140 mg, or 150 mg of lyophilized powder form of the bacterial strain.

In certain embodiments, a disclosed composition such as a disclosed pharmaceutical unit may include about 5 to about 50 mg of each lyophilized powder form of a bacterial strain, for example, about 5 to about 45 mg, about 5 to about 40 mg, about 5 to about 35 mg, about 5 to about 30 mg, about 5 to about 25 mg, about 5 to about 15 mg, about 5 to about 10 mg, about 10 to about 50 mg, about 10 to about 35 mg of each lyophilized powder form of a bacterial strain (e.g., a vegetative bacterial strain), about 10 to about 20 mg, about 10 to about 15 mg, or about 15 to about 45 mg of each lyophilized powder form of a bacterial strain (e.g., a vegetative bacterial strain). In certain embodiments, a disclosed pharmaceutical unit comprises about 5, about 10, about 15, about 20, about 25, or about 30 mg of each lyophilized powder form of a bacterial strain (e.g., a vegetative bacterial strain). In certain embodiments, a disclosed pharmaceutical unit includes about 25 to about 50 mg of a lyophilized powder form of one bacterial strain (e.g., vegetative bacterial strain) and about 5 mg to about 10 mg of the remaining lyophilized powder forms of bacterial strains (e.g., vegetative bacterial strains), or about 5 to about 15 mg of one lyophilized powder form of bacterial strain (e.g., vegetative bacterial strain) and about 5 to 10 mg of the remaining lyophilized powder forms of bacterial strains (e.g., vegetative bacterial strains), for example, about 15 mg of one lyophilized powder form of bacterial strain (e.g., vegetative bacterial strain) and about 5 mg of the remaining lyophilized powder forms of bacterial strains (e.g., vegetative bacterial strains), or about 15 mg to about 25 mg of each of two lyophilized powder forms of bacterial strains (e.g., vegetative bacterial strains) and about 5 mg to 10 mg of the remaining lyophilized powder forms of bacterial strains (e.g., vegetative bacterial strains).

In certain embodiments a pharmaceutical composition or unit may include, or may be administered in combination with a prebiotic, i.e., a compound or composition which modifies the growth, maintenance, activity and/or balance of the intestinal micro flora (e.g., can allow for specific changes in the composition and/or activity of the microbiome). Exemplary prebiotics include complex carbohydrates, complex sugars, resistant dextrins, resistant starch, amino acids, peptides, nutritional compounds, biotin, polydextrose, fructooligosaccharide (FOS), galactooligosaccharides (GOS), inulin, lignin, psyllium, chitin, chitosan, chitosanoligosaccharides, lacitol, gums (e.g., guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, isomaltoligosaccharides, and xylooligosaccharides (XOS). Prebiotics can be found in foods (e.g., acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, and banana), and breast milk. Prebiotics can also be administered in other forms (e.g., a capsule or dietary supplement).

### III. Therapeutic Uses

Compositions and methods disclosed herein can be used to treat various forms of inflammatory disorders, gastrointestinal disorders, and/or dysbiosis in a subject. The disclosure provides a method of treating a gastrointestinal disorder, inflammatory disorder, and/or dysbiosis in a subject. A contemplated method comprises administering to the subject an effective amount of a pharmaceutical composition and/or pharmaceutical unit comprising a Butyricimonas bacterial strain disclosed herein (and optionally one or more additional bacterial strains), either alone or in a combination with another therapeutic agent to treat the gastrointestinal disorder, inflammatory disorder, and/or dysbiosis in the subject.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals, e.g., human, a companion animal (e.g., dog, cat, or rabbit), or a livestock animal (for example, cow, sheep, pig, goat, horse, donkey, and mule, buffalo, oxen, or camel)).

It will be appreciated that the exact dosage of a pharmaceutical unit, pharmaceutical composition, or bacterial strain is chosen by an individual physician in view of the patient to be treated, in general, dosage and administration are adjusted to provide an effective amount of the bacterial agent to the patient being treated. As used herein, the “effective amount” refers to the amount necessary to elicit a beneficial or desired biological response. An effective amount can be administered in one or more administrations, applications or dosages and is not intended to be limited to a particular formulation or administration route. As will be appreciated by those of ordinary skill in this art, the effective amount of a pharmaceutical unit, pharmaceutical composition, or bacterial strain may vary depending on such factors as the desired biological endpoint, the drug to be delivered, the target tissue, the route of administration, etc. Additional factors which may be taken into account include the severity of the disease state; age, weight and gender of the patient being treated; diet, time and frequency of administration; drug combinations; reaction sensitivities; and tolerance/response to therapy.

It is understood that a disclosed bacterial strain, bacterial strain mixture, or composition may not require colonization of the gut, e.g., an intestine, of the subject and/or persistence in the subject in order elicit a beneficial or desired biological response. For example, in certain embodiments, a bacterial strain, bacterial strain mixture, or composition colonizes or partially colonizes the gut of the subject and/or persists in the subject after administration. In certain embodiments, a bacterial strain, bacterial strain mixture, or composition does not colonize the gut of the subject and/or persist in the subject after administration.

Compositions and methods disclosed herein can also be used to treat cancer in a subject. A contemplated method comprises administering to the subject an effective amount of a pharmaceutical composition and/or pharmaceutical unit comprising a Butyricimonas bacterial strain disclosed herein (and optionally one or more additional bacterial strains), either alone or in a combination with another therapeutic agent to treat the cancer in the subject. Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma). In certain embodiments, the cancer is colorectal cancer (CRC).

In other embodiments, the compositions and methods disclosed herein may also be useful for preventing one or more of the above diseases or conditions, when administered as vaccine compositions. In certain such embodiments, the bacterial strains provided herein are viable. In certain such embodiments, the bacterial strains are capable of at least partially or totally colonizing the gastrointestinal tract, e.g., the intestine. In certain such embodiments, the bacterial strains of the invention are viable and capable of at least partially or totally colonizing the gastrointestinal tract, e.g., the intestine. In other certain such embodiments, the bacterial strains of the invention may be killed, inactivated or attenuated. In certain such embodiments, the compositions may comprise a vaccine adjuvant. In certain embodiments, the compositions are for administration via injection, such as via subcutaneous injection.

### IV. Combination Therapy

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. In certain embodiments, a side effect of a first and/or second treatment is reduced because of combined administration.

In certain embodiments, a pharmaceutical composition or unit may include, or be administered in combination with, chemotherapy, e.g., a cytotoxic agent. Exemplary cytotoxic agents include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In certain embodiments, the cytotoxic agent is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

In certain embodiments, a pharmaceutical composition or unit may include, or be administered in combination with, a targeted therapy, e.g. a tyrosine kinase inhibitor, a proteasome inhibitor, or a protease inhibitor. In certain embodiments, a pharmaceutical composition or unit may include, or be administered in combination with, an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, a pharmaceutical composition or unit may be administered in combination with surgery or radiation therapy.

In certain embodiments, a pharmaceutical composition or unit may include, or be administered in combination with, a checkpoint inhibitor. The checkpoint inhibitor may, for example, be selected from a PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include nivolumab (OPDIVO®, Bristol-Myers Squibb), pembrolizumab (KEYTRUDA®, Merck), cemiplimab (LIBTAYO®, Regeneron/Sanofi), spartalizumab (PDR001), MEDI0680 (AMP-514), and pidilizumab (CT-011). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include avelumab (BAVENCIO®, EMD Serono/Pfizer), atezolizumab (TECENTRIQ®, Genentech), durvalumab (IMFINZI®, Medimmune/AstraZeneca), and BMS 936559 (Bristol Myers Squibb Co.).

In certain embodiments, the checkpoint inhibitor is a CTLA-4 inhibitor. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, a pharmaceutical composition or unit may include, or be administered in combination with, an anti-bacterial agent, e.g., an antibiotic. A disclosed method may comprise pretreatment with an antibiotic, e.g., administration of an antibiotic to a subject prior to administration of a disclosed pharmaceutical composition or unit. Exemplary antibiotics for use in combination therapy include vancomycin, metronidazole, gentamicin, colistin, fidaxomicin, telavancin, oritavancin, dalbavancin, daptomycin, cephalexin, cefuroxime, cefadroxil, cefazolin, cephalothin, cefaclor, cefamandole, cefoxitin, cefprozil, ceftobiprole, cipro, Levaquin, floxin, tequin, avelox, norflox, tetracycline, minocycline, oxytetracycline, doxycycline, amoxicillin, ampicillin, penicillin V, dicloxacillin, carbenicillin, methicillin, ertapenem, doripenem, imipenem/cilastatin, meropenem, amikacin, kanamycin, neomycin, netilmicin, tobramycin, paromomycin, cefixime, cefdinir, cefditoren, cefoperazone, cefotaxime, ceftazidime, ceftibuten, ceftizoxime, ceftriaxone, cefoxotin, and/or streptomycin.

In certain embodiments, a pharmaceutical composition or unit may include, or be administered in combination with, an anti-fungal or anti-viral agents. Exemplary anti-viral agents include abacavir, acyclovir, adefovir, amprenavir, atazanavir, cidofovir, darunavir, delavirdine, didanosine, docosanol, efavirenz, elvitegravir, emtricitabine, enfuvirtide, etravirine, famciclovir, foscarnet, fomivirsen, ganciclovir, indinavir, idoxuridine, lamivudine, lopinavir, maraviroc, MK-2048, nelfinavir, nevirapine, penciclovir, raltegravir, rilpivirine, ritonavir, saquinavir, stavudine, tenofovir trifluridine, valaciclovir, valganciclovir, vidarabine, ibacitabine, amantadine, oseltamivir, rimantidine, tipranavir, zalcitabine, zanamivir and zidovudine. Exemplary anti-fungal agents include natamycin, rimocidin, filipin, nystatin, amphotericin B, candicin, and hamycin, miconazole, ketoconazole, clotrimazole, econazole, omoconazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, terconazole, and albaconazole, abafungin, terbinafine, naftifine, butenafine, anidulafungin, caspofungin, micafungin, polygodial, benzoic acid, ciclopirox, tolnaftate, undecylenic acid, flucytosine or 5-fluorocytosine, griseofulvin, and haloprogin.

In certain embodiments, a pharmaceutical composition or unit may include, or be administered in combination with, at least one or more additional strains or species of bacteria. In some embodiments, the at least one additional strain or species of bacteria in the composition is a bacterial strain of the genus Butyricimonas. For example, a pharmaceutical composition or unit may include, or be administered in combination with, an additional strain of Butyricimonas faecihominis and/or one or more strains of a Butyricimonas species that is not Butyricimonas faecihominis. Exemplary additional Butyricimonas species include Butyricimonas synergistica, Butyricimonas faecalis, Butyricimonas virosa and Butyricimonas paravirosa. In other embodiments, a pharmaceutical composition or unit may include, or be administered in combination with, one or more non-Butyricimonas bacterial species. In some embodiments, the one or more non-Butyricimonas bacterial species includes Collinsella ASMB P121-D5a. In some embodiments, the one or more non-Butyricimonas bacterial species includes an Alistipes species, for example, Alistipes senegalensis. In a particular embodiment, the Alistipes senegalensis is Alistipes senegalensis strain P150-D12a. In some embodiments, the one or more non-Butyricimonas bacterial species includes (i) a member of the genus Collinsella, for example, Collinsella ASMB P121-D5a, and (ii) a member of the genus Alistipes, for example, Alistipes senegalensis, for example, Alistipes senegalensis strain P150-D12a. In yet other embodiments, a pharmaceutical composition or unit may include, or be administered in combination with, a Butyricimonas strain selected from Butyricimonas synergistica, Butyricimonas faecalis, Butyricimonas virosa and Butyricimonas paravirosa, and one or more non-Butyricimonas bacterial species.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure. For example, it will be appreciated that all features described and depicted herein can be applicable to all aspects of the disclosure described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present disclosure remains operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

### EXAMPLES

The following Examples are merely illustrative and are not intended to limit the scope or content of the disclosure in any way.

### Example 1—Isolation and Purification of Butyricimonas faecihominis

1.1 Source. Isolate P40-F2a was isolated from the stool sample of a healthy human donor. The donor underwent comprehensive clinical and laboratory testing to confirm healthy status including screening for infectious agents to minimize risk of transmissible infection. Serology screening included HIV-1/HIV-2 (IgG and EIA), HTLV-I and HTLV-II (Ab), Hepatitis A virus (IgM), Hepatitis B virus (HBSAg, anti-HBc IgG and IgM), Hepatitis C virus (anti-HCV IgG), Treponema pallidum (EIA, or RPR if EIA is positive), Strongyloides stercoralis Ab, CMV Viral Load, and EBV Viral Load. Stool screening included Clostridium difficile toxin A/B (PCR), routine bacterial culture for enteric pathogens (with enrichment) including H. pylori EIA, Salmonella, Shigella, Yersinia, Campylobacter, and Vibrio, E. coli 0157 (perform E. coli 0157 culture, if stx1/2 EIA +ve), Shiga-like toxins stx1/2 (Shigella) EIA, Culture-based assays for vancomycin-resistant Enterococcus (VRE), extended spectrum beta-lactamase (ESBL) producers, carbapenem-resistant Enterobacteriaceae (CRE), and methicillin-resistant Staphylococcus aureus (MRSA), Giardia antigen (EIA), Cryptosporidium antigen (EIA), Cyclospora, Isospora, and Microsporidia (Microscopic observation with acid fast stain), Ova and Parasites (Microscopic observation), Rotavirus (EIA), Norovirus GI/GII (RT-PCR), and Adenovirus 40,41 EIA.

1.2 Isolation and Purification. Dilutions of donor samples were plated on isolation media at 37° C. Colonies were picked from isolation media agar plates (YCFAC, BHI supplemented with Vitamin K and Hemin) into a 96-well microtiter plate containing 200 μl of BHI+Hemin+Vitamin K. Once growth was observed visually in the 96-well microtiter plate, 20 μl of culture from each well of the 96-well microtiter plate was transferred into a 96-well Deep-Well plate containing 1 ml isolation media, followed by incubation at 37° C. After visually detecting growth, 1 ml of 50% glycerol was added to each well, and 600 μl of the mix was transferred into a Thermo Fisher Matrix tube plate. Individual cultures were subsequently plated on isolation media for conformation of colony morphology uniformity. Individual colonies were picked for identification by 16S sequencing and replated on YCFAC.

### Example 2—Taxonomic Characterization of Isolate P40-F2a

2.1 16S Sequencing and Phylogenetic Analysis.

A taxonomic characterization of purified isolate P40-F2a was performed using full length 16S rRNA gene sequencing data. Homology searches were performed against existing publicly available strains present in the National Center for Biotechnology Information (NCBI) taxonomy database.

2.1.1 16S rRNA gene sequencing. 50 μl of a liquid culture of isolate P40-F2a was denatured at 95° C. for 10 minutes. The denatured sample was utilized as a template to PCR amplify the 16S gene by using 16S rRNA primers 27F and 1492R. Sanger sequencing was performed (Elim Biopharm, Hayward, Calif.) using a set of 4 primers (27F, 1492R, 515F and 907R) to recover a near full length 16S rRNA gene fragment (SEQ ID NO: 846). The four amplicons were assembled into a single contiguous sequence using DNAbaser (Heracle BioSoft S. R. L., Arges, Romania) which was then searched against the NCBI database using BLASTn.

2.1.2 Phylogenetic analysis. A search with SEQ ID NO: 846 against the 16S rRNA gene database on NCBI yielded a closest match of 99.2% over 100% of the sequence length of 1415 bps (SEQ ID NO: 846). This match was from the species Butyricimonas faecihominis strain 180-3 (accession no. NR 126194.1). The next closest match over the entire length was of 97.39% identity from the species Butyricimonas paravirosa strain 214-4 (accession no. NR 126195.1).

A summary of the taxonomic identification results is provided in Table 1.

TABLE 1
Taxonomic Identification Result Summary

| | Isolate 16S sequence | Percent |
|---|---|---|
| Database match | coverage | Identity |
| Butyricimonas faecihominis strain 180-3 | 100% | 99.22% |
| Butyricimonas paravirosa strain 214-4 | 100% | 97.39% |
| Butyricimonas virosa strain MT12 | 100% | 96.79% |
| Butyricimonas synergistica strain MT01 | 100% | 94.07% |

2.2 Whole Genome Sequencing and Analysis.

2.2.1 Sequencing. DNA extraction, sequencing, quality filtering, assembly and annotation was performed by Corebiome, Inc. (Minneapolis, Minn.). DNA was extracted from isolate P40-F2a with MO Bio PowerFecal (Qiagen) automated for high throughput on QiaCube (Qiagen), with bead beating in 0.1 mm glass bead plates. Samples were quantified with Qiant-iT Picogreen dsDNA Assay (Invitrogen). Libraries were prepared with a proprietary procedure adapted for the Nextera Library Prep kit (Illumina) and sequenced on an Illumina NextSeq using single-end 1×150 reads with a NextSeq 500/550 High Output v2 kit (Illumina). DNA sequences were filtered for low quality (Q-Score <20) and length (<50), and adapter sequences were trimmed using cutadapt v.1.15 (Martin, EMBnet Journal, [Si], v. 17, n. 1, p. pp. 10-12, (2011)).

2.2.2 Assembly and Annotation. Sequences were assembled using SPAdes v3.11.0 (Bankevich et al., J Comput Biol. 19(5):455-477 (2012)). Protein annotation was performed with Prokka v 1.12 (Seemann, Bioinformatics 30(14):2068-2069 (2014)) on contigs over 1,000 bases in length.

2.2.3 Quality Assessment. Sequencing quality was determined by inspecting quality scores generated by FASTQC, with bases of low quality indicated by scores less than 20. Assembly quality metrics were generated by QUAST v.4.5 (Gurevich et al., Bioinformatics 29(8):1072-1075 (2013)).

2.2.4 Taxonomy. Taxonomic identities were made using appropriate score cut-offs on average nucleotide identity and alignment fraction scores.

2.2.5 Genome Characteristics. Intrinsic properties of the isolate P40-F2a genome assembly are summarized in Table 2 below.

TABLE 2
Characteristics of P40-F2a PI assemblies.

| | |
|---|---|
| Genome Size (Mb) | 4.85 |
| Contigs | 845 |
| G + C Content | 43.47% |
| # CDS | 3904 |
| # tRNA | 49 |

PI = primary isolate;
Mb = megabase pairs;
CDS = coding sequence,
tRNA = transfer ribonucleic acid

2.2.6 Genome wide similarity across P40-F2a and other members of Butyricimonas. The PI genome was compared against other strains of Butyricimonas faecihominis to measure the extent of genomic similarity, in particular, average nucleotide identity (ANI). The results, determined using FastANI (Jain et al., Nat Commun. 9(1):5114 (2018), are summarized in Table 3 below.

TABLE 3
Average Nucleotide Identity (ANI) of P40-F2a compared
to other strains of Butyricimonas faecihominis.

| Isolate | | ANI | ANI |
|---|---|---|---|
| ID (S) | Reference Species (R) | S → R | R → S |
| P40-F2a | Butyricimonas faecihominis strain CCUG | 99.89 | 99.51 |
| | 65562 (RefSeq assembly accession: | | |
| | GCF_008830385.1) | | |
| P40-F2a | Butyricimonas faecihominis strain 30A1 | 99.58 | 99.40 |
| | (RefSeq assembly accession: | | |
| | GCF_003851945.2) | | |

2.2.7 Physiological and Metabolic Characteristics of P40-F2a

P40-F2a cells are obligate anaerobes, non-motile, non-spore forming, and appear as short rods. P40-F2a was evaluated for its ability to utilize 190 different carbon sources using Phenotypic Microarrays (Biolog, Hayward, Calif.). The carbon sources utilized by P40-F2a include α-D-Glucose, D-Mannose, α-D-Lactose, Glycerol, D-Galactose, N-Acetyl-D-glucosamine, D,L-α-Glycerol-Phosphate, D-Glucosamine, N-Acetyl-D-Galactosamine, N-Acetyl-Neuraminic Acid, α-Keto-Valeric Acid, and Melibionic Acid.

### Example 3—In vitro Functional Activity of Butyricimonas faecihominis P40-F2a

This example describes studies of the activity of Butyricimonas faecihominis P40-F2a in in vitro human epithelial, macrophage, monocyte and dendritic cell models.

### Example 3.1—Preparation of Freshly Cultured Bacterial Strains for Cell Culture Assays

Freshly cultured bacteria from overnight cultures of the Butyricimonas faecihominis P40-F2a strain and a negative control strain previously confirmed to have anti-inflammatory properties were prepared in anaerobic conditions. Bacteria were centrifuged at 4300×g for four minutes. Bacteria were washed once with pre-reduced anaerobic PBS (Gibco). Working stock solutions were prepared by resuspending washed bacteria with anaerobic PBS to the total surface area of ˜1×10\hat{ }10 μm². Total surface area was calculated by determining the number of particles (bacterial cells) in solution, then multiplying the total number of particles by the Average surface area (μm²) of each particle, as measured by a particle counter (Beckman Coulter Counter). 10-fold serial dilutions were made using anaerobic PBS for specific assays.

### Example 3.2—Human Monocyte-Derived Dendritic Cell (moDC) In Vitro Cytokine Assay

Cryopreserved PBMC were thawed in a 37° C. water bath, diluted in warm RPMI 1640 supplemented with 10% heat-inactivated FBS and L-glutamine, and centrifuged (515×g; four minutes). Cells were resuspended in PBS buffer containing 0.5% bovine serum albumin (BSA) and 2 mM EDTA and CD14+ monocyte cells were isolated by selection using Miltenyi CD14 Microbeads according to manufacturer's directions. Isolated CD14+ monocytes were cultured in RPMI 1640 supplemented with 10% heat-inactivated FBS, L-glutamine, penicillin/streptomycin antibiotic, 50 ng/mL recombinant human IL-4 (R&D Systems), and 100 ng/mL recombinant human GM-CSF (Biolegend). Media was replenished on days 3 and 6. On day 7 after isolation, cells were diluted to 5×10⁵ cells/mL in RPMI 1640 containing L-glutamine (Corning) supplemented with 10% heat-inactivated FBS (Tissue Culture Biologicals) and 0.292 mg/mL L-glutamine (Corning). A 100 μL aliquot of the 5×10⁵ cells/mL cell suspension was added to each well within a flat-well 96 well plate and cultured for 24 hours at 37° C. and 5% CO₂ before addition of test articles.

Bacterial test articles (P40-F2a and negative control strains) were prepared to a total surface area of 1×10\hat{ }8 μm² and 1×10\hat{ }7 μm², respectively. The test articles, vehicle (PBS) control and moDCs were co-incubated for 3 hours in 37° C. and 5% CO₂. The plates were then centrifuged (515×g; four minutes), media removed, and replaced with RPMI 1640 supplemented with 10% heat-inactivated FBS, L-glutamine, and penicillin/streptomycin antibiotic. Culture plates were then incubated for an additional 15 h at 37° C. and 5% CO₂. The plates were centrifuged (515×g; four minutes), and supernatant was collected and analyzed by a custom U-plex multiplex kit from Meso Scale Discovery according to manufacturer's instructions. Results were averaged from 4 human donors with two experimental replicates from each donor.

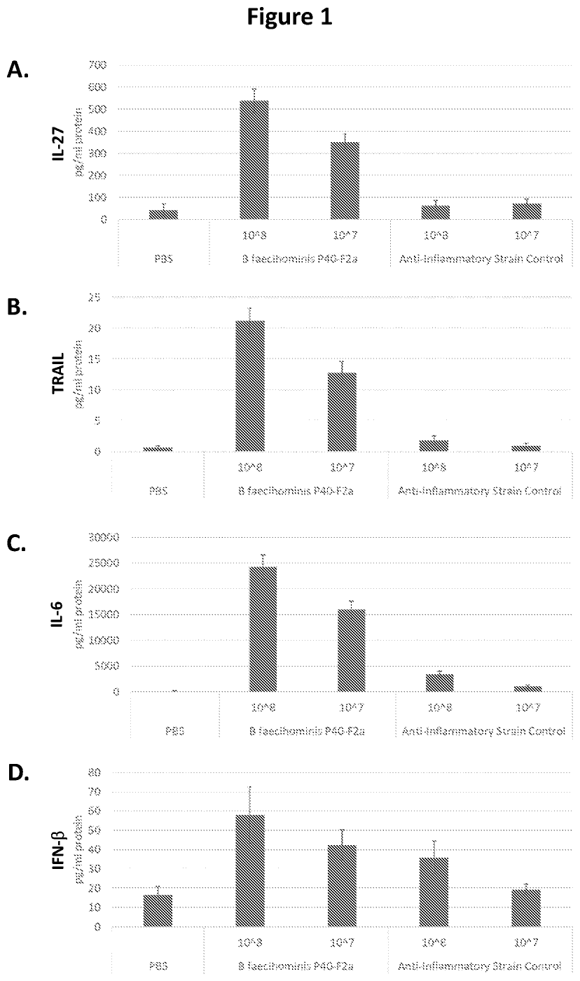

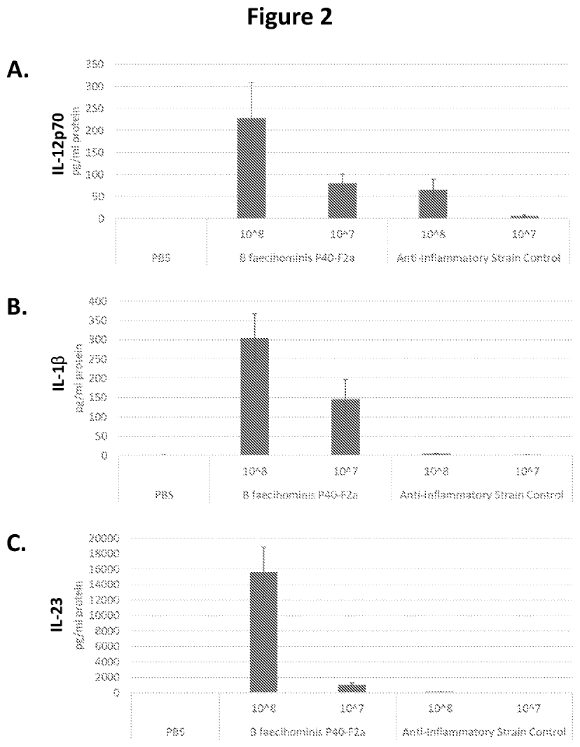

As shown in FIG. 1, Butyricimonas faecihominis P40-F2a induced significant, dose-dependent increases in the production of: (A) IL-27, (B) TRAIL, (C) IL-6 and (D) IFN-β by human monocyte-derived dendritic cells (moDCs), in comparison to production of these pro-inflammatory cytokines induced by the anti-inflammatory strain control and vehicle (PBS). As shown in FIG. 2, Butyricimonas faecihominis P40-F2a induced similar significant, dose-dependent increases in the production of: (A) IL-12p70, (B) IL-1β, and (C) IL-23, in human moDCs, in comparison to production of these pro-inflammatory cytokines induced by the anti-inflammatory strain control and vehicle (PBS).

### Example 3.3—Human PBMC In vitro Cytokine Assay

Trima residual blood product containing concentrated blood mononuclear cells was obtained from anonymous donors through Blood Centers of the Pacific (San Francisco, Calif.) and processed within 24 hours of collection. Blood samples were tested negative for HIV, HBV, HCV, HTLV, Syphilis, West Nile Virus and Zika Virus. PBMC were isolated using a ficoll gradient as described previously (Sim et al., J. Vis. Exp. (112), e54128 2016). Briefly, 50 mL of Trima residual was diluted with 50 mL of sterile PBS (Gibco) and 25 mL was overlaid on 15 mL Ficoll-Paque Plus (GE Healthcare) in 50 mL conical tubes. The samples were centrifuged at 450×g for 30 min at room temperature and allowed to stop without brake. The PBMC interphase was collected, washed with PBS and resuspended in RPMI 1640 containing 2.05 mM L-glutamine (Corning) supplemented with 10% heat-inactivated FBS (Tissue Culture Biologicals) and 0.292 mg/mL L-glutamine (Corning). The cells were maintained by incubation in 37° C. and 5% CO₂ and used for assay evaluation within 24 h or frozen for later use. Cells were cryopreserved in RPMI 1640 supplemented with 50% FBS and 10% DMSO (Sigma Aldrich) at a concentration of 5×10⁷ cells/mL and stored in liquid nitrogen until ready for use.

Human PBMCs, used immediately after isolation or thawed from cryo-storage, were diluted to 5×10⁶ cells/mL in RPMI 1640 containing L-glutamine (Corning) supplemented with 10% heat-inactivated FBS (Tissue Culture Biologicals) and 0.292 mg/mL L-glutamine (Corning). A 100 μL aliquot of the 5×10⁶ cells/mL cell suspension was added to each well within a round-bottom 96 well plate and cultured for 24 hours at 37° C. and 5% CO₂ before addition of test articles.

Test articles were prepared and added to the PBMCs as described above for the moDC assay. After 3 hours of incubation in 37° C. with 5% CO₂, the plates containing cocultures were centrifuged (515×g; four minutes), media removed, and replaced with RPMI 1640 supplemented with 10% heat-inactivated FBS, L-glutamine, and penicillin/streptomycin antibiotic. Culture plates were then incubated for an additional 15 h at 37° C. and 5% CO₂. The plates were centrifuged (515×g; four minutes) and supernatant was collected and analyzed by a custom U-plex multiplex kit from Meso Scale Discovery according to manufacturer's instructions. Results were averaged from 4 human donors with two experimental replicates from each donor.

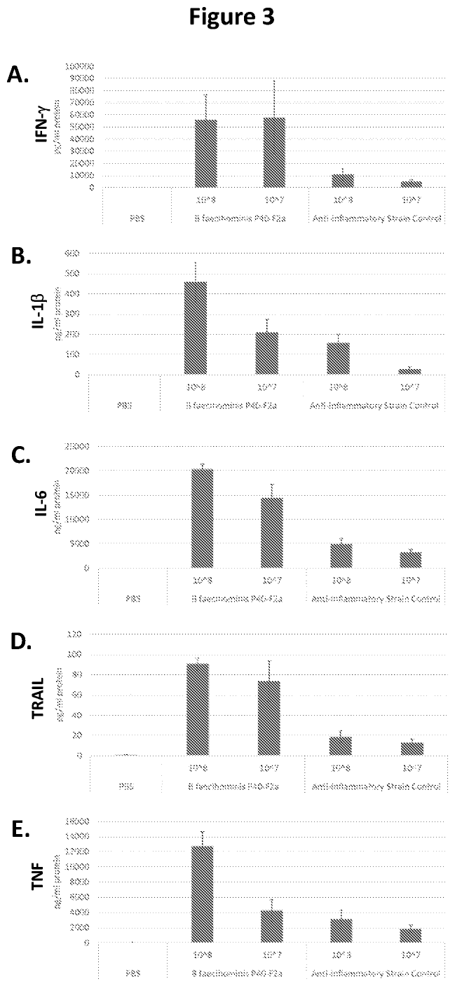

As shown in FIG. 3, Butyricimonas faecihominis P40-F2a induced significant, dose-dependent increases in the production of: (A) IFN-γ, (B) IL-1β, (C) IL-6, (D) TRAIL, and (E) TNF by human PBMCs, in comparison to production of these pro-inflammatory cytokines induced by the anti-inflammatory strain control and vehicle (PBS).

### Example 3.4—Human Macrophage and Monocyte In Vitro Cytokine and Chemokine Assay

The THP-1 human monocyte cell line (ATCC cat # TIB-202) was cultured in 37° C. and 5% CO₂ using RPMI 1640 containing 2.05 mM L-glutamine (Corning) supplemented with 10% heat-inactivated FBS (Corning), 100 I.U/mL Penicillin, 100 μg/mL Streptomycin and 0.292 mg/mL L-glutamine (Corning). Passage number was restricted to 8 passages. The THP-1 human monocyte cell line was grown until 70-80% confluent. Cells were counted and resuspended in culture media. 100,000 cells were plated per well onto 96 well plates. THP-1 human M2 macrophages were made by culturing the THP-1 human monocyte cells with 10 ng/mL phorbol 12-myristate 13-acetate (PMA) (InvivoGen) for 24 hours followed by 20 ng/mL IL-4 (R&D Systems) and 20 ng/mL IL-13 (R&D Systems) for 48 hours in 37° C. and 5% CO₂ as described previously (Genin et al., BMC Cancer 15:577 (2015)). One day before the experiment, cells were washed and resuspended in RPMI culture media without antibiotics containing 20 ng/mL IL-4 and 20 ng/mL IL-13.

Bacterial test articles (P40-F2a and negative control strains) were prepared to a total surface area of 1×10\hat{ }8 μm², 1×10\hat{ }7 μm², and 1×10\hat{ }6 μm² respectively. The test articles and vehicle (PBS) control were added onto THP-1 macrophages at 10% v/v and centrifuged down onto the THP-1 cells at 515×g for four minutes. The test articles, control and THP-1 macrophages were co-incubated for 3 hours in 37° C. and 5% CO₂. The co-incubation media was then replaced with fresh RPMI culture media supplemented with antibiotics to limit excess bacteria growth. THP-1 cells were incubated after culture media replacement for 15 hours in 37° C. and 5% CO₂. THP-1 cell supernatants were collected and analyzed by ELISA. Levels of selected cytokines in culture supernatants were quantified by using commercial enzyme-linked immunosorbent assay (ELISA) kits from Biolegend or R&D Systems with TMB detection according to manufacturer's specifications.

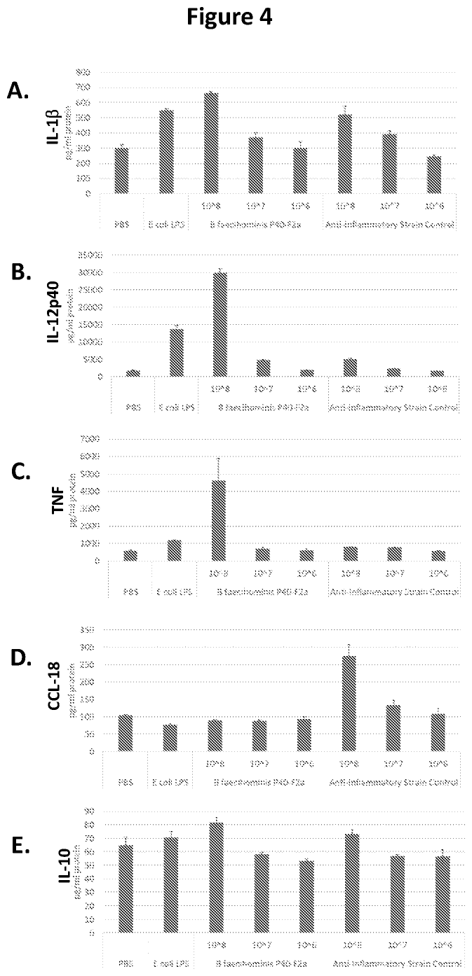

As shown in FIG. 4, Butyricimonas faecihominis P40-F2a induced significant, dose-dependent increases in the production of: (A) IL-1β, (B) IL-12p40, and (C) TNF by THP-1 M2 macrophages, in comparison to production of these pro-inflammatory cytokines induced by the anti-inflammatory strain control and vehicle (PBS). By contrast, Butyricimonas faecihominis P40-F2a did not significantly induce production of the anti-inflammatory cytokines (D) CCL-18 and (E) IL-10 in THP-1 M2 macrophages.

### Example 5—In Vivo Functional Activity of Butyricimonas faecihominis P40-F2a

Butyricimonas faecihominis P40-F2a was tested for efficacy in two different well-validated mouse tumor models: (1) a subcutaneous B16F10 syngeneic melanoma model and (2) a subcutaneous CT26 syngeneic colon carcinoma model.

5.1 B16F10 Syngeneic Melanoma Model

B16F10 cells were thawed from low passage number stock, expanded using standard tissue culture technique, and maintained in DMEM with 10% fetal bovine plasma (FBS) in a 37° C. incubator with 5% CO₂. Cells growing in logarithmic phase (50-60% confluency) were harvested, and 1×10⁵ cells per animal were implanted subcutaneously into female C57BL/6 mice (Taconic) in the right lower flank. Upon tumors reaching an average size of 100 mm³ in volume, animals were stratified into experimental, negative control or positive control groups. Experimental and negative control groups received oral administrations of test article (Butyricimonas faecihominis P40-F2a) and vehicle, respectively, once daily for the duration of the study. The positive control group received anti-PD-L1 antibody (200 via intraperitoneal route) every 4 days for the duration of the study. Tumor volume and body weights were assessed 3 times a week, and tumor weights were assessed at the end of the study.

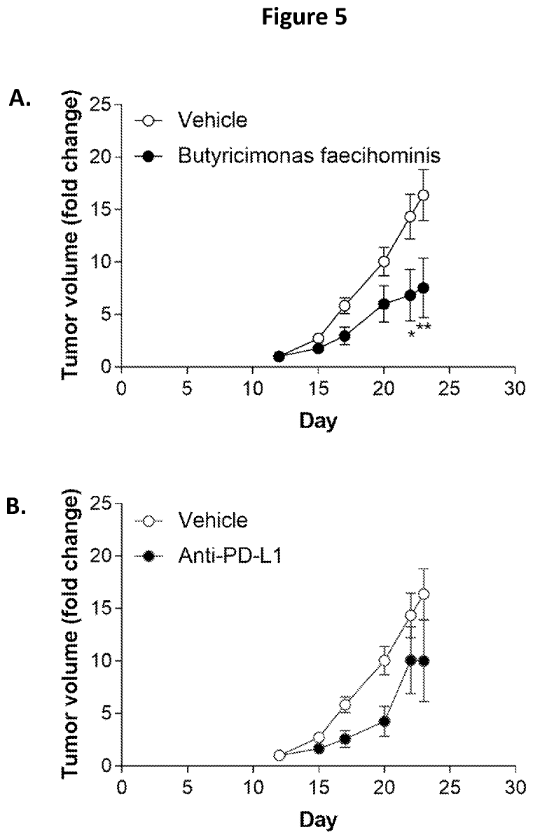

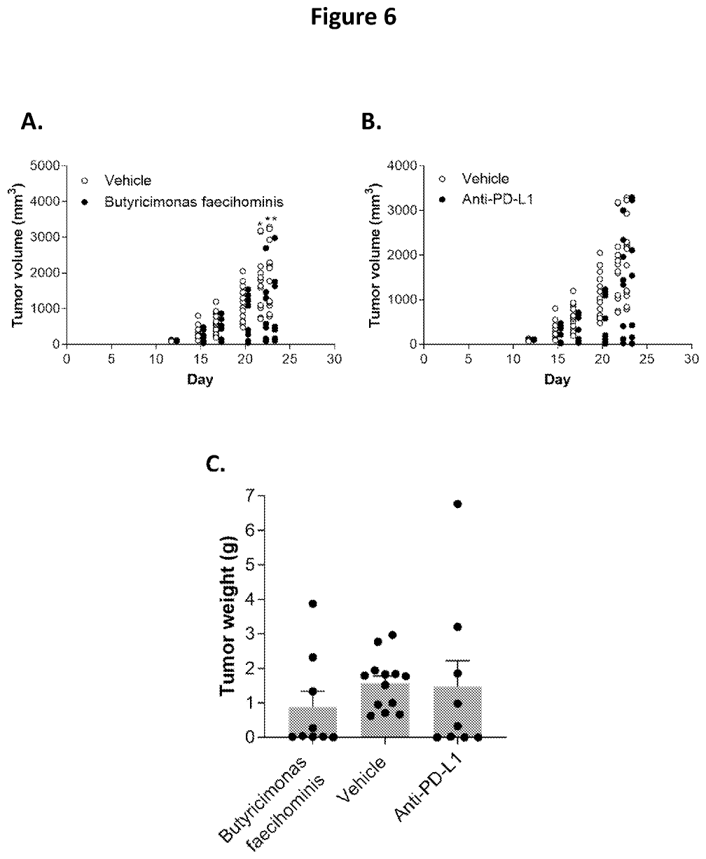

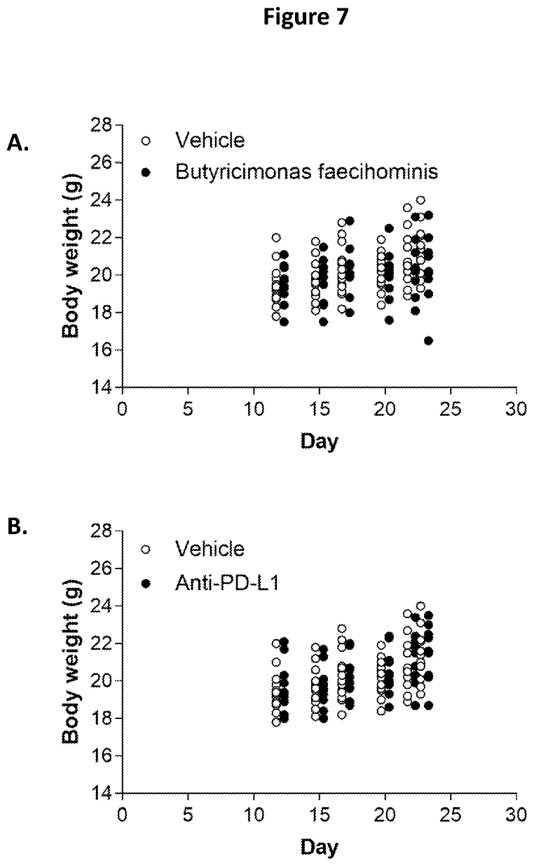

As shown in FIG. 5 and FIG. 6, administration of Butyricimonas faecihominis P40-F2a led to a significant reduction in tumor volume (displayed as fold-change in FIG. 5A and in mm³ in FIG. 6A), comparable to the reduction in tumor volume observed for animals receiving anti-PD-L1 antibody (displayed as fold-change in FIG. 5B and in mm³ in FIG. 6B). Tumor weights for animals administered Butyricimonas faecihominis P40-F2a were also reduced relative to both vehicle control and positive control groups (FIG. 6C). No changes in body weight were observed in animals receiving P40-F2a compared to those receiving vehicle and positive control antibody (FIGS. 7A and B).

5.2 CT26 Colon Carcinoma Model

5.2.1 Tumor model. CT-26 cells were thawed from low passage number stock, expanded using standard tissue culture technique, and maintained in DMEM with 10% fetal bovine plasma (FBS) in a 37° C. incubator with 5% CO₂. Cells growing in logarithmic phase (50-60% confluency) were harvested, and 1×10⁵ cells per animal were implanted subcutaneously into female BALB/c mice (Taconic) in the right lower flank. Upon tumors reaching an average size of 100 mm³ in volume, animals were stratified into experimental, negative control or positive control groups.

5.2.2 Butyricimonas faecihominis P40-F2a efficacy. In a first CT26 study, the positive control group received anti-PD-1 antibody (10 mg/kg, in 100 μl, via intraperitoneal route) every 3 days for the duration of the study. Experimental and negative control groups received oral administrations of test article (Butyricimonas faecihominis P40-F2a) and vehicle, respectively, once daily for the duration of the study. The positive control group received anti-PD-1 antibody (10 mg/kg, in 100 μl, via intraperitoneal route) every 3 days for the duration of the study. Tumor volume and body weights were assessed 3 times a week, and tumor weights were assessed at the end of the study.

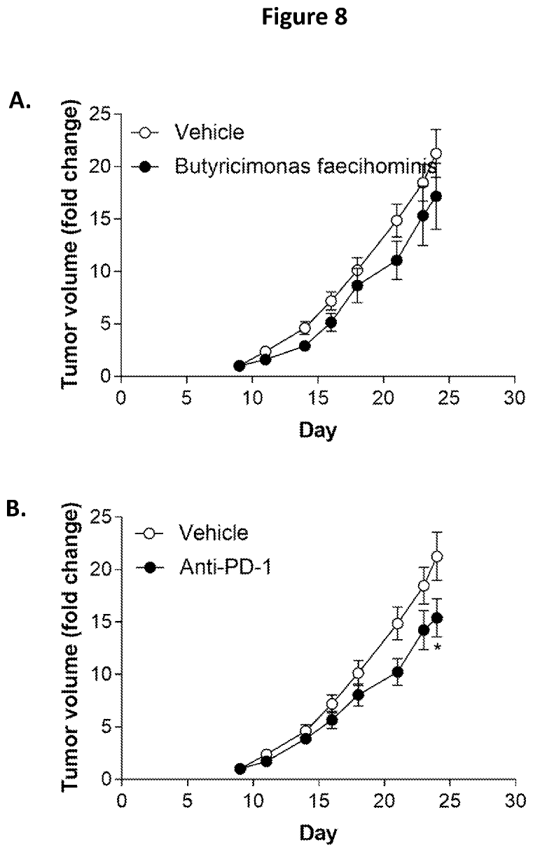

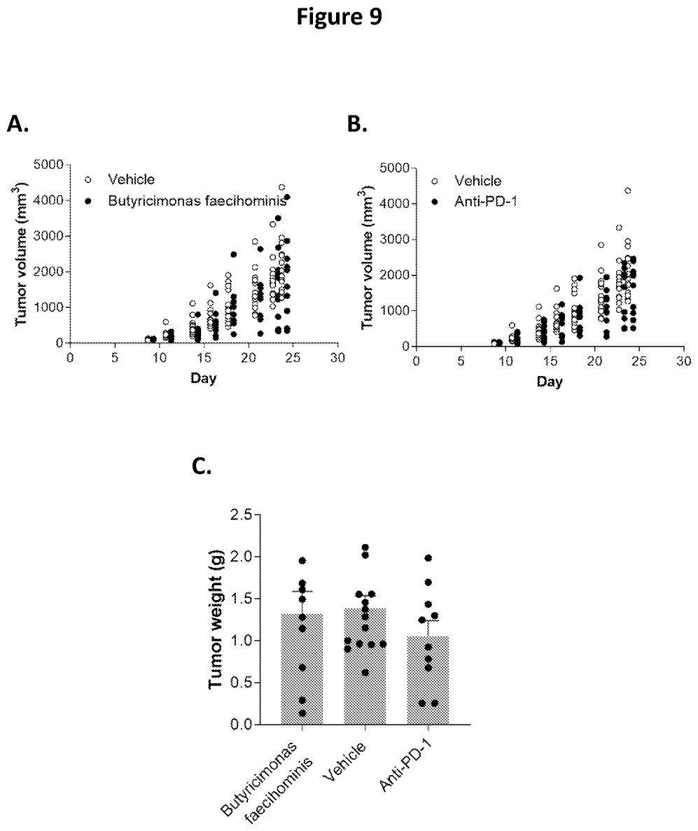

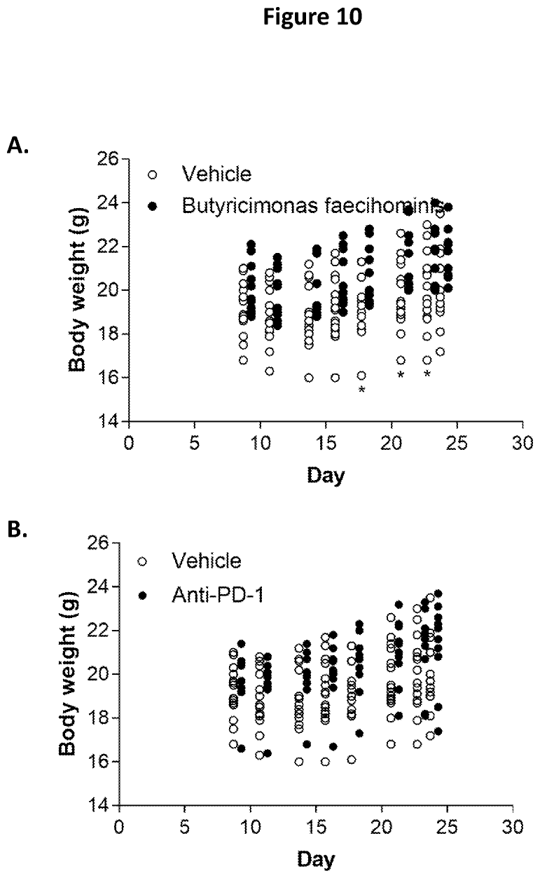

As shown in FIG. 8 and FIG. 9, administration of Butyricimonas faecihominis P40-F2a led to a reduction in tumor volume (displayed as fold-change in FIG. 8A and in mm³ in FIG. 9A), comparable to the reduction in tumor volume observed for animals receiving anti-PD-1 antibody (displayed as fold-change in FIG. 8B and in mm³ in FIG. 9B). Tumor weights for animals administered Butyricimonas faecihominis P40-F2a were also reduced relative to the vehicle control group (FIG. 9C). Inhibition of body weight reduction was observed in animals receiving P40-F2a compared to those receiving vehicle (FIG. 10A), which inhibition was improved relative to that observed in animals receiving anti-PD-1 antibody (FIG. 10B).

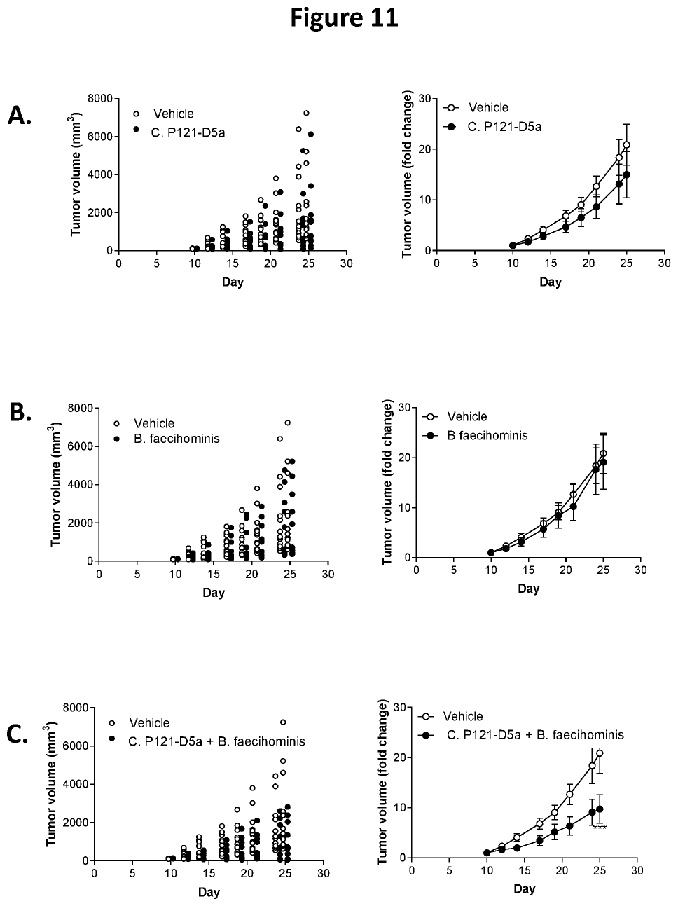

5.2.3 Butyricimonas faecihominis P40-F2a combined with Collinsella ASMB P121-D5a. In a second CT-26 study, tumor reduction was assessed in animals administered the combination of Butyricimonas faecihominis (P40-F2a) plus a Collinsella strain, Collinsella ASMB P121-D5a strain, in equal amounts (once daily). Single strain test groups were administered either Collinsella ASMB P121-D5a alone or Butyricimonas faecihominis P40-F2a alone, once daily for the duration of the study. The negative control group received vehicle only once daily for the duration of the study. As shown in FIG. 11A, administration of Collinsella ASMB P121-D5a alone resulted in a reduction of tumor growth compared to administration of vehicle only (displayed in mm³ in the left graph and as fold-change in the right graph). As shown in FIG. 11B, administration Butyricimonas faecihominis P40-F2a alone did not result in a significant reduction of tumor growth compared to administration of vehicle only (displayed in mm³ in the left graph and as fold-change in the right graph). However, as shown in FIG. 11C, administration of the combination of Collinsella ASMB P121-D5a plus Butyricimonas faecihominis P40-F2a resulted in significant tumor reduction which exceeded the tumor reduction observed for either strain alone (tumor volume versus vehicle only displayed in mm³ in the left graph and as fold-change in the right graph).

5.2.4 Butyricimonas faecihominis P40-F2a combined with Collinsella ASMB P121-D5a and anti-PD-1 antibody. In a third CT-26 study, the effects on tumor burden were assessed for animals receiving as test articles: Collinsella ASMB P121-D5a alone; Butyricimonas faecihominis P40-F2a alone; anti-PD-1 antibody alone; the combination of Collinsella ASMB P121-D5a plus Butyricimonas faecihominis P40-F2a; and the combination of Collinsella ASMB P121-D5a plus Butyricimonas faecihominis P40-F2a plus anti-PD-1 antibody. Animals in groups receiving bacterial test articles were administered once daily bacteria as single strains (mixed in equal volumes with vehicle), bacteria as 2-strain combinations (Collinsella ASMB P121-D5a strain and Butyricimonas faecihominis P40-F2a in equal amounts), or bacteria as 2-strain combinations (Collinsella ASMB P121-D5a strain and Butyricimonas faecihominis P40-F2a in equal amounts) plus anti-PD-1 antibody. Anti-PD-1 antibody was administered (alone as positive control or with a 2-strain combination) in an amount of 10 mg/kg, in 100 μl, via intraperitoneal route every 3 days for the duration of the study.

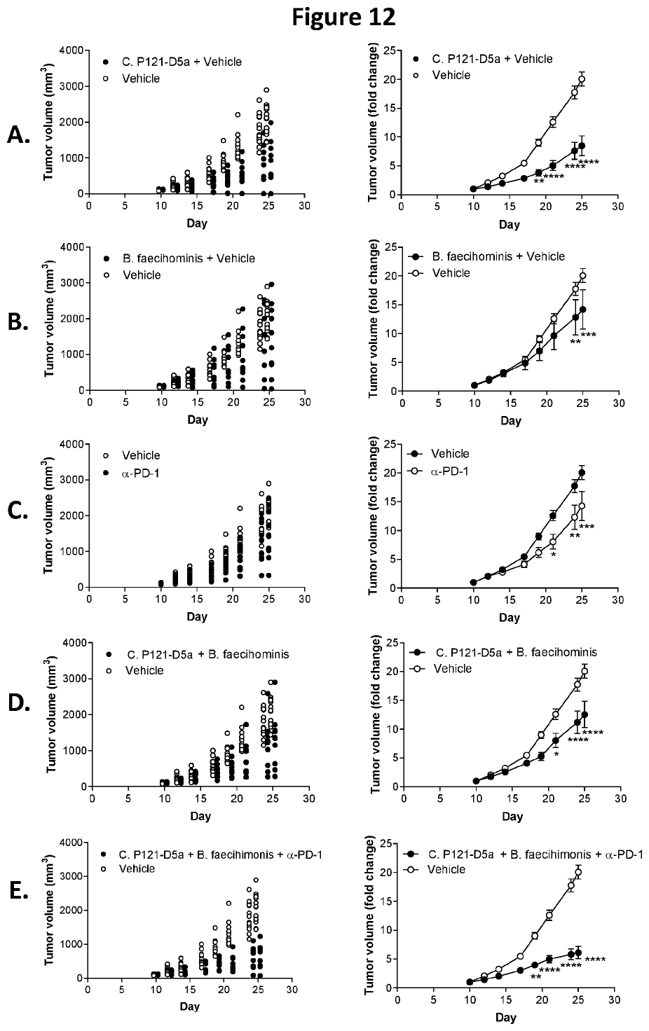

FIG. 12 provides tumor volume (mm³; left column) and tumor volume change (fold-change; right column) in animals treated with test articles (bacteria and/or antibody) versus animals treated with vehicle only. As shown, tumor reduction, when compared to administration of vehicle only, was observed in groups administered: Collinsella ASMB P121-D5a alone (FIG. 12A); Butyricimonas faecihominis P40-F2a alone (FIG. 12B); anti-PD-1 antibody alone (FIG. 12C); and the combination of Collinsella ASMB P121-D5a plus Butyricimonas faecihominis P40-F2a (FIG. 12D). However, the most significant tumor reduction was observed when animals received the combination of Collinsella ASMB P121-D5a plus Butyricimonas faecihominis P40-F2a plus anti-PD-1 antibody (FIG. 12E), which tumor reduction exceeded either of the single strain administrations, anti-PD-1 antibody only, and the two-strain combination without antibody.

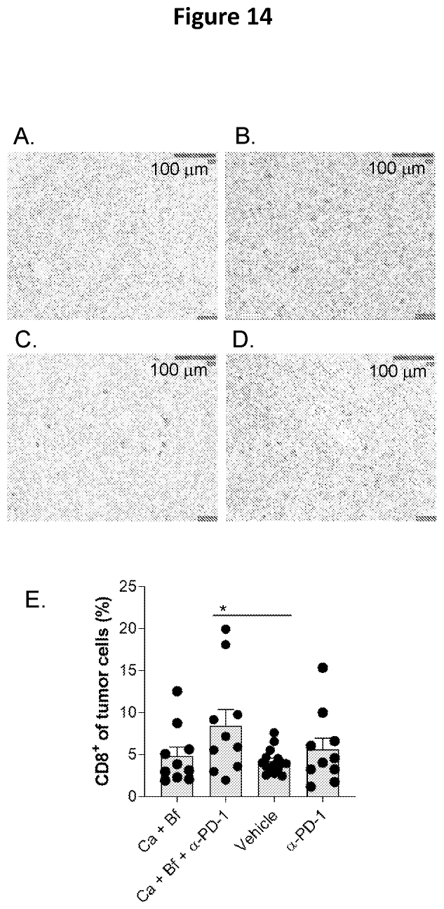

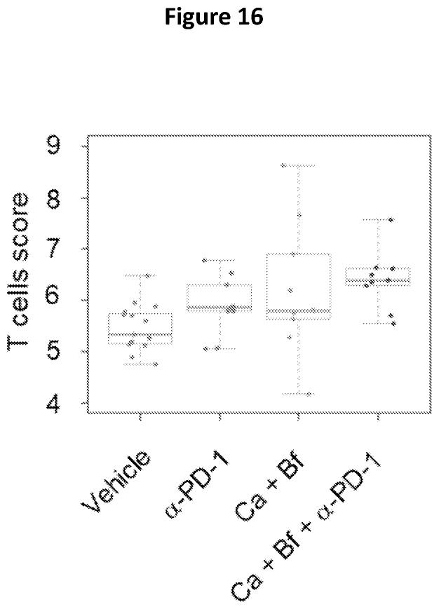

The combination of Collinsella ASMB P121-D5a and Butyricimonas faecihominis P40-F2a strains plus an anti-PD-1 antibody also increased tumor infiltration of T cells relative to administration with vehicle, the two-strain combination alone, and the anti-PD-1 antibody alone. FIG. 14 provides representative immunohistochemistry staining for CD8+ T cells in formalin-fixed paraffin-embedded (FFPE) tumor sections following treatment with (FIG. 14A) Collinsella ASMB P121-D5a+Butyricimonas faecihominis P40-F2a (Ca+Bf); (FIG. 14B) Collinsella ASMB P121-D5a+Butyricimonas faecihominis P40-F2a+α-PD-1 (Ca+Bf+α-PD-1); (FIG. 14C) Vehicle; or (FIG. 14D) α-PD-1. Tumor infiltration of T cells was also determined by Nanostring RNA-based cell type profiling. RNA was extracted and purified from tumor samples and analyzed using the Mouse PanCancer TO 360 Panel. T cell designation was based on expression of Cd3d, Cd3e, Cd3g, Cd6, Sh2d1a and Trat1. As shown in FIG. 16, each of anti-PD-1 antibody alone (α-PD-1), the combination of Collinsella ASMB P121-D5a and Butyricimonas faecihominis P40-F2a (Ca+Bf) strains, and the combination of Collinsella ASMB P121-D5a and Butyricimonas faecihominis P40-F2a strains and anti-PD-1 antibody (Ca+Bf+α-PD-1) increased tumor-infiltrating T cells relative to administration of vehicle only, with the highest T cell score observed for the combination of Ca+Bf+α-PD-1.

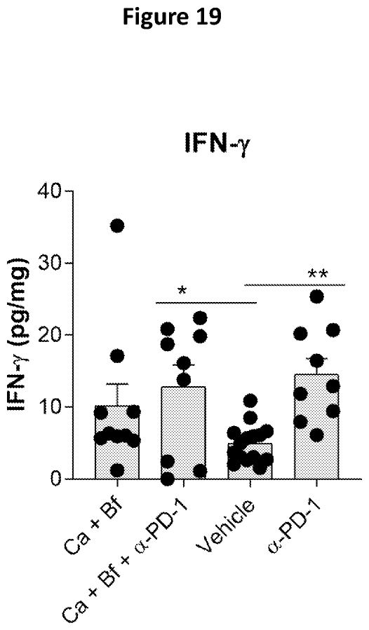

IFN-γ concentrations were also assessed in tumor samples by Meso Scale Discovery (MSD) analysis of tumor tissue homogenate and normalized to homogenate protein concentration. As shown in FIG. 19, each of anti-PD-1 antibody alone (α-PD-1), the combination of Collinsella ASMB P121-D5a and Butyricimonas faecihominis P40-F2a (Ca+Bf) strains, and the combination of Collinsella ASMB P121-D5a and Butyricimonas faecihominis P40-F2a strains and anti-PD-1 antibody (Ca+Bf+α-PD-1) increased IFN-γ concentrations in tumors relative to administration of vehicle alone.

5.2.5 Butyricimonas faecihominis P40-F2a combined with Alistipes senegalensis and anti-PD-1 antibody. Also in the third CT-26 study, the effects on tumor burden were assessed for animals receiving as test articles: Butyricimonas faecihominis (P40-F2a) alone; an isolated and purified strain of Alistipes, Alistipes senegalensis P150-D12a, alone; anti-PD-1 antibody alone; the combination of Alistipes senegalensis P150-D12a plus Butyricimonas faecihominis P40-F2a; and the combination of Alistipes senegalensis P150-D12a plus Butyricimonas faecihominis P40-F2a plus anti-PD-1 antibody. Animals in groups receiving bacterial test articles were administered once daily bacteria as single strains (mixed in equal volumes with vehicle), bacteria as 2-strain combinations (Alistipes senegalensis P150-D12a strain and Butyricimonas faecihominis P40-F2a in equal amounts), or bacteria as 2-strain combinations (Alistipes senegalensis P150-D12a strain and Butyricimonas faecihominis P40-F2a in equal amounts) plus anti-PD-1 antibody. Anti-PD-1 antibody was administered (alone as positive control or with a 2-strain combination) in an amount of 10 mg/kg, in 100 μl, via intraperitoneal route every 3 days for the duration of the study.

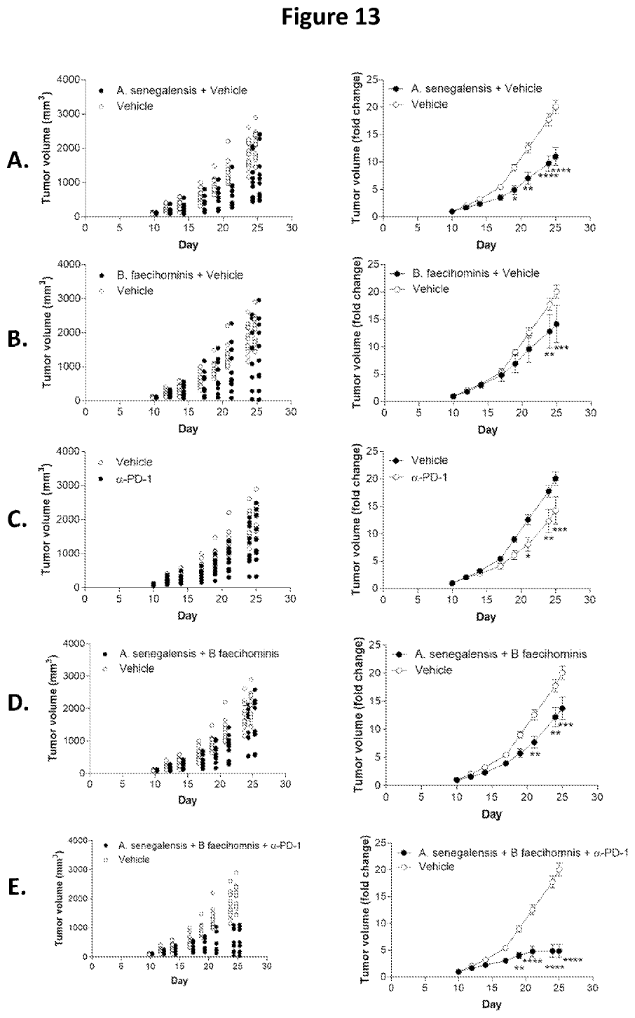

FIG. 13 provides tumor volume (mm³; left column) and tumor volume change (fold-change; right column) in animals treated with test articles (bacteria and/or antibody) versus animals treated with vehicle only. As shown, tumor reduction, when compared to administration of vehicle only, was observed in groups administered: Alistipes senegalensis P150-D12a alone (FIG. 13A); Butyricimonas faecihominis P40-F2a alone (FIG. 13B); anti-PD-1 antibody alone (FIG. 13C); and the combination of Alistipes senegalensis P150-D12a plus Butyricimonas faecihominis P40-F2a (FIG. 13D). However, the most significant tumor reduction was observed when animals received the combination of Alistipes senegalensis P150-D12a plus Butyricimonas faecihominis P40-F2a plus anti-PD-1 antibody (FIG. 13E), which tumor reduction exceeded either of the single strain administrations, anti-PD-1 antibody only, and the two-strain combination without antibody.

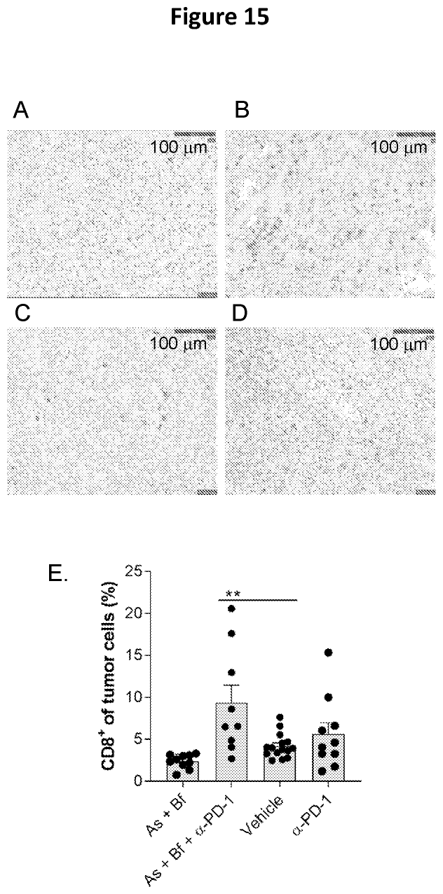

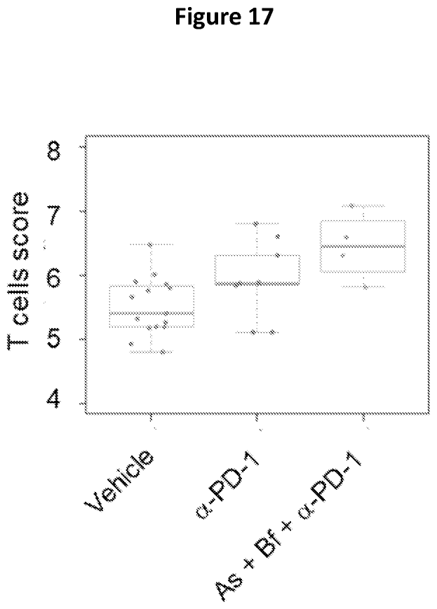

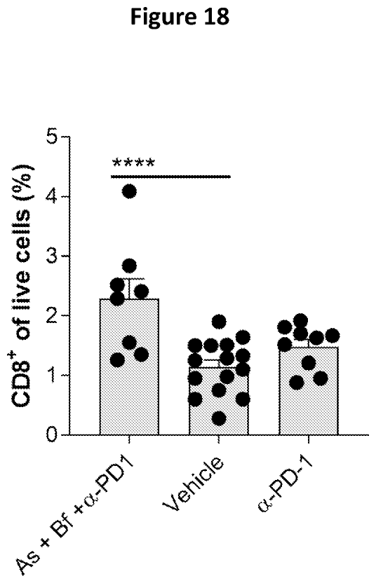

The combination of Alistipes senegalensis P150-D12a and Butyricimonas faecihominis P40-F2a strains plus an anti-PD-1 antibody also increased tumor infiltration of T cells relative to administration with vehicle, the two-strain combination alone, and the anti-PD-1 antibody alone. FIG. 15 provides representative immunohistochemistry staining for CD8+ T cells in formalin-fixed paraffin-embedded (FFPE) tumor sections following treatment with (FIG. 15A) Alistipes senegalensis P150-D12a+Butyricimonas faecihominis P40-F2a (As+Bf); (FIG. 15B) Alistipes senegalensis P150-D12a+Butyricimonas faecihominis P40-F2a+α-PD-1 (As+Bf+α-PD-1); (FIG. 15C) Vehicle; or (FIG. 15D) α-PD-1. Tumor infiltration of T cells was also determined by Nanostring RNA-based cell type profiling. RNA was extracted and purified from tumor samples and analyzed using the Mouse PanCancer TO 360 Panel. T cell designation was based on expression of Cd3d, Cd3e, Cd3g, Cd6, Sh2d1a and Trat1. As shown in FIG. 17, each of anti-PD-1 antibody alone (α-PD-1) and the combination of Alistipes senegalensis P150-D12a and Butyricimonas faecihominis P40-F2a strains and anti-PD-1 antibody (As+Bf+α-PD-1) increased tumor-infiltrating T cells relative to administration of vehicle only, with the highest T cell score observed for the combination of As+Bf+α-PD-1. T-cell infiltration was also determined by flow cytometry analysis of fresh tumor tissue. CD8⁺ T cells were identified by gating CD8⁺ CD4⁻ CD3⁺ CD45⁺ lymphocytes and quantified as percentage of live cells. As shown in FIG. 18, the combination of Alistipes senegalensis P150-D12a and Butyricimonas faecihominis P40-F2a strains plus an anti-PD-1 antibody (As+Bf+α-PD-1) increased tumor-infiltrating CD8⁺ T cells, relative to administration with vehicle or antibody alone.

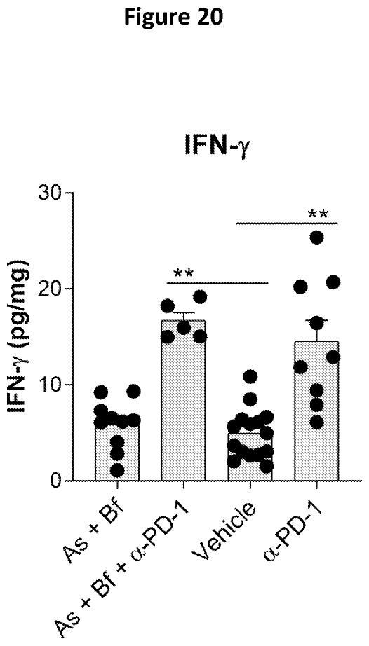

IFN-γ concentrations were also assessed in tumor samples by Meso Scale Discovery (MSD) analysis of tumor tissue homogenate and normalized to homogenate protein concentration. As shown in FIG. 20, each of anti-PD-1 antibody alone (α-PD-1), and the combination of Alistipes senegalensis P150-D12a and Butyricimonas faecihominis P40-F2a strains and anti-PD-1 antibody (As+Bf+α-PD-1) significantly increased IFN-γ concentrations in tumors relative to administration of vehicle only.

### Example 6—In Vivo Functional Activity of Bacterial Consortia Comprising Butyricimonas faecihominis and Lactobacillus ruminis

6.1 Efficacy of Lactobacillus ruminis in B16F10 Melanoma and CT26 Colon Carcinoma Models

Lactobacillus ruminis strain P167-B1a was tested for efficacy in two different well-validated mouse tumor models: (1) a subcutaneous B16F10 syngeneic melanoma model and (2) a subcutaneous CT26 syngeneic colon carcinoma model. Studies were conducted as described above (Section 5.1: B16F10 syngeneic melanoma model; Section 5.2.1: CT26 colon carcinoma model).

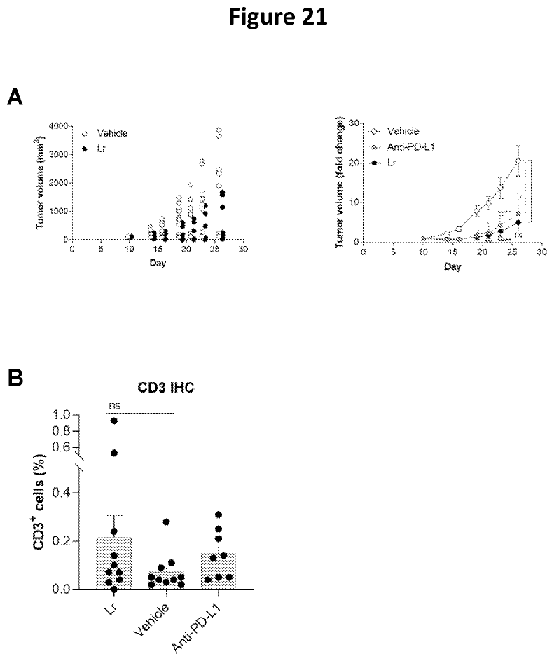

As shown in FIG. 21A, administration of Lactobacillus ruminis strain P167-B1a (Lr) led to a significant reduction in B16F10 tumor volume (displayed as fold-change in right panel and in mm³ in left panel), comparable to the reduction in tumor volume observed for animals receiving anti-PD-L1 antibody. As shown in FIG. 21B, immunohistochemistry (IHC) analysis of CD3⁺ cells in fixed tumor sections showed that administration of Lr led to a modest but non-significant increase in CD3⁺ cells, as a percent of non-necrotic cells.

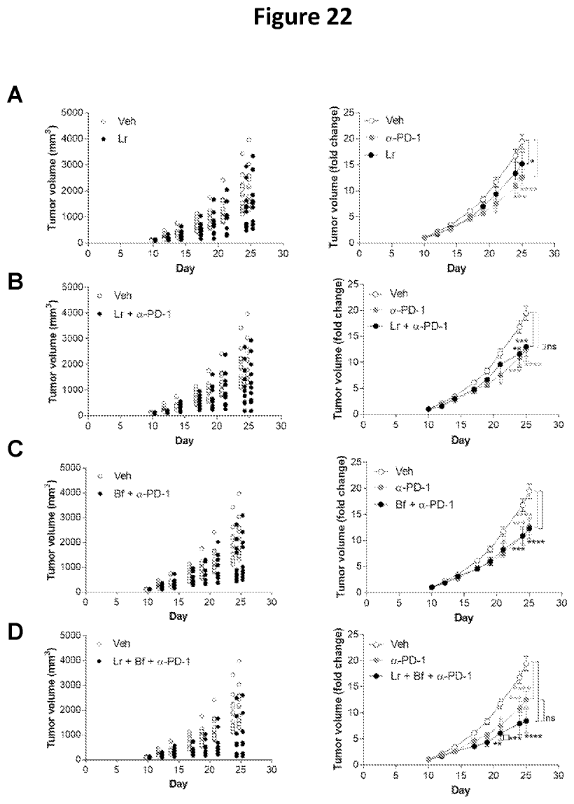

As shown in FIG. 22A, administration of Lr also led to a significant reduction in CT26 tumor volume (displayed as fold-change in right panel and in mm³ in left panel). As shown in FIG. 22B, administration of Lr in combination with an anti-PD-1 antibody did not significantly improve the anti-tumor effect of the Lr strain when administered alone, nor the anti-PD-1 antibody when administered alone.

6.2 Butyricimonas faecihominis combined with Lactobacillus ruminis and anti-PD-1 antibody. In an additional CT-26 study, the effects on tumor burden were assessed for animals receiving as test articles: Lactobacillus ruminis strain P167-B1a (Lr) alone; the combination of Lr plus anti-PD-1 antibody; the combination of Butyricimonas faecihominis P40-F2a (Bf) plus anti-PD-1 antibody; and the combination of Lr plus Bf plus anti-PD-1 antibody. Animals in groups receiving bacterial test articles were administered once daily bacteria as single strains (mixed in equal volumes with vehicle), bacteria as 2-strain combinations (Lr and Bf in equal amounts), or bacteria as 2-strain combinations (Lr and Bf in equal amounts) plus anti-PD-1 antibody. Anti-PD-1 antibody was administered (alone as positive control or with a 2-strain combination) in an amount of 10 mg/kg, in 100 μl, via intraperitoneal route every 3 days for the duration of the study.

FIG. 22 provides tumor volume (mm³; left column) and tumor volume change (fold-change; right column) in animals treated with test articles (bacteria and/or antibody) versus animals treated with vehicle only. Tumor reduction, when compared to administration of vehicle only, was observed in groups administered: Lr alone (FIG. 22A); Lr plus anti-PD-1 antibody (FIG. 22B); Bf plus anti-PD-1 antibody (FIG. 22C); and the combination of Lr plus Bf plus anti-PD-1 antibody (FIG. 22D). The most significant tumor reduction was observed when animals received the combination of Lr plus Bf plus anti-PD-1 antibody (FIG. 22D), which tumor reduction exceeded that of Lr alone, Lr plus antibody and Bf plus antibody. These results suggest that Lactobacillus ruminis and Butyricimonas faecihominis can synergize to potentiate the anti-tumor effect of checkpoint inhibitors such as anti-PD-1.

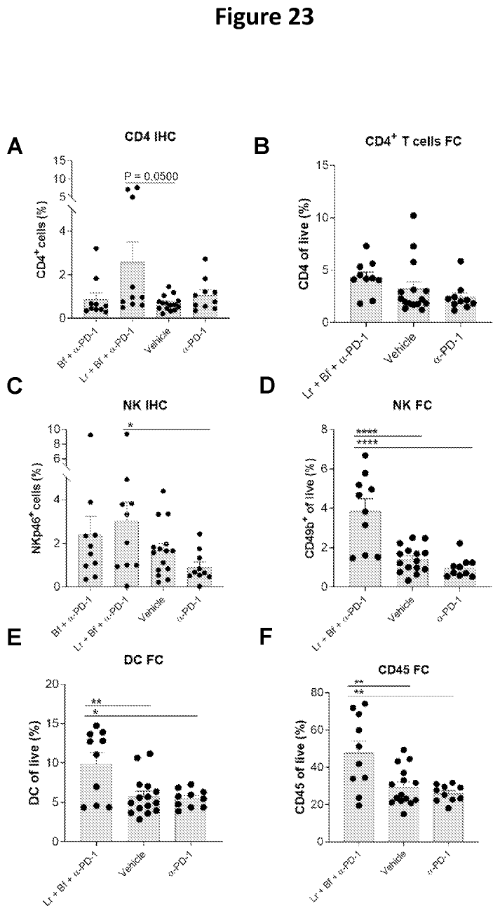

FIG. 23 depicts the effects of administration of a combination of Lactobacillus ruminis P167-B1a (Lr)+Butyricimonas faecihominis P40-F2a (Bf)+anti-PD-1 antibody on tumor infiltrating cells, compared to administration of anti-PD-1 antibody alone, as shown by immunohistochemistry (IHC) and flow cytometry (FC). FIG. 23A, B: CD4⁺ T cells; FIG. 23C, D: natural killer (NK) cells; FIG. 23E: dendritic cells (DC); and FIG. 23F: CD45⁺ leukocytes. Consistent with the potentiation of tumor reduction by the combination of Lr and Bf, this combination also appears to potentiate anti-PD-1 induced infiltration of immune cells into tumors.

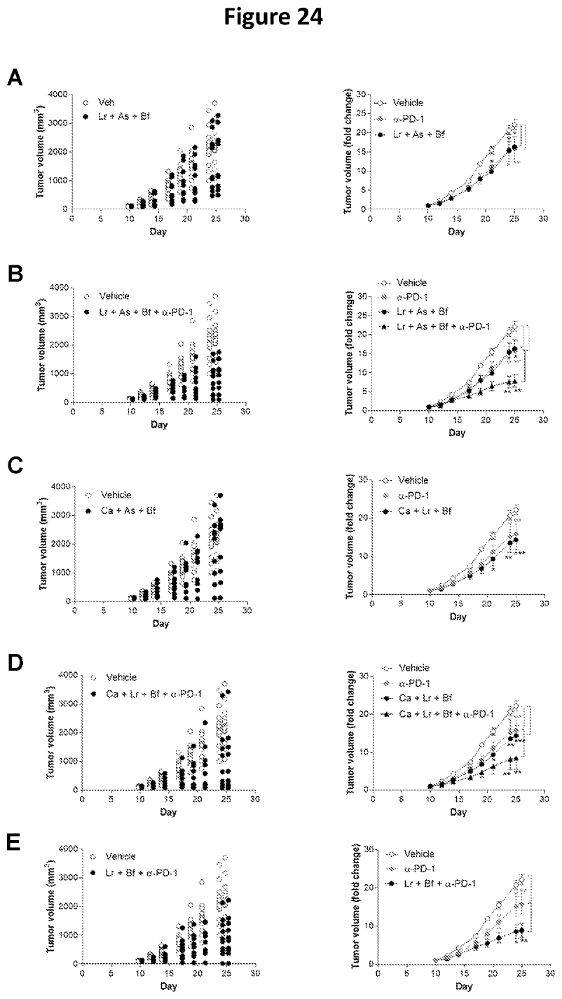

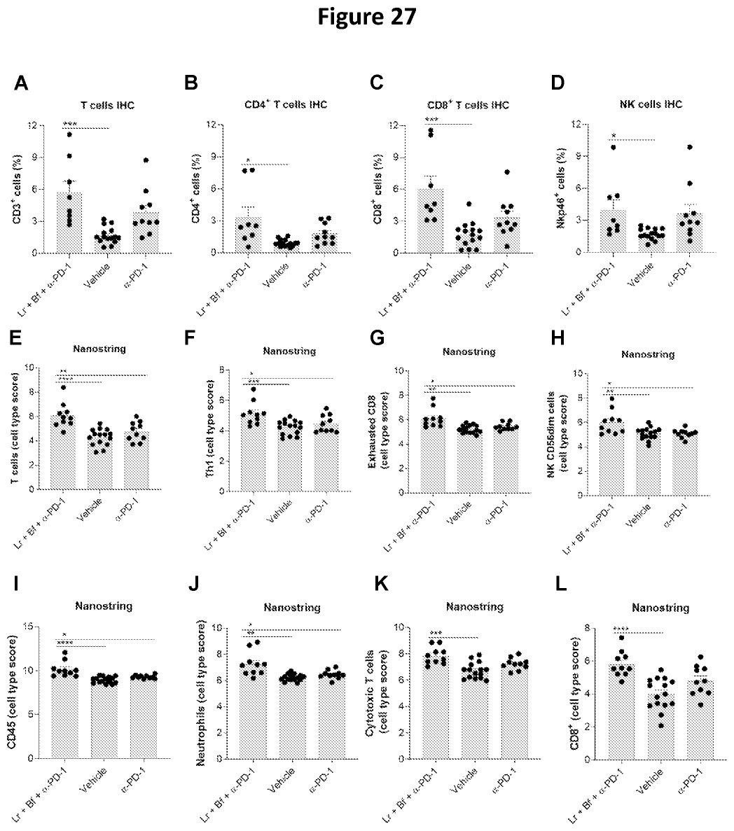

The results shown in FIG. 22D and FIG. 23 were corroborated and extended in an additional CT26 study. As shown in FIG. 24E, administration of Lr plus Bf plus anti-PD-1 antibody resulted in significantly enhanced tumor reduction compared to administration of anti-PD-1 alone. FIGS. 27A-D depict the corresponding effects of the Lr+Bf combination plus anti-PD-1 antibody on tumor-infiltrating immune cell populations, as a frequency of non-necrotic cells in fixed tumor sections by immunohistochemistry (IHC). Administration of the 2-strain combination plus anti-PD-1 antibody increased tumor infiltration of the following cell types: (FIG. 27A) CD3⁺ T cells; (FIG. 27B) CD4⁺ T cells; (FIG. 27C) CD8⁺ T cells; and (FIG. 27D) NK cells. Tumor-infiltrating immune cells were also scored by targeted gene expression using Nanostring platform from RNA extracted from frozen tumor pieces. As shown in FIGS. 27E-L, administration of the 2-strain combination plus anti-PD-1 antibody increased tumor infiltration relative to antibody alone for the following cell types: (E) T cells based on expression of: Cd3d, Cd3e, Cd3g, Cd6, Sh2d1a and Trat1; (F) Th1 cells based on expression of Tbx21; (G) exhausted CD8 T cells based on expression of: Cd244, Eomes, Lag3, and Ptger4; (H) NK CD56dim cells based on expression of: Il21r, Kir3d11, and Kir3d12; (I) CD45⁺ cells based on expression of Ptprc; (J) neutrophils based on expression of: Ceacam3, Csf3r, Fcgr4 and Fpr1; (K) cytotoxic T cells based on expression of: Ctsw, Gzma, Gzmb, Klrb1, Klrd1, Klrk1, Nkg7, and Prf1; and (L) CD8⁺ cells based on expression of Cd8a and Cd8b1.

6.3 Butyricimonas faecihominis Combined with Lactobacillus ruminis, Alistipes Senegalensis and Anti-PD-1 Antibody.

In an additional CT-26 study, the combination of Butyricimonas faecihominis P40-F2a (Bf), Lactobacillus ruminis strain P167-B1a (Lr) and Alistipes senegalensis P150-D12a (As) was administered to tumor-bearing mice, with and without co-administration of anti-PD-1 antibody. Additional test articles included Vehicle only and anti-PD-1 antibody alone. Animals in groups receiving bacterial test articles were administered once daily the 3-strain combination (Lr, Bf and As in equal amounts), or 3-strain combination plus anti-PD-1 antibody. Anti-PD-1 antibody was administered (alone as positive control or with the 3-strain combination) in an amount of 10 mg/kg, in 100 μl, via intraperitoneal route every 3 days for the duration of the study.

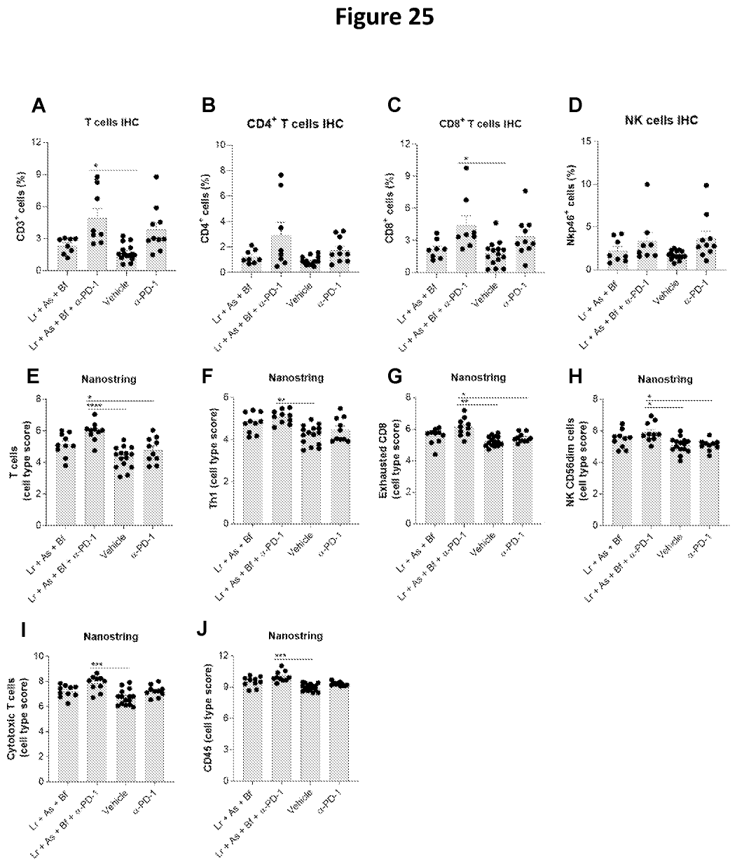

As shown in FIG. 24A, the combination of Lr+As+Bf induced a reduction in tumor volume comparable to anti-PD-1 antibody alone. Co-administration of anti-PD-1 antibody with the 3-strain combination greatly potentiated the anti-tumor effect relative to the anti-PD-1 alone and this 3-strain combination without antibody (FIG. 24B), suggesting a synergistic effect between the 3-strain combination and checkpoint inhibitor. FIGS. 25A-D depict the corresponding effects of the Lr+As+Bf combination plus anti-PD-1 antibody on tumor-infiltrating immune cell populations, as a frequency of non-necrotic cells in fixed tumor sections by immunohistochemistry (IHC). Administration of the 3-strain combination plus anti-PD-1 antibody increased tumor infiltration of the following cell types: (FIG. 25A) CD3⁺ T cells; (FIG. 25B) CD4⁺ T cells; and (FIG. 25C) CD8⁺ T cells. Increases were relative to administration of anti-PD-1 antibody only and the 3-strain combination without antibody, which supports the synergy between the 3-strain combination and checkpoint inhibitor.

Tumor-infiltrating immune cells were also scored by targeted gene expression using Nanostring platform from RNA extracted from frozen tumor pieces. As shown in FIGS. 25E-J, administration of the 3-strain combination plus anti-PD-1 antibody increased tumor infiltration relative to antibody alone for the following cell types: (E) T cells based on expression of the following genes: Cd3d, Cd3e, Cd3g, Cd6, Sh2d1a and Trat1; (F) Th1 cells based on expression of Tbx21; (G) Exhausted CD8 T cells based on expression of the following genes: Cd244, Eomes, Lag3 and Ptger4; (H) NK CD56dim cells based on expression of: Il21r, Kir3d11 and Kir3d12; (I) Cytotoxic T cells based on expression of: Ctsw, Gzma, Gzmb, Klrb1, Klrd1, Klrk1, Nkg7 and Prf1; and (J) CD45⁺ cells based on expression of Ptprc.

6.4 Butyricimonas faecihominis Combined with Lactobacillus ruminis, Collinsella ASMB and Anti-PD-1 Antibody.

The combination of Butyricimonas faecihominis P40-F2a (Bf), Lactobacillus ruminis strain P167-B1a (Lr) and Collinsella ASMB P121-D5a (Ca) was administered to CT26 tumor-bearing mice, with and without co-administration of anti-PD-1 antibody. Additional test articles included Vehicle only and anti-PD-1 antibody alone. Animals in groups receiving bacterial test articles were administered once daily the 3-strain combination (Lr, Bf and Ca in equal amounts), or 3-strain combination plus anti-PD-1 antibody. Anti-PD-1 antibody was administered (alone as positive control or with the 3-strain combination) in an amount of 10 mg/kg, in 100 μl, via intraperitoneal route every 3 days for the duration of the study.

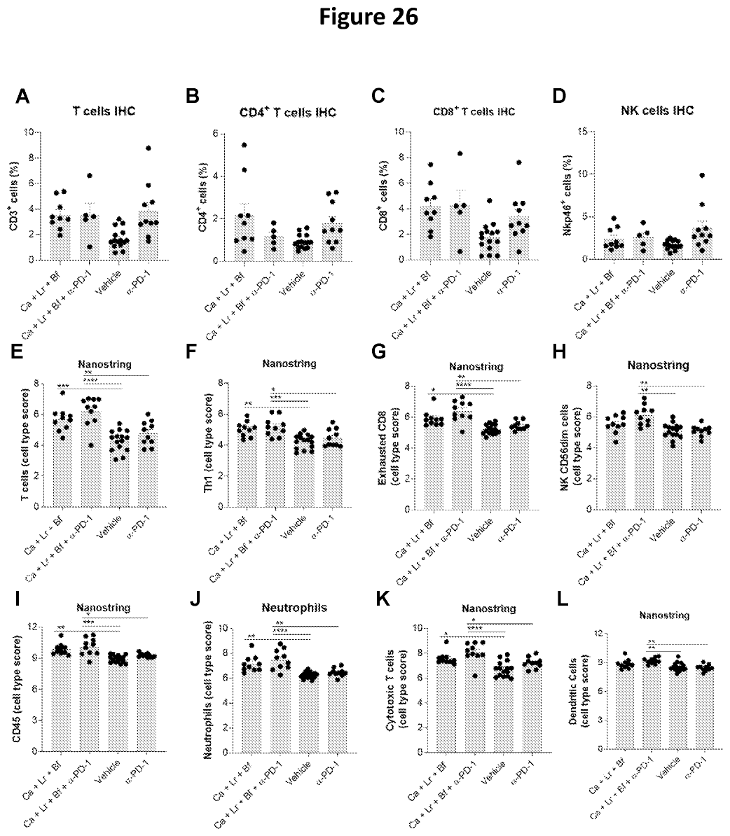

As shown in FIG. 24C, the combination of Ca+Lr+Bf induced a reduction in tumor volume comparable to anti-PD-1 antibody alone. Co-administration of Anti-PD-1 antibody with the 3-strain combination greatly potentiated the anti-tumor effect relative to the anti-PD-1 alone and the 3-strain combination without antibody (FIG. 24D), suggesting a synergistic effect between this 3-strain combination and checkpoint inhibitor. FIGS. 26A-D depict the effects of the Ca+Lr+Bf combination plus anti-PD-1 antibody on tumor-infiltrating immune cell populations, as a frequency of non-necrotic cells in fixed tumor sections by immunohistochemistry (IHC). A trend of increased CD8+ T cell infiltration was observed with administration of the 3-strain combination with and without anti-PD-1 antibody, relative to anti-PD-1 antibody alone (FIG. 26C). Tumor-infiltrating immune cells were also scored by targeted gene expression using Nanostring platform from RNA extracted from frozen tumor pieces. As shown in FIGS. 26E-J, administration of the 3-strain combination plus anti-PD-1 antibody increased tumor infiltration relative to antibody alone for the following cell types: (E) T cells based on expression of the following genes: Cd3d, Cd3e, Cd3g, Cd6, Sh2d1a and Trat1; (F) Th1 cells based on expression of Tbx21; (G) exhausted CD8 T cells based on expression of: Cd244, Eomes, Lag3 and Ptger4; (H) NK CD56dim cells based on expression of: Il21r, Kir3d11, and Kir3d12; (I) CD45⁺ cells score based on expression of Ptprc; (J) neutrophils based on expression of: Ceacam3, Csf3r, Fcgr4 and Fpr1; (K) cytotoxic T cells based on expression of: Ctsw, Gzma, Gzmb, Klrb1, Klrd1, Klrk1, Nkg7 and Prf1; and (L) dendritic cells based on expression of: Ccl2, Cd209e and Hsd11b1.

6.5 Lyophilized Formulations of Butyricimonas faecihominis and Lactobacillus ruminis.

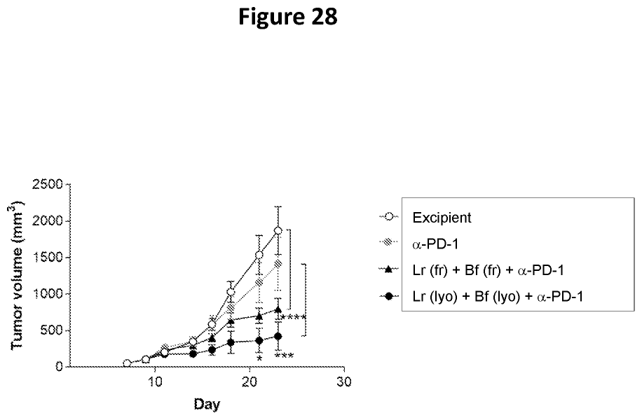

Butyricimonas faecihominis P40-F2a (Bf) and Lactobacillus ruminis P167-B1a (Lr) strains were lyophilized and assessed for anti-tumor efficacy in combination with anti-PD-1 antibody in the CT26 tumor model. As shown in FIG. 28, co-administration of anti-PD-1 antibody plus a combination of Lr and Bf strains reconstituted from lyophilized bacterial stocks (Lr (lyo)+Bf (lyo)+α-PD-1) retained strong anti-tumor efficacy. For reference, strong anti-tumor efficacy was also observed with co-administration of anti-PD-1 antibody plus a combination of Lr and Bf strains reconstituted from frozen bacterial stocks ((Lr (fr)+Bf (fr)+α-PD-1).

### Example 7—In Vivo Functional Activity of Bacterial Consortia Comprising Butyricimonas faecihominis and Other Strains

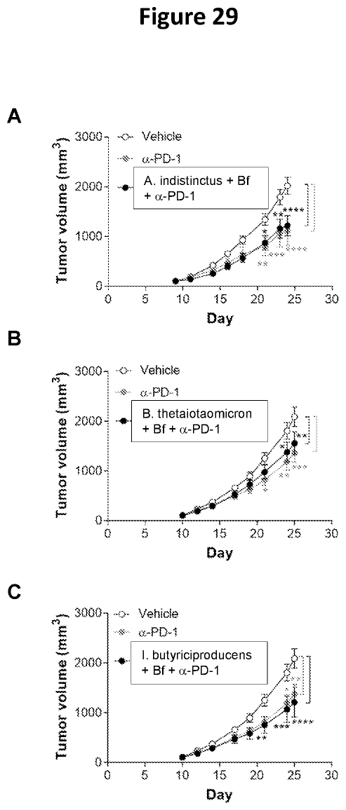

Combinations of Butyricimonas faecihominis P40-F2a (Bf) and other bacterial strains (Alistipes indistinctus, Bacteroides thetaiotaomicron and Intestinimonas butyriciproducens,) were assessed in the CT26 tumor model for their ability to potentiate the anti-tumor activity of checkpoint inhibitor antibody. Animals in groups receiving bacterial test articles were administered once daily bacteria as 2-strain combinations (Bf and the second strain in equal amounts) plus anti-PD-1 antibody. Anti-PD-1 antibody was administered (alone as positive control or with a 2-strain combination) in an amount of 10 mg/kg, in 100 μl, via intraperitoneal route every 3 days for the duration of the study. FIG. 29 depicts administration of an anti-PD-1 antibody alone compared to: (A) a combination of Alistipes indistinctus and Butyricimonas faecihominis P40-F2a (Bf) strains and an anti-PD-1 antibody; (B) a combination of Bacteroides thetaiotaomicron and Bf strains and an anti-PD-1 antibody; and (C) a combination of Intestinimonas butyriciproducens and Bf strains and an anti-PD-1 antibody. None of the 2-strain combinations plus anti-PD-1 antibody significantly increased tumor reduction compared to anti-PD-1 alone.

### INCORPORATION BY REFERENCE

The entire disclosure of each of the patent and scientific documents referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes that come within the meaning and range of equivalency of the claims are intended to be embraced therein.

What is claimed is:

1. A composition comprising:
a bacterial strain of the genus Butyricimonas that comprises a 16s rRNA gene sequence with at least about 98% sequence identity to the polynucleotide sequence of SEQ ID NO: 846; and
an excipient, diluent and/or carrier;
wherein the bacterial strain is lyophilized, freeze dried or spray dried.

2. The composition of claim 1, wherein the bacterial strain is capable of increasing production of one or more pro-inflammatory cytokines by a human macrophage, monocyte, peripheral blood mononuclear cell or monocyte-derived dendritic cell in vitro.

3. The composition of claim 1, wherein the pro-inflammatory cytokine is selected from the group consisting of interferon gamma (IFN-γ), interleukin 1 beta (IL-1β), interleukin 6 (IL-6), interleukin 12 (IL-12, e.g., IL-12p40, IL-12p70), interleukin 23 (IL-23), interleukin 27 (IL-27), tumor necrosis factor (TNF), and/or TNF-related apoptosis inducing ligand (TRAIL).

4. The composition of any one of claims 1 to 3, wherein the composition comprising a bacterial strain of the genus Butyricimonas is capable of increasing infiltration of T cells into a tumor.

5. The composition of any one of claims 1 to 4, wherein the bacterial strain comprises a 16s rRNA gene sequence with at least about 98.5%, 99% or 99.5% sequence identity to the polynucleotide sequence of SEQ ID NO: 846.

6. The composition of any one of claims 1 to 5, wherein the bacterial strain shares at least 70% DNA-DNA hybridization with strain Butyricimonas faecihominis P40-F2a, having the deposit accession number DSM 33411.

7. The composition of any one of claims 1 to 6, wherein the bacterial strain comprises a nucleotide sequence having at least about 70% identity to any one of SEQ ID NOs: 1-845.

8. The composition of any one of claims 1 to 7, wherein the bacterial strain comprises a genome having at least 95% average nucleotide identity (ANI) with the genome of Butyricimonas faecihominis strain P40-F2a, having the deposit accession number DSM 33411.

9. The composition of any one of claims 1 to 8, wherein the bacterial strain comprises a genome having at least 96.5% average nucleotide identity (ANI) and at least 60% alignment fraction (AF) with the genome of Butyricimonas faecihominis strain P40-F2a, having the deposit accession number DSM 33411.

10. The composition of any one of claims 1 to 9, wherein the bacterial strain is Butyricimonas faecihominis P40-F2a, having the deposit accession number DSM 33411.

11. The composition of any one of claims 1 to 10, wherein the composition is formulated as an enteric formulation.

12. The composition of claim 11, wherein the enteric formulation is formulated as a capsule, tablet, caplet, pill, troche, lozenge, powder, or granule.

13. The composition of any one of claims 1 to 12, wherein the composition is formulated as a suppository, suspension, emulsion, or gel.

14. The composition of any one of claims 1 to 13, wherein the composition comprises at least 1×10³ CFU of the bacterial strain.

15. The composition of any one of claims 1 to 14, wherein the composition comprises a therapeutically effective amount of the bacterial strain sufficient to prevent or treat a disorder when administered to a subject in need thereof.

16. The composition of claim 15, wherein the disorder is cancer.

17. The composition of claim 16, wherein the cancer comprises a solid tumor, soft tissue tumor, hematopoietic tumor or metastatic lesion.

18. The composition of claim 16, wherein the cancer is selected from the group consisting of leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, and Richter's Syndrome (Richter's Transformation).

19. The composition of claim 16, wherein the cancer is selected from the group consisting of a sarcoma, adenocarcinoma, and carcinoma.

20. The composition of claim 16, wherein the cancer is selected from the group consisting of head and neck cancer (including pharynx), thyroid cancer, lung cancer (small cell or non-small cell lung carcinoma (NSCLC)), breast cancer, lymphoid cancer, gastrointestinal cancer (e.g., oral, esophageal, stomach, liver, pancreas, small intestine, colon and rectum, anal canal), genital cancer, genitourinary tract cancer (e.g., renal, urothelial, bladder, ovarian, uterine, cervical, endometrial, prostate, testicular), CNS cancer (e.g., neural or glial cells, e.g., neuroblastoma or glioma), skin cancer (e.g., melanoma) and colorectal cancer (CRC).

21. The composition of any one of claims 1 to 20, wherein the excipient is selected from the group consisting of a filler, a binder, a disintegrant, and any combination(s) thereof.

22. The composition of any one of claims 1 to 20, wherein the excipient is selected from the group consisting of cellulose, polyvinyl pyrrolidone, silicon dioxide, stearyl fumarate or a pharmaceutically acceptable salt thereof, and any combination(s) thereof.

23. The composition of any one of claims 1 to 22, wherein the composition further comprises a cryoprotectant.

24. The composition of claim 23, wherein the cryoprotectant is selected from the group consisting of a fructoligosaccharide, trehalose, and a combination thereof.

25. The composition of claim 24, wherein the fructoligosaccharide is Raftilose®.

26. The composition of any one of claims 1 to 25, wherein the composition is suitable for bolus administration or bolus release.

27. The composition of any one of claims 1 to 26, wherein the bacterial strain is capable of at least partially colonizing an intestine of a human subject.

28. The composition of any one of claims 1 to 27, wherein the composition is suitable for oral delivery to a subject.

29. The composition of any one of claims 1 to 28, wherein the bacterial strain is viable.

30. The composition of any one of claims 1 to 29, wherein the composition comprises at least one more additional bacterial strain(s).

31. The composition of any one of claims 1 to 30, wherein upon storage for 6 months at 4° C., the composition loses at most 3 log colony forming units (cfus) of the bacterial strain.

32. The composition of any one of claims 1 to 31, wherein the composition further comprises one more additional bacterial strains.

33. The composition of claim 32, wherein the one or more additional bacterial strains is selected from the group consisting of a strain from the genus Collinsella, a strain from the genus Alistipes, and a strain from the genus Lactobacillus, and any combinations thereof.

34. The composition of claim 33, wherein the strain from the genus Collinsella is a strain of Collinsella ASMB.

35. The composition of claim 34, wherein the strain of Collinsella ASMB is Collinsella ASMB P121-D5a, deposited under accession number DSM 33276.

36. The composition of any one of claims 32-35, wherein the strain from the genus Alistipes is a strain of Alistipes senegalensis.

37. The composition of claim 36, wherein the strain of Alistipes senegalensis is Alistipes senegalensis strain P150-D12a, deposited under accession number DSM 33382.

38. The composition of any one of claims 32-37, wherein the strain from the genus Lactobacillus is a strain of Lactobacillus ruminis.

39. The composition of claim 38, wherein the strain of Lactobacillus ruminis is Lactobacillus ruminis strain P167-B1a, deposited under accession number DSM 33536.

40. A food product comprising the composition of any one of claims 1 to 39.

41. A method of treating cancer in a subject in need thereof, the method comprising administering a therapeutically effective amount of the composition of any one of claims 1-39 to the subject.

42. The method of claim 41, wherein the cancer comprises a solid tumor, soft tissue tumor, hematopoietic tumor or metastatic lesion.

43. The method of claim 41, wherein the cancer is selected from the group consisting of leukemia, acute leukemia, acute lymphoblastic leukemia (ALL), B-cell, T-cell or FAB ALL, acute myeloid leukemia (AML), chronic myelocytic leukemia (CIVIL), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, and Richter's Syndrome (Richter's Transformation).

44. The method of claim 41, wherein the cancer is selected from the group consisting of a sarcoma, adenocarcinoma, and carcinoma.

45. The method of claim 41, wherein the cancer is selected from the group consisting of head and neck cancer (including pharynx), thyroid cancer, lung cancer (small cell or non-small cell lung carcinoma (NSCLC)), breast cancer, lymphoid cancer, gastrointestinal cancer (e.g., oral, esophageal, stomach, liver, pancreas, small intestine, colon and rectum, anal canal), genital cancer, genitourinary tract cancer (e.g., renal, urothelial, bladder, ovarian, uterine, cervical, endometrial, prostate, testicular), CNS cancer (e.g., neural or glial cells, e.g., neuroblastoma or glioma), skin cancer (e.g., melanoma) and colorectal cancer (CRC).

46. A method of modifying a gut microbiome in a subject, the method comprising administering a therapeutically effective amount of the composition of any one of claims 1 to 39 to the subject.

47. A method of treating a dysbiosis in a subject in need thereof, the method comprising administering a therapeutically effective amount of the composition of any one of claims 1 to 39 to the subject.

48. The method of any one of claims 41-47, further comprising administering a prebiotic to the subject.

49. The method of any one of claims 41-48, wherein the subject is selected from the group consisting of a human, a companion animal, or a livestock animal.

50. The method of any one of claims 41-49, wherein the method further comprises administering an immune checkpoint inhibitor to the subject.

51. The method of claim 50, wherein the immune checkpoint inhibitor is selected from the group consisting of PD-1 antagonist, PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist and TIGIT antagonist.

52. The method of claim 50, wherein the immune checkpoint inhibitor is a PD-1 or PD-L1 inhibitor.

53. The method of claim 52, wherein the PD-1 inhibitor is an anti-PD-1 antibody.

54. The method of claim 52, wherein the PD-L1 inhibitor is an anti-PD-L1 antibody.
